(19) United States
(12) Patent Application Publication
(10) Pub. No.: US 2020/0003769 A1
(43) Pub. Date: Jan. 2, 2020

(54) COMPOSITIONS, DEVICES, AND METHODS OF ATTENTION DEFICIT DISORDER/ATTENTION DEFICIT HYPERACTIVITY DISORDER (ADD/ADHD) SENSITIVITY TESTING

(71) Applicant: Biomerica, Inc., Irvine, CA (US)

(72) Inventors: Zackary Irani-Cohen, Irvine, CA (US); Elisabeth Laderman, Irvine, CA (US)

(73) Assignee: Biomerica, Inc., Irvine, CA (US)

(21) Appl. No.: 16/441,902

(22) Filed: Jun. 14, 2019

Related U.S. Application Data
(63) Continuation of application No. PCT/IB2017/058023, filed on Dec. 15, 2017.
(60) Provisional application No. 62/434,957, filed on Dec. 15, 2016.

(51) Int. Cl. G01N 33/543

(52) U.S. Cl. CPC G01N 33/54306; G01N 33/02; G01N 2800/305

(57) ABSTRACT

Contemplated test kits, diagnostic apparatus and methods using same for food sensitivity are based on rational-based selection of food preparations with established discriminatory p-value. Kits and diagnostic apparatus include those with a minimum number of food preparations that have an average discriminatory p-value of ≤0.07 as determined by their raw p-value or an average discriminatory p-value of ≤0.10 as determined by FDR multiplicity adjusted p-value. In further contemplated aspects, compositions and methods for food sensitivity are also stratified by gender to further enhance predictive value.

### RELATED APPLICATIONS

This application is a Continuation of International Application No. PCT/IB2017/058023, filed Dec. 15, 2017, which claims priority to U.S. Provisional Patent Application No. 62/434,957, filed Dec. 15, 2016, and entitled “Compositions, Devices, And Methods of Attention Deficit Disorder/Attention Deficit Hyperactivity Disorder (ADD/ADHD) Sensitivity Testing.” Each of the foregoing applications is incorporated herein by reference in its entirety.

### FIELD

The field of the disclosure is related to the identification of food items that trigger and/or exacerbate ADD/ADHD, particularly as it relates to the testing, identification and possible elimination of selected food items as trigger foods for patients diagnosed with or suspected to have ADD/ADHD.

### BACKGROUND

The background description includes information that may be useful in understanding the present disclosure. It is not an admission that any of the information provided herein is prior art or relevant to the presently claimed invention, or that any publication specifically or implicitly referenced is prior art.

Food sensitivity, especially as it relates to ADD/ADHD (a type of neurodevelopmental psychiatric disorder), often presents with significant problems of attention and/or hyperactivity, and underlying causes of ADD/ADHD are not well understood in the medical community. Most typically, ADD/ADHD is diagnosed by an assessment of a person's childhood behavioral and mental development. Unfortunately, treatment of ADD/ADHD is often less than effective and may present new difficulties due to neurochemical modulatory effects. Elimination of one or more food items has also shown promise in at least reducing incidence and/or severity of the symptoms. However, ADD/ADHD is often quite diverse with respect to dietary items triggering symptoms, and no standardized test to help identify trigger food items with a reasonable degree of certainty is known, leaving such patients often to trial-and-error.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Accordingly, there is still a need for compositions, devices, and methods of food sensitivity testing, especially for identification and possible elimination of trigger foods for patients identified with or suspected of having ADD/ADHD.

### SUMMARY

The subject matter described herein provides systems and methods for identifying of food items that trigger and/or exacerbate ADD/ADHD symptomology in patients diagnosed with or suspected to have ADD/ADHD, or which alleviate ADD/ADHD symptomology when removed. One aspect of the disclosure is a test kit with for identifying food items that trigger and/or exacerbate ADD/ADHD symptomology, or which alleviate ADD/ADHD symptomology when removed, in patients diagnosed with or suspected of having ADD/ADHD. The test kit includes a plurality of distinct food preparations coupled to individually addressable respective solid carriers. The plurality of distinct food preparations have an average discriminatory p-value of ≤0.07 as determined by raw p-value or an average discriminatory p-value of ≤0.10 as determined by FDR multiplicity adjusted p-value. In some embodiments, the average discriminatory p-value is determined by a process, which includes comparing assay values of a first patient test cohort that is diagnosed with or suspected of having ADD/ADHD with assay values of a second patient test cohort that is not diagnosed with or suspected of having ADD/ADHD.

Another aspect of the embodiments described herein includes a method of identifying food items that trigger and/or exacerbate ADD/ADHD symptomology, or which alleviate ADD/ADHD symptomology when removed, in patients diagnosed with or suspected of having ADD/ADHD. The method includes a step of contacting a food preparation with a bodily fluid of a patient that is diagnosed with or suspected to have ADD/ADHD. The bodily fluid is associated with gender identification. In certain embodiments, the step of contacting is performed under conditions that allow IgG from the bodily fluid to bind to at least one component of the food preparation. The method continues with a step of measuring IgG bound to the at least one component of the food preparation to obtain a signal, and then comparing the signal to a gender-stratified reference value for the food preparation using the gender identification to obtain a result. Then, the method also includes a step of updating or generating a report using the result.

Another aspect of the embodiments described herein includes a method of generating a test for identifying of food items that trigger and/or exacerbate ADD/ADHD symptomology, or which alleviate ADD/ADHD symptomology when removed, in patients diagnosed with or suspected to have ADD/ADHD. The method includes a step of obtaining test results for a plurality of distinct food preparations. The test results are based on bodily fluids of patients diagnosed with or suspected to have ADD/ADHD and bodily fluids of a control group not diagnosed with or not suspected to have ADD/ADHD. The method also includes a step of stratifying the test results by gender for each of the distinct food preparations. Then the method continues with a step of assigning for a predetermined percentile rank a different cutoff value for male and female patients for each of the distinct food preparations.

Still another aspect of the embodiments described herein includes a use of a plurality of distinct food preparations coupled to individually addressable respective solid carriers in a diagnosis of ADD/ADHD. The plurality of distinct food preparations are selected based on their average discriminatory p-value of ≤0.07 as determined by raw p-value or an average discriminatory p-value of ≤0.10 as determined by FDR multiplicity adjusted p-value.

In one aspect, the disclosure features a test kit for testing food sensitivity in a patient diagnosed with or suspected of having ADD/ADHD, comprising one or more distinct food preparations, wherein each food preparation is independently coupled to an individually addressable solid carrier; wherein the distinct food preparations have an average discriminatory p-value of ≤0.07 as determined by raw p-value, or an average discriminatory p-value of ≤0.10 as determined by FDR multiplicity adjusted p-value. In some embodiments, the average discriminatory p-value is determined by a process comprising comparing assay values of a first patient test cohort that is diagnosed with or suspected of having ADD/ADHD with assay values of a second patient test cohort that is not diagnosed with or not suspected of having ADD/ADHD. In certain embodiments, the one or more distinct food preparations includes at least two food preparations prepared from food items of Table 1 or selected from foods 1-37 of Table 2. In certain embodiments, the one or more distinct food preparations includes at least three food preparations prepared from food items of Table 1 or selected from foods 1-37 of Table 2. In other embodiments, the one or more distinct food preparations includes at least four food preparations prepared from food items of Table 1 or selected from foods 1-37 of Table 2. In certain embodiments, the one or more distinct food preparations includes at least five food preparations prepared from food items of Table 1 or selected from foods 1-37 of Table 2. In certain embodiments, the one or more distinct food preparations includes at least six food preparations prepared from food items of Table 1 or selected from foods 1-37 of Table 2. In certain embodiments, the one or more distinct food preparations includes at least seven food preparations prepared from food items of Table 1 or selected from foods 1-37 of Table 2. In another embodiment, the one or more distinct food preparations includes at least eight food preparations prepared from food items of Table 1 or selected from foods 1-37 of Table 2. In certain embodiments, the one or more distinct food preparations includes at least nine food preparations prepared from food items of Table 1 or selected from foods 1-37 of Table 2. In certain embodiments, the one or more distinct food preparations includes at least ten food preparations prepared from food items of Table 1 or selected from foods 1-37 of Table 2. In certain embodiments, the one or more distinct food preparations includes at least eleven food preparations prepared from food items of Table 1 or selected from foods 1-37 of Table 2. In yet other embodiments, the one or more distinct food preparations includes at least 12 food preparations prepared from food items of Table 1 or selected from foods 1-37 of Table 2. In certain embodiments, the one or more distinct food preparations includes at least 13 food preparations prepared from food items of Table 1 or selected from foods 1-37 of Table 2. In certain embodiments, the one or more distinct food preparations includes at least 14 or more food preparations prepared from food items of Table 1 or selected from foods 1-37 of Table 2.

In some embodiments, the one or more food preparations is selected from the group consisting of at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2.

In some embodiments of the disclosure, the one or more distinct food preparations is selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2. In other embodiments, the one or more distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In other embodiments of the disclosure, the one or more distinct food preparations has an average discriminatory p-value of ≤0.05 as determined by raw p-value or an average discriminatory p-value of ≤0.08 as determined by FDR multiplicity adjusted p-value. In other embodiments, the one or more distinct food preparations has an average discriminatory p-value of ≤0.025 as determined by raw p-value or an average discriminatory p-value of ≤0.07 as determined by FDR multiplicity adjusted p-value.

In other embodiments of the disclosure, the FDR multiplicity adjusted p-value is adjusted for at least one of age and gender. In another embodiment, the FDR multiplicity adjusted p-value is adjusted for age and gender.

In other embodiments of the disclosure, at least 50% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 55% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 60% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 65% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In another embodiment, at least 70% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 75% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 80% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 85% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 95% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 95% of the one or more distinct food preparations, when adjusted for a single gender, has an average discriminatory p-value of ≤0.07 as determined by raw p-value or an average discriminatory p-value of ≤0.10 as determined by FDR multiplicity adjusted p-value. In another embodiment, all of the one or more distinct food preparations, when adjusted for a single gender, have an average discriminatory p-value of ≤0.07 as determined by raw p-value or an average discriminatory p-value of ≤0.10 as determined by FDR multiplicity adjusted p-value.

In certain embodiments of the disclosure, the one or more distinct food preparations are crude filtered aqueous extracts. In other embodiments, the one or more distinct food preparations are processed aqueous extracts. In yet other embodiments, the one or more distinct food preparations are crude filtered aqueous extracts or processed aqueous extracts. In certain other embodiments, the one or more distinct food preparations are crude filtered aqueous extracts or processed aqueous extracts.

In certain embodiments of the disclosure, the solid carrier is a well of a multiwell plate, a bead, an electrical, a chemical sensor, a microchip or an adsorptive film.

In certain other embodiments, the disclosure features a method of testing food sensitivity in a patient diagnosed with or suspected of having ADD/ADHD, comprising contacting a test kit (or diagnostic apparatus) of the disclosure with a bodily fluid of a patient that is diagnosed with or suspected of having ADD/ADHD, wherein the step of contacting is performed under conditions that allow IgG from the bodily fluid to bind to at least one component of the food preparation; measuring IgG bound to the at least one component of the food preparation to obtain a signal; and updating or generating a report using the signal. In some embodiments, the bodily fluid of the patient is selected from the group consisting of whole blood, plasma, serum, saliva, urine and a fecal suspension. In other embodiments, the step of contacting the test kit is performed with a plurality of distinct food preparations.

In certain embodiments, the plurality of distinct food preparations is prepared from food items of Table 1 or selected from foods 1-37 of Table 2. In yet other embodiments, the plurality of distinct food preparations is selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2.

In other embodiments, the plurality of distinct food preparations has an average discriminatory p-value of ≤0.07 as determined by raw p-value or an average discriminatory p-value of ≤0.10 as determined by FDR multiplicity adjusted p-value. In yet other embodiments, the plurality of distinct food preparations has an average discriminatory p-value of ≤0.05 as determined by raw p-value or an average discriminatory p-value of ≤0.08 as determined by FDR multiplicity adjusted p-value. In still other embodiments, the plurality of distinct food preparations has an average discriminatory p-value of ≤0.025 as determined by raw p-value or an average discriminatory p-value of ≤0.07 as determined by FDR multiplicity adjusted p-value. In certain embodiments, all of the plurality of distinct food preparations has an average discriminatory p-value of ≤0.07 as determined by raw p-value or an average discriminatory p-value of ≤0.10 as determined by FDR multiplicity adjusted p-value.

In some embodiments, the food preparation is immobilized on a solid surface. In other embodiments the food preparation is immobilized on a solid surface, optionally in an addressable manner.

In another embodiment, the step of measuring IgG bound to the at least one component of the food preparation is performed via immunoassay test. In yet another embodiment, the method comprises comparing the signal to a gender-stratified reference value for the food preparation using gender identification to obtain a result, wherein the gender-stratified reference value for the food preparation is at least a 90th percentile value.

In another aspect, the disclosure features a method of generating a test for food sensitivity in a patient diagnosed with or suspected of having ADD/ADHD, comprising obtaining test results for a plurality of distinct food preparations, wherein the test results are based on bodily fluids of patients diagnosed with or suspected of having ADD/ADHD and bodily fluids of a control group not diagnosed with or not suspected of having ADD/ADHD; stratifying the test results by gender for each of the distinct food preparations; assigning for a predetermined percentile rank a different cutoff value for male and female patients for each of the distinct food preparations; selecting a plurality of distinct food preparations that each have a discriminatory p-value of ≤0.07 as determined by raw p-value or a discriminatory p-value of ≤0.10 as determined by FDR multiplicity adjusted p-value; and generating a test consisting essentially of the selected food preparations for food sensitivity in a patient diagnosed with or suspected of having ADD/ADHD.

In some embodiments, the test result is an ELISA result.

In other embodiments, the plurality of distinct food preparations includes at least two food preparations prepared from food items of Table 1 or selected from foods 1-37 of Table 2. In another embodiment, the plurality of distinct food preparations includes at least three food preparations prepared from food items of Table 1 or selected from foods 1-37 of Table 2. In another embodiment, the plurality of distinct food preparations includes at least four food preparations prepared from food items of Table 1 or selected from foods 1-37 of Table 2. In another embodiment, the plurality of distinct food preparations includes at least five food preparations prepared from food items of Table 1 or selected from foods 1-37 of Table 2. In another embodiment, the plurality of distinct food preparations includes at least six food preparations prepared from food items of Table 1 or selected from foods 1-37 of Table 2. In another embodiment, the plurality of distinct food preparations includes at least seven food preparations prepared from food items of Table 1 or selected from foods 1-37 of Table 2. In another embodiment, the plurality of distinct food preparations includes at least eight food preparations prepared from food items of Table 1 or selected from foods 1-37 of Table 2. In another embodiment, the plurality of distinct food preparations includes at least nine food preparations prepared from food items of Table 1 or selected from foods 1-37 of Table 2. In another embodiment, the plurality of distinct food preparations includes at least ten food preparations prepared from food items of Table 1 or selected from foods 1-37 of Table 2. In another embodiment, the plurality of distinct food preparations includes at least eleven food preparations prepared from food items of Table 1 or selected from foods 1-37 of Table 2. In another embodiment, the plurality of distinct food preparations includes at least twelve food preparations prepared from food items of Table 1 or selected from foods 1-37 of Table 2. In another embodiment, the plurality of distinct food preparations includes at least thirteen food preparations prepared from food items of Table 1 or selected from foods 1-37 of Table 2. In another embodiment, the plurality of distinct food preparations includes at least fourteen or more food preparations prepared from food items of Table 1 or selected from foods 1-37 of Table 2.

In another embodiment, the plurality of distinct food preparations is selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2. In yet another embodiment, the plurality of distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In other embodiments, the plurality of different food preparations has an average discriminatory p-value of ≤0.05 as determined by raw p-value or an average discriminatory p-value of ≤0.08 as determined by FDR multiplicity adjusted p-value. In another embodiment, the plurality of different food preparations has an average discriminatory p-value of ≤0.025 as determined by raw p-value or an average discriminatory p-value of ≤0.07 as determined by FDR multiplicity adjusted p-value.

In certain embodiments, the bodily fluid of the patient is selected from the group consisting of whole blood, plasma, serum, saliva, urine or a fecal suspension.

In other embodiments, the predetermined percentile rank is an at least 90th percentile rank.

In yet another embodiment, the cutoff value for male and female patients has a difference of at least 10% (abs).

In a further embodiment, the method further comprises a step of normalizing the result to a patient's total IgG. In another embodiment, the method further comprises a step of normalizing the result to a global mean of the patient's food specific IgG results.

In other embodiments, the method further comprises a step of identifying a subset of patients, wherein the subset of patients' sensitivities to the food preparations underlies ADD/ADHD by raw p-value or an average discriminatory p-value of ≤0.01. In other embodiments, the method further comprises a step of determining numbers of the food preparations, wherein the numbers of the food preparations can be used to confirm ADD/ADHD by raw p-value or an average discriminatory p-value of ≤0.01.

In certain aspects, the disclosure features a use of a plurality of distinct food preparations coupled to individually addressable respective solid carriers for the diagnosis of ADD/ADHD, wherein the plurality of distinct food preparations have an average discriminatory p-value of ≤0.07 as determined by raw p-value or an average discriminatory p-value of ≤0.10 as determined by FDR multiplicity adjusted p-value. In certain embodiments, the plurality of food preparations includes at least two food preparations prepared from food items of Table 1 or selected from foods 1-37 of Table 2. In another embodiment, the plurality of distinct food preparations includes at least three food preparations prepared from food items of Table 1 or selected from foods 1-37 of Table 2. In another embodiment, the plurality of distinct food preparations includes at least four food preparations prepared from food items of Table 1 or selected from foods 1-37 of Table 2. In another embodiment, the plurality of distinct food preparations includes at least five food preparations prepared from food items of Table 1 or selected from foods 1-37 of Table 2. In another embodiment, the plurality of distinct food preparations includes at least six food preparations prepared from food items of Table 1 or selected from foods 1-37 of Table 2. In another embodiment, the plurality of distinct food preparations includes at least seven food preparations prepared from food items of Table 1 or selected from foods 1-37 of Table 2. In another embodiment, the plurality of distinct food preparations includes at least eight food preparations prepared from food items of Table 1 or selected from foods 1-37 of Table 2. In another embodiment, the plurality of distinct food preparations includes at least nine food preparations prepared from food items of Table 1 or selected from foods 1-37 of Table 2. In another embodiment, the plurality of distinct food preparations includes at least ten food preparations prepared from food items of Table 1 or selected from foods 1-37 of Table 2. In another embodiment, the plurality of distinct food preparations includes at least eleven food preparations prepared from food items of Table 1 or selected from foods 1-37 of Table 2. In another embodiment, the plurality of distinct food preparations includes at least twelve food preparations prepared from food items of Table 1 or selected from foods 1-37 of Table 2. In another embodiment, the plurality of distinct food preparations includes at least thirteen food preparations prepared from food items of Table 1 or selected from foods 1-37 of Table 2. In another embodiment, the plurality of distinct food preparations includes at least fourteen or more food preparations prepared from food items of Table 1 or selected from foods 1-37 of Table 2.

In other embodiments, the plurality of food preparations is selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2. In yet another embodiment, the plurality of food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In certain embodiments, the plurality of distinct food preparations has an average discriminatory p-value of ≤0.05 as determined by raw p-value or an average discriminatory p-value of ≤0.08 as determined by FDR multiplicity adjusted p-value. In other embodiments, the plurality of distinct food preparations has an average discriminatory p-value of ≤0.025 as determined by raw p-value or an average discriminatory p-value of ≤0.07 as determined by FDR multiplicity adjusted p-value.

In certain other embodiments, the FDR multiplicity adjusted p-value is adjusted for at least one of age and gender. In other embodiments, the FDR multiplicity adjusted p-value is adjusted for age and gender.

In other embodiments, at least 50% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 55% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 60% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 65% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet another embodiment, at least 70% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 75% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 80% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 85% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 90% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 95% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet another embodiment, all of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value.

In another embodiment, the plurality of distinct food preparations is crude filtered aqueous extracts. In other embodiments, the plurality of distinct food preparations is processed aqueous extracts. In yet other embodiments, the plurality of distinct food preparations is crude filtered aqueous extracts and processed aqueous extracts.

In another embodiment, the solid carrier is a well of a multiwell plate, a bead, an electrical sensor, a chemical sensor, a microchip, or an adsorptive film.

In other embodiments, the average discriminatory p-value is determined by a process comprising comparing assay values of a first patient test cohort that is diagnosed with or suspected of having ADD/ADHD headaches with assay values of a second patient test cohort that is not diagnosed with or not suspected of having ADD/ADHD headaches.

In yet another embodiment, the test result is an ELISA result derived from a process that includes separately contacting each distinct food preparation with the bodily fluid of each patient.

In certain aspects, the disclosure features a detection apparatus for testing food sensitivity in a patient diagnosed with or suspected of having ADD/ADHD, comprising a plurality of distinct food preparations, wherein each food preparation is independently coupled to an individually addressable solid carrier; wherein the plurality of distinct food preparations consists essentially of food preparations that each have a discriminatory p-value of ≤0.07 as determined by raw p-value, or a discriminatory p-value of ≤0.10 as determined by FDR multiplicity adjusted p-value. In other embodiments, the discriminatory p-value is determined by a process comprising comparing assay values of a first patient test cohort that is diagnosed with or suspected of having ADD/ADHD with assay values of a second patient test cohort that is not diagnosed with or not suspected of having ADD/ADHD.

In some embodiments, the plurality of food preparations includes at least two food preparations selected from food items of Table 1 or selected from foods items 1-37 of Table 2. In another embodiment, the plurality of distinct food preparations includes at least three food preparations prepared from food items of Table 1 or selected from foods 1-37 of Table 2. In another embodiment, the plurality of distinct food preparations includes at least four food preparations prepared from food items of Table 1 or selected from foods 1-37 of Table 2. In another embodiment, the plurality of distinct food preparations includes at least five food preparations prepared from food items of Table 1 or selected from foods 1-37 of Table 2. In another embodiment, the plurality of distinct food preparations includes at least six food preparations prepared from food items of Table 1 or selected from foods 1-37 of Table 2. In another embodiment, the plurality of distinct food preparations includes at least seven food preparations prepared from food items of Table 1 or selected from foods 1-37 of Table 2. In another embodiment, the plurality of distinct food preparations includes at least eight food preparations prepared from food items of Table 1 or selected from foods 1-37 of Table 2. In another embodiment, the plurality of distinct food preparations includes at least nine food preparations prepared from food items of Table 1 or selected from foods 1-37 of Table 2. In another embodiment, the plurality of distinct food preparations includes at least ten food preparations prepared from food items of Table 1 or selected from foods 1-37 of Table 2. In another embodiment, the plurality of distinct food preparations includes at least eleven food preparations prepared from food items of Table 1 or selected from foods 1-37 of Table 2. In another embodiment, the plurality of distinct food preparations includes at least twelve food preparations prepared from food items of Table 1 or selected from foods 1-37 of Table 2. In another embodiment, the plurality of distinct food preparations includes at least thirteen food preparations prepared from food items of Table 1 or selected from foods 1-37 of Table 2. In another embodiment, the plurality of distinct food preparations includes at least fourteen or more food preparations prepared from food items of Table 1 or selected from foods 1-37 of Table 2.

In other embodiments, the plurality of food preparations is selected from the group consisting of at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2. In yet other embodiments, the plurality of food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In some embodiments, the plurality of distinct food preparations consists essentially of food preparations that each has a discriminatory p-value of ≤0.05 as determined by raw p-value or a discriminatory p-value of ≤0.08 as determined by FDR multiplicity adjusted p-value.

In other embodiments, the FDR multiplicity adjusted p-value is adjusted for at least one of age or gender.

In yet other embodiments, at least 50% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 55% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 60% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 65% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 70% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 75% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 80% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 85% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 90% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 95% or more of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value.

In other embodiments, the plurality of distinct food preparations is a crude aqueous extract. In other embodiments, the plurality of distinct food preparations is a processed aqueous extract. In other embodiments, the plurality of distinct food preparations is a crude aqueous extract or a processed aqueous extract.

In certain aspects, the disclosure features a detection apparatus for testing food sensitivity in a patient diagnosed with or suspected of having ADD/ADHD, comprising a plurality of distinct food preparations, wherein each food preparation is independently coupled to an individually addressable solid carrier; wherein the plurality of distinct food preparations consists essentially of food preparations having an average discriminatory p-value of ≤0.07 as determined by raw p-value, or an average discriminatory p-value of ≤0.10 as determined by FDR multiplicity adjusted p-value. In some embodiments, the average discriminatory p-value is determined by a process comprising comparing assay values of a first patient test cohort that is diagnosed with or suspected of having ADD/ADHD with assay values of a second patient test cohort that is not diagnosed with or suspected of having ADD/ADHD.

In other embodiments, the plurality of food preparations includes at least two food preparations selected from food items of Table 1 or selected from foods items 1-37 of Table 2. In another embodiment, the plurality of distinct food preparations includes at least three food preparations prepared from food items of Table 1 or selected from foods 1-37 of Table 2. In another embodiment, the plurality of distinct food preparations includes at least four food preparations prepared from food items of Table 1 or selected from foods 1-37 of Table 2. In another embodiment, the plurality of distinct food preparations includes at least five food preparations prepared from food items of Table 1 or selected from foods 1-37 of Table 2. In another embodiment, the plurality of distinct food preparations includes at least six food preparations prepared from food items of Table 1 or selected from foods 1-37 of Table 2. In another embodiment, the plurality of distinct food preparations includes at least seven food preparations prepared from food items of Table 1 or selected from foods 1-37 of Table 2. In another embodiment, the plurality of distinct food preparations includes at least eight food preparations prepared from food items of Table 1 or selected from foods 1-37 of Table 2. In another embodiment, the plurality of distinct food preparations includes at least nine food preparations prepared from food items of Table 1 or selected from foods 1-37 of Table 2. In another embodiment, the plurality of distinct food preparations includes at least ten food preparations prepared from food items of Table 1 or selected from foods 1-37 of Table 2. In another embodiment, the plurality of distinct food preparations includes at least eleven food preparations prepared from food items of Table 1 or selected from foods 1-37 of Table 2. In another embodiment, the plurality of distinct food preparations includes at least twelve food preparations prepared from food items of Table 1 or selected from foods 1-37 of Table 2. In another embodiment, the plurality of distinct food preparations includes at least thirteen food preparations prepared from food items of Table 1 or selected from foods 1-37 of Table 2. In another embodiment, the plurality of distinct food preparations includes at least fourteen or more food preparations prepared from food items of Table 1 or selected from foods 1-37 of Table 2.

In other embodiments, the plurality of food preparations is selected from the group consisting of at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2. In another embodiment, the plurality of food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In certain other embodiment, the plurality of distinct food preparations consists essentially of food preparations having an average discriminatory p-value of ≤0.05 as determined by raw p-value or an average discriminatory p-value of ≤0.08 as determined by FDR multiplicity adjusted p-value.

In yet other embodiments, the FDR multiplicity adjusted p-value is adjusted for at least one of age or gender.

In yet other embodiments, at least 50% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 55% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In another embodiment, at least 60% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 65% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 70% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet another embodiment, at least 75% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In certain embodiments, at least 80% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In another embodiment, at least 85% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value.

In yet another embodiment, the plurality of distinct food preparations is a crude aqueous extract. In another embodiment, the plurality of distinct food preparations is a processed aqueous extract. In yet another embodiment, the plurality of distinct food preparations is a crude aqueous extract or a processed aqueous extract.

In certain aspects, the disclosure features a detection apparatus for testing food sensitivity in a patient diagnosed with or suspected of having ADD/ADHD, comprising a plurality of distinct food preparations, wherein each food preparation is independently coupled to an individually addressable solid carrier; wherein at least 50% of the plurality of distinct food preparations each has a discriminatory p-value of ≤0.07 as determined by raw p-value, or a discriminatory p-value of ≤0.10 as determined by FDR multiplicity adjusted p-value.

In one embodiment, at least 50% of the plurality of distinct food preparations each has a discriminatory p-value of ≤0.07 as determined by raw p-value, or a discriminatory p-value of ≤0.10 as determined by FDR multiplicity adjusted p-value. In another embodiment, at least 55% of the plurality of distinct food preparations each has a discriminatory p-value of ≤0.07 as determined by raw p-value, or a discriminatory p-value of ≤0.10 as determined by FDR multiplicity adjusted p-value. In yet another embodiment, at least 60% of the plurality of distinct food preparations each has a discriminatory p-value of ≤0.07 as determined by raw p-value, or a discriminatory p-value of ≤0.10 as determined by FDR multiplicity adjusted p-value. In other embodiments, at least 65% of the plurality of distinct food preparations each has a discriminatory p-value of ≤0.07 as determined by raw p-value, or a discriminatory p-value of ≤0.10 as determined by FDR multiplicity adjusted p-value. In another embodiment, at least 70% of the plurality of distinct food preparations each has a discriminatory p-value of ≤0.07 as determined by raw p-value, or a discriminatory p-value of ≤0.10 as determined by FDR multiplicity adjusted p-value. In other embodiments, at least 75% of the plurality of distinct food preparations each has a discriminatory p-value of ≤0.07 as determined by raw p-value, or a discriminatory p-value of ≤0.10 as determined by FDR multiplicity adjusted p-value. In certain embodiments, at least 80% of the plurality of distinct food preparations each has a discriminatory p-value of ≤0.07 as determined by raw p-value, or a discriminatory p-value of ≤0.10 as determined by FDR multiplicity adjusted p-value. In another embodiment, at least 85% of the plurality of distinct food preparations each has a discriminatory p-value of ≤0.07 as determined by raw p-value, or a discriminatory p-value of ≤0.10 as determined by FDR multiplicity adjusted p-value. In other embodiments, at least 90% of the plurality of distinct food preparations each has a discriminatory p-value of ≤0.07 as determined by raw p-value, or a discriminatory p-value of ≤0.10 as determined by FDR multiplicity adjusted p-value. In other embodiments, at least 95% of the plurality of distinct food preparations each has a discriminatory p-value of ≤0.07 as determined by raw p-value, or a discriminatory p-value of ≤0.10 as determined by FDR multiplicity adjusted p-value.

In some embodiments, the plurality of food preparations is selected from the group consisting of at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, and at least 37 food preparations from foods 1-37 of Table 2.

In some embodiments, the plurality of distinct food preparations has an average discriminatory p-value of ≤0.05 as determined by raw p-value or an average discriminatory p-value of ≤0.08 as determined by FDR multiplicity adjusted p-value.

In other embodiments, the FDR multiplicity adjusted p-value is adjusted for at least one of age or gender.

In yet other embodiments, at least 50% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In other embodiments, at least 55% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 60% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In another embodiment, at least 65% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 70% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 75% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 80% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 85% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 90% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value. In yet other embodiments, at least 95% of the plurality of distinct food preparations, when adjusted for a single gender, has an average discriminatory p-value of ≤0.07 as determined by raw p-value or an average discriminatory p-value of ≤0.10 as determined by FDR multiplicity adjusted p-value.

In some embodiments, the plurality of distinct food preparations is crude aqueous. In other embodiments, the plurality of distinct food preparations is processed aqueous extracts. In some embodiments, the plurality of distinct food preparations is crude aqueous extracts or processed aqueous extracts.

In certain aspects, the disclosure features a detection apparatus for testing food sensitivity in a biological sample from a patient diagnosed with or suspected of having ADD/ADHD, comprising a solid surface with a plurality of food preparations, wherein each food preparation is independently coupled to the solid surface, wherein the plurality of food preparations consists essentially of trigger foods for ADD/ADHD capable of reacting with an immunoglobulin from the biological sample. In certain embodiments, the trigger foods is selected from the group consisting of at least 2 food items from foods 1-37 of Table 2, at least 3 food items from foods 1-37 of Table 2, at least 4 food items from foods 1-37 of Table 2, at least 5 food items from foods 1-37 of Table 2, at least 6 food items from foods 1-37 of Table 2, at least 7 food items from foods 1-37 of Table 2, at least 8 food items from foods 1-37 of Table 2, at least 9 food items from foods 1-37 of Table 2, at least 10 food items from foods 1-37 of Table 2, at least 11 food items from foods 1-37 of Table 2, at least 12 food items from foods 1-37 of Table 2, at least 13 food items from foods 1-37 of Table 2, at least 14 food items from foods 1-37 of Table 2, at least 15 food items from foods 1-37 of Table 2, at least 16 food items from foods 1-37 of Table 2, at least 17 food items from foods 1-37 of Table 2, at least 18 food items from foods 1-37 of Table 2, at least 19 food items from foods 1-37 of Table 2, at least 20 food items from foods 1-37 of Table 2, at least 21 food items from foods 1-37 of Table 2, at least 22 food items from foods 1-37 of Table 2, at least 23 food items from foods 1-37 of Table 2, at least 24 food items from foods 1-37 of Table 2, at least 25 food items from foods 1-37 of Table 2, at least 26 food items from foods 1-37 of Table 2, at least 27 food items from foods 1-37 of Table 2, at least 28 food items from foods 1-37 of Table 2, at least 29 food items from foods 1-37 of Table 2, at least 30 food items from foods 1-37 of Table 2, at least 31 food items from foods 1-37 of Table 2, at least 32 food items from foods 1-37 of Table 2, at least 33 food items from foods 1-37 of Table 2, at least 34 food items from foods 1-37 of Table 2, at least 35 food items from foods 1-37 of Table 2, at least 36 food items from foods 1-37 of Table 2, and at least 37 food items from foods 1-37 of Table 2. In other embodiments, the trigger foods is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In some embodiments, the plurality of distinct food preparations is crude aqueous. In other embodiments, the plurality of distinct food preparations is processed aqueous extracts. In some embodiments, the plurality of distinct food preparations is crude aqueous extracts or processed aqueous extracts.

In certain aspects, the disclosure features a detection apparatus for testing food sensitivity in a biological sample from a patient diagnosed with or suspected of having ADD/ADHD, comprising a solid surface with a plurality of food preparations, wherein each food preparation is independently coupled to the solid surface, wherein at least 50% of the plurality of food preparations is a trigger food for ADD/ADHD capable of reacting with an immunoglobulin from the biological sample. In another embodiment, at least 55% of the plurality of food preparations is a trigger food for ADD/ADHD capable of reacting with an immunoglobulin from the biological sample. In another embodiment, at least 60% of the plurality of food preparations is a trigger food for ADD/ADHD capable of reacting with an immunoglobulin from the biological sample. In other embodiments, at least 65% of the plurality of food preparations is a trigger food for ADD/ADHD capable of reacting with an immunoglobulin from the biological sample. In yet another embodiment, at least 70% of the plurality of food preparations is a trigger food for ADD/ADHD capable of reacting with an immunoglobulin from the biological sample. In further embodiments, at least 75% of the plurality of food preparations is a trigger food for ADD/ADHD capable of reacting with an immunoglobulin from the biological sample. In another embodiment, at least 80% of the plurality of food preparations is a trigger food for ADD/ADHD capable of reacting with an immunoglobulin from the biological sample. In certain other embodiments, at least 85% of the plurality of food preparations is a trigger food for ADD/ADHD capable of reacting with an immunoglobulin from the biological sample. In other embodiments, at least 90% of the plurality of food preparations is a trigger food for ADD/ADHD capable of reacting with an immunoglobulin from the biological sample. In yet other embodiments, at least 95% of the plurality of food preparations is a trigger food for ADD/ADHD capable of reacting with an immunoglobulin from the biological sample.

In some embodiments, the trigger foods is selected from the group consisting of at least 2 food items from foods 1-37 of Table 2, at least 3 food items from foods 1-37 of Table 2, at least 4 food items from foods 1-37 of Table 2, at least 5 food items from foods 1-37 of Table 2, at least 6 food items from foods 1-37 of Table 2, at least 7 food items from foods 1-37 of Table 2, at least 8 food items from foods 1-37 of Table 2, at least 9 food items from foods 1-37 of Table 2, at least 10 food items from foods 1-37 of Table 2, at least 11 food items from foods 1-37 of Table 2, at least 12 food items from foods 1-37 of Table 2, at least 13 food items from foods 1-37 of Table 2, at least 14 food items from foods 1-37 of Table 2, at least 15 food items from foods 1-37 of Table 2, at least 16 food items from foods 1-37 of Table 2, at least 17 food items from foods 1-37 of Table 2, at least 18 food items from foods 1-37 of Table 2, at least 19 food items from foods 1-37 of Table 2, at least 20 food items from foods 1-37 of Table 2, at least 21 food items from foods 1-37 of Table 2, at least 22 food items from foods 1-37 of Table 2, at least 23 food items from foods 1-37 of Table 2, at least 24 food items from foods 1-37 of Table 2, at least 25 food items from foods 1-37 of Table 2, at least 26 food items from foods 1-37 of Table 2, at least 27 food items from foods 1-37 of Table 2, at least 28 food items from foods 1-37 of Table 2, at least 29 food items from foods 1-37 of Table 2, at least 30 food items from foods 1-37 of Table 2, at least 31 food items from foods 1-37 of Table 2, at least 32 food items from foods 1-37 of Table 2, at least 33 food items from foods 1-37 of Table 2, at least 34 food items from foods 1-37 of Table 2, at least 35 food items from foods 1-37 of Table 2, at least 36 food items from foods 1-37 of Table 2, and at least 37 food items from foods 1-37 of Table 2. In yet another embodiment, the trigger foods is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In some embodiments, the plurality of distinct food preparations is crude aqueous. In other embodiments, the plurality of distinct food preparations is processed aqueous extracts. In some embodiments, the plurality of distinct food preparations is crude aqueous extracts or processed aqueous extracts.

In certain aspects, the disclosure features a detection apparatus for testing food sensitivity in a biological sample from a patient diagnosed with or suspected of having ADD/ADHD, comprising a solid surface with a plurality of food preparations, wherein each food preparation is independently coupled to the solid surface, wherein at least seven food preparations of the plurality of food preparations is a trigger food for ADD/ADHD capable of reacting with an immunoglobulin from the biological sample. In other embodiments, at least eight food preparations of the plurality of food preparations is a trigger food for ADD/ADHD capable of reacting with an immunoglobulin from the biological sample. In other embodiments, at least nine food preparations of the plurality of food preparations is a trigger food for ADD/ADHD capable of reacting with an immunoglobulin from the biological sample. In other embodiments, at least ten food preparations of the plurality of food preparations is a trigger food for ADD/ADHD capable of reacting with an immunoglobulin from the biological sample. In other embodiments, at least eleven food preparations of the plurality of food preparations is a trigger food for ADD/ADHD capable of reacting with an immunoglobulin from the biological sample. In other embodiments, at least twelve food preparations of the plurality of food preparations is a trigger food for ADD/ADHD capable of reacting with an immunoglobulin from the biological sample. In other embodiments, at least thirteen food preparations of the plurality of food preparations is a trigger food for ADD/ADHD capable of reacting with an immunoglobulin from the biological sample. In other embodiments, at least fourteen food preparations of the plurality of food preparations is a trigger food for ADD/ADHD capable of reacting with an immunoglobulin from the biological sample.

In other embodiments, the trigger foods is selected from the group consisting of at least 2 food items from foods 1-37 of Table 2, at least 3 food items from foods 1-37 of Table 2, at least 4 food items from foods 1-37 of Table 2, at least 5 food items from foods 1-37 of Table 2, at least 6 food items from foods 1-37 of Table 2, at least 7 food items from foods 1-37 of Table 2, at least 8 food items from foods 1-37 of Table 2, at least 9 food items from foods 1-37 of Table 2, at least 10 food items from foods 1-37 of Table 2, at least 11 food items from foods 1-37 of Table 2, at least 12 food items from foods 1-37 of Table 2, at least 13 food items from foods 1-37 of Table 2, at least 14 food items from foods 1-37 of Table 2, at least 15 food items from foods 1-37 of Table 2, at least 16 food items from foods 1-37 of Table 2, at least 17 food items from foods 1-37 of Table 2, at least 18 food items from foods 1-37 of Table 2, at least 19 food items from foods 1-37 of Table 2, at least 20 food items from foods 1-37 of Table 2, at least 21 food items from foods 1-37 of Table 2, at least 22 food items from foods 1-37 of Table 2, at least 23 food items from foods 1-37 of Table 2, at least 24 food items from foods 1-37 of Table 2, at least 25 food items from foods 1-37 of Table 2, at least 26 food items from foods 1-37 of Table 2, at least 27 food items from foods 1-37 of Table 2, at least 28 food items from foods 1-37 of Table 2, at least 29 food items from foods 1-37 of Table 2, at least 30 food items from foods 1-37 of Table 2, at least 31 food items from foods 1-37 of Table 2, at least 32 food items from foods 1-37 of Table 2, at least 33 food items from foods 1-37 of Table 2, at least 34 food items from foods 1-37 of Table 2, at least 35 food items from foods 1-37 of Table 2, at least 36 food items from foods 1-37 of Table 2, and at least 37 food items from foods 1-37 of Table 2. In yet other embodiments, the trigger foods is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In some embodiments, the plurality of distinct food preparations is crude aqueous. In other embodiments, the plurality of distinct food preparations is processed aqueous extracts. In some embodiments, the plurality of distinct food preparations is crude aqueous extracts or processed aqueous extracts.

In certain aspects, the disclosure features a test kit with one or more distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In certain aspects, the disclosure features methods using one or more distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In certain aspects, the disclosure features uses using one or more distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

In certain aspects, the disclosure features a detection apparatus with one or more distinct food preparations is selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

Various objects, features, aspects and advantages of the embodiments described herein will become more apparent from the following detailed description of the embodiments, along with the accompanying drawing figures in which like numerals represent like components.

### BRIEF DESCRIPTION OF THE DRAWINGS

Table 1 shows a list of food items from which food preparations can be prepared.

Table 2 shows statistical data of foods ranked according to 2-tailed FDR multiplicity-adjusted p-values.

Table 3 shows statistical data of ELISA score by food and gender.

Table 4 shows cutoff values of foods for a predetermined percentile rank.

FIG. 1A illustrates ELISA signal score of male ADD/ADHD patients and control tested with cantaloupe.

FIG. 1B illustrates a distribution of percentage of male ADD/ADHD subjects exceeding the 90^{th} and 95^{th} percentile tested with cantaloupe.

FIG. 1C illustrates a signal distribution in women along with the 95^{th} percentile cutoff as determined from the female control population tested with cantaloupe.

FIG. 1D illustrates a distribution of percentage of female ADD/ADHD subjects exceeding the 90^{th} and 95^{th} percentile tested with cantaloupe.

FIG. 2A illustrates ELISA signal score of male ADD/ADHD patients and control tested with wheat.

FIG. 2B illustrates a distribution of percentage of male ADD/ADHD subjects exceeding the 90^{th} and 95^{th} percentile tested with wheat.

FIG. 2C illustrates a signal distribution in women along with the 95^{th} percentile cutoff as determined from the female control population tested with wheat.

FIG. 2D illustrates a distribution of percentage of female ADD/ADHD subjects exceeding the 90^{th} and 95^{th} percentile tested with wheat.

FIG. 3A illustrates ELISA signal score of male ADD/ADHD patients and control tested with tomato.

FIG. 3B illustrates a distribution of percentage of male ADD/ADHD subjects exceeding the 90^{th} and 95^{th} percentile tested with tomato.

FIG. 3C illustrates a signal distribution in women along with the 95^{th} percentile cutoff as determined from the female control population tested with tomato.

FIG. 3D illustrates a distribution of percentage of female ADD/ADHD subjects exceeding the 90^{th} and 95^{th} percentile tested with tomato.

FIG. 4A illustrates ELISA signal score of male ADD/ADHD patients and control tested with cucumber.

FIG. 4B illustrates a distribution of percentage of male ADD/ADHD subjects exceeding the 90^{th} and 95^{th} percentile tested with cucumber.

FIG. 4C illustrates a signal distribution in women along with the 95^{th} percentile cutoff as determined from the female control population tested with cucumber.

FIG. 4D illustrates a distribution of percentage of female ADD/ADHD subjects exceeding the 90^{th} and 95^{th} percentile tested with cucumber.

FIG. 5A illustrates distributions of ADD/ADHD subjects by number of foods that were identified as trigger foods at the 90^{th} percentile.

FIG. 5B illustrates distributions of ADD/ADHD subjects by number of foods that were identified as trigger foods at the 95^{th} percentile.

Table 5A shows raw data of ADD/ADHD patients and control with number of positive results based on the 90^{th} percentile.

Table 5B shows raw data of ADD/ADHD patients and control with number of positive results based on the 95^{th} percentile.

Table 6A shows statistical data summarizing the raw data of ADD/ADHD patient populations shown in Table 5A.

Table 6B shows statistical data summarizing the raw data of ADD/ADHD patient populations shown in Table 5B.

Table 7A shows statistical data summarizing the raw data of control populations shown in Table 5A.

Table 7B shows statistical data summarizing the raw data of control populations shown in Table 5B.

Table 8A shows statistical data summarizing the raw data of ADD/ADHD patient populations shown in Table 5A transformed by logarithmic transformation.

Table 8B shows statistical data summarizing the raw data of ADD/ADHD patient populations shown in Table 5B transformed by logarithmic transformation.

Table 9A shows statistical data summarizing the raw data of control populations shown in Table 5A transformed by logarithmic transformation.

Table 9B shows statistical data summarizing the raw data of control populations shown in Table 5B transformed by logarithmic transformation.

Table 10A shows statistical data of an independent T-test to compare the geometric mean number of positive foods between the ADD/ADHD and non-ADD/ADHD samples based on the 90^{th} percentile.

Table 10B shows statistical data of an independent T-test to compare the geometric mean number of positive foods between the ADD/ADHD and non-ADD/ADHD samples based on the 95^{th} percentile.

Table 11A shows statistical data of a Mann-Whitney test to compare the geometric mean number of positive foods between the ADD/ADHD and non-ADD/ADHD samples based on the 90^{th} percentile.

Table 11B shows statistical data of a Mann-Whitney test to compare the geometric mean number of positive foods between the ADD/ADHD and non-ADD/ADHD samples based on the 95^{th} percentile.

FIG. 6A illustrates a box and whisker plot of data shown in Table 5A.

FIG. 6B illustrates a notched box and whisker plot of data shown in Table 5A.

FIG. 6C illustrates a box and whisker plot of data shown in Table 5B.

FIG. 6D illustrates a notched box and whisker plot of data shown in Table 5B.

Table 12A shows statistical data of a Receiver Operating Characteristic (ROC) curve analysis of data shown in Tables 5A-11A.

Table 12B shows statistical data of a Receiver Operating Characteristic (ROC) curve analysis of data shown in Tables 5B-11B.

FIG. 7A illustrates the ROC curve corresponding to the statistical data shown in Table 12A.

FIG. 7B illustrates the ROC curve corresponding to the statistical data shown in Table 12B.

Table 13A shows a statistical data of performance metrics in predicting ADD/ADHD status among female patients from number of positive foods based on the 90^{th} percentile.

Table 13B shows a statistical data of performance metrics in predicting ADD/ADHD status among male patients from number of positive foods based on the 90^{th} percentile.

Table 14A shows a statistical data of performance metrics in predicting ADD/ADHD status among female patients from number of positive foods based on the 95^{th} percentile.

Table 14B shows a statistical data of performance metrics in predicting ADD/ADHD status among male patients from number of positive foods based on the 95^{th} percentile.

### DETAILED DESCRIPTION

The inventors have discovered that food preparations used in food tests to identify trigger foods in patients diagnosed with or suspected of having ADD/ADHD are not equally well predictive and/or associated with ADD/ADHD symptoms. Indeed, various experiments have revealed that among a wide variety of food items, certain food items are highly predictive/associated with ADD/ADHD whereas others have no statistically significant association with ADD/ADHD.

Even more unexpectedly, the inventors discovered that in addition to the high variability of food items, gender variability with respect to response in a test plays a substantial role in the determination of association of a food item with ADD/ADHD. Consequently, based on the inventors' findings and further contemplations, test kits, detection apparatus or devices, array, chip or test panel and methods of using same are now presented with substantially higher predictive power in the choice of food items (i.e., trigger foods) that could be eliminated for reduction of ADD/ADHD signs and symptoms.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The term “average discriminatory p-value”, as used herein, generally refers to an average of all the p-values with a particular probability (e.g., ≤0.05) as determined by raw p-value or with a particular probability (e.g., ≤0.1) as determined by FDR multiplicity adjusted p-value that were identified by analytical methods described herein, e.g., the analytical methods that produced the results summarized in Table 2 (for example, and in particular, foods 1-37 in Table 2). In one embodiment, average discriminatory p-value refers to an average of all the p-values with a particular probability (e.g., ≤0.05) as determined by raw p-value or with a particular probability (e.g., ≤0.1) as determined by FDR multiplicity adjusted p-value that were identified by analytical methods described herein. In another embodiment, average discriminatory p-value refers to an average of all the p-values that are ≤0.1 as determined by raw p-value that were identified by analytical methods described herein. In another embodiment, average discriminatory p-value refers to an average of all the p-values that are ≤0.09 as determined by raw p-value that were identified by analytical methods described herein. In another embodiment, average discriminatory p-value refers to an average of all the p-values that are ≤0.08 as determined by raw p-value that were identified by analytical methods described herein. In another embodiment, average discriminatory p-value refers to an average of all the p-values that are ≤0.075 as determined by raw p-value that were identified by analytical methods described herein. In another embodiment, average discriminatory p-value refers to an average of all the p-values that are ≤0.07 as determined by raw p-value that were identified by analytical methods described herein. In another embodiment, average discriminatory p-value refers to an average of all the p-values that are ≤0.065 as determined by raw p-value that were identified by analytical methods described herein. In another embodiment, average discriminatory p-value refers to an average of all the p-values that are ≤0.05 as determined by raw p-value that were identified by analytical methods described herein. In another embodiment, average discriminatory p-value refers to an average of all the p-values that are ≤0.1 as determined by False Discovery Rate (FDR) multiplicity adjusted p-value that were identified by analytical methods described herein. In another embodiment, average discriminatory p-value refers to an average of all the p-values that are ≤0.09 as determined by False Discovery Rate (FDR) multiplicity adjusted p-value that were identified by analytical methods described herein. In another embodiment, average discriminatory p-value refers to an average of all the p-values that are ≤0.095 as determined by False Discovery Rate (FDR) multiplicity adjusted p-value that were identified by analytical methods described herein. In another embodiment, average discriminatory p-value refers to an average of all the p-values that are ≤0.09 as determined by False Discovery Rate (FDR) multiplicity adjusted p-value that were identified by analytical methods described herein. In another embodiment, average discriminatory p-value refers to an average of all the p-values that are ≤0.085 as determined by False Discovery Rate (FDR) multiplicity adjusted p-value that were identified by analytical methods described herein. In another embodiment, average discriminatory p-value refers to an average of all the p-values that are ≤0.08 as determined by False Discovery Rate (FDR) multiplicity adjusted p-value that were identified by analytical methods described herein. In another embodiment, average discriminatory p-value refers to an average of all the p-values that are ≤0.075 as determined by False Discovery Rate (FDR) multiplicity adjusted p-value that were identified by analytical methods described herein. In another embodiment, average discriminatory p-value refers to an average of all the p-values that are ≤0.07 as determined by False Discovery Rate (FDR) multiplicity adjusted p-value that were identified by analytical methods described herein. In another embodiment, average discriminatory p-value refers to an average of all the p-values that are ≤0.065 as determined by False Discovery Rate (FDR) multiplicity adjusted p-value that were identified by analytical methods described herein. In another embodiment, average discriminatory p-value refers to an average of all the p-values that are ≤0.06 as determined by False Discovery Rate (FDR) multiplicity adjusted p-value that were identified by analytical methods described herein.

The term “food preparation”, as used herein, refers to a solubilized aqueous extraction of a specific food item (e.g., cantaloupe, wheat, milk, etc.). In certain embodiments, the specific food item is solubilized using a blender or similar apparatus, in the presence of a buffer and the food item is processed until the structure of the food item is broken down into a homogenous liquid suspension or solution.

The term “individually addressable”, as used herein, refers to a portion of a solid carrier (e.g. an ELISA well, etc.), wherein a food preparation is immobilized (or coupled, etc.) to said portion of the solid carrier in a manner that separates said food preparation from other food preparations immobilized to the solid carrier, and that allows for the detection of an immunoglobulin (e.g., an IgG or other binding molecule) capable of binding to said food preparation (or a component thereof).

The term “one component of the food preparation” or “a component of the food preparation”, as used herein, refers to any portion of a food preparation (e.g., a protein(s), a lipid(s), a sugar(s), etc.) that is antigenic (i.e., capable of inducing and/or eliciting an immune response in a subject or patient).

The term “trigger food”, as used herein, broadly refers to a food preparation, or a component thereof, which will result in a significantly elevated immune response in a subject (e.g., a patient) exposed to the food preparation, or a component thereof, wherein the elevated immune response is highly correlated to the presence of a disease symptom(s), and which potentially may trigger and/or exacerbate a disease symptom(s); and/or which may potentially result in an alleviation or reduction of symptoms by removing the food preparation, or component thereof. In one embodiment, a trigger food may be characterized by a p-value of about ≤0.15 as determined by raw p-value. In another embodiment, a trigger food may be characterized by a p-value of about ≤0.10 as determined by raw p-value. In another embodiment, a trigger food may be characterized by a p-value of about ≤0.075 as determined by raw p-value. In yet another embodiment, a trigger food may be characterized by a p-value of about ≤0.05 as determined by raw p-value. In other embodiments, a trigger food may be characterized by a p-value of about ≤0.10 as determined by False Discovery Rate (FDR) multiplicity adjusted p-value. In another embodiment, a trigger food may be characterized by a p-value of about ≤0.09 as determined by False Discovery Rate (FDR) multiplicity adjusted p-value. In another embodiment, a trigger food may be characterized by a p-value of about ≤0.08 as determined by False Discovery Rate (FDR) multiplicity adjusted p-value. In yet another embodiment, a trigger food may be characterized by a p-value of about ≤0.07 as determined by False Discovery Rate (FDR) multiplicity adjusted p-value. In another embodiment, a trigger food may be characterized as one of the food preparations listed in Table 1 and/or Table 2. In another embodiment, a trigger food may be characterized as one of the food preparations listed in Table 2. In another embodiment, a trigger food may be characterized as one of the food preparations with a rank number of 1-37 (i.e., also referred to herein as “foods 1-37 of Table 2”) as described in Table 2.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of any embodiments of the disclosure otherwise claimed. No language in the specification should be construed as indicating any non-claimed element essential to the practice of any embodiments of the disclosure.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In one aspect, the inventors therefore contemplate a test kit, detection apparatus or device, array, chip or test panel that is suitable for identifying food items that trigger and/or exacerbate ADD/ADHD symptomology, or which alleviate ADD/ADHD symptomology when removed, in patients where the patient is diagnosed with or suspected of having ADD/ADHD. In some embodiments, such test kit, detection device or apparatus, array, chip or panel will include one or more (e.g., a plurality) of distinct food preparations (e.g., raw or processed extract, e.g., an aqueous extract with optional co-solvent, which may or may not be filtered) that are coupled to individually addressable respective solid carriers (e.g., in a form of an array or a micro well plate), wherein the distinct food preparations have an average discriminatory p-value of ≤0.07 as determined by raw p-value or an average discriminatory p-value of ≤0.10 as determined by FDR multiplicity adjusted p-value. In certain embodiments, the plurality of distinct food preparations consists essentially of food preparations that each has a discriminatory p-value of ≤0.07 as determined by raw p-value or a discriminatory p-value of ≤0.10 as determined by FDR multiplicity adjusted p-value. In certain embodiments, at least 50% of the plurality of distinct food preparations each has a discriminatory p-value of ≤0.07 as determined by raw p-value or a discriminatory p-value of ≤0.10 as determined by FDR multiplicity adjusted p-value.

In some embodiments, the numbers expressing quantities of ingredients, properties such as concentration, reaction conditions, and so forth, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

While not limiting to the inventive subject matter, food preparations will typically be drawn from foods generally known or suspected to trigger and/or exacerbate signs or symptoms of ADD/ADHD, or which alleviate ADD/ADHD symptomology when removed. Particularly suitable food preparations may be identified by the experimental procedures outlined below. Thus, it should be appreciated that the food items (e.g., trigger foods) need not be limited to the items described herein, but that all items are contemplated that can be identified by the methods presented herein. Therefore, in certain embodiments, exemplary food preparations include at least two, at least four, at least eight, or at least 12 food preparations prepared from foods 1-37 of Table 2. In other embodiments, exemplary food preparations are selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, at least 37 food preparations from foods 1-37 of Table 2. In other embodiments, exemplary food preparations are selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper. Still further especially contemplated food items and food additives from which food preparations can be prepared are listed in Table 1.

Using bodily fluids from patients diagnosed with or suspected of having ADD/ADHD and healthy control group individuals (i.e., those not diagnosed with or not suspected of having ADD/ADHD), numerous additional food items (i.e., trigger foods) may be identified. In one embodiment, identified food items (i.e., trigger foods) will have high discriminatory power and as such have a p-value of ≤0.15 as determined by raw p-value. In other embodiments, identified food items (i.e., trigger foods) will have high discriminatory power and as such have a p-value of ≤0.14 as determined by raw p-value. In other embodiments, identified food items (i.e., trigger foods) will have high discriminatory power and as such have a p-value of ≤0.13 as determined by raw p-value. In other embodiments, identified food items (i.e., trigger foods) will have high discriminatory power and as such have a p-value of ≤0.12 as determined by raw p-value. In other embodiments, identified food items (i.e., trigger foods) will have high discriminatory power and as such have a p-value of ≤0.11 as determined by raw p-value. In other embodiments, identified food items (i.e., trigger foods) will have high discriminatory power and as such have a p-value of ≤0.10 as determined by raw p-value. In other embodiments, identified food items (i.e., trigger foods) will have high discriminatory power and as such have a p-value of ≤0.09 as determined by raw p-value. In other embodiments, identified food items (i.e., trigger foods) will have high discriminatory power and as such have a p-value of ≤0.08 as determined by raw p-value. In other embodiments, identified food items (i.e., trigger foods) will have high discriminatory power and as such have a p-value of ≤0.07 as determined by raw p-value. In other embodiments, identified food items (i.e., trigger foods) will have high discriminatory power and as such have a p-value of ≤0.06 as determined by raw p-value. In yet other embodiments, identified food items (i.e., trigger foods) will have high discriminatory power and as such have a p-value of ≤0.05 as determined by raw p-value. In certain embodiments, identified food items (i.e., trigger foods) will have high discriminatory power and as such have a p-value of ≤0.10 as determined by False Discovery Rate (FDR) multiplicity adjusted p-value. In other embodiments, identified food items (i.e., trigger foods) will have high discriminatory power and as such have a p-value of ≤0.09 as determined by False Discovery Rate (FDR) multiplicity adjusted p-value. In other embodiments, identified food items (i.e., trigger foods) will have high discriminatory power and as such have a p-value of ≤0.08 as determined by False Discovery Rate (FDR) multiplicity adjusted p-value. In yet other embodiments, identified food items (i.e., trigger foods) will have high discriminatory power and as such have a p-value of ≤0.07 as determined by False Discovery Rate (FDR) multiplicity adjusted p-value.

In certain embodiments, such identified food preparations will have high discriminatory power and, as such, will have a p-value of ≤0.15, ≤0.10, or even ≤0.05 as determined by raw p-value, and/or a p-value of ≤0.10, ≤0.08, or even ≤0.07 as determined by False Discovery Rate (FDR) multiplicity adjusted p-value. In one embodiment, identified food preparations will have high discriminatory power and as such have a p-value of ≤0.15 as determined by raw p-value. In other embodiments, identified food preparations will have high discriminatory power and as such have a p-value of ≤0.14 as determined by raw p-value. In other embodiments, identified food preparations will have high discriminatory power and as such have a p-value of ≤0.13 as determined by raw p-value. In other embodiments, identified food preparations will have high discriminatory power and as such have a p-value of ≤0.12 as determined by raw p-value. In other embodiments, identified food preparations will have high discriminatory power and as such have a p-value of ≤0.11 as determined by raw p-value. In other embodiments, identified food preparations will have high discriminatory power and as such have a p-value of ≤0.10 as determined by raw p-value. In other embodiments, identified food preparations will have high discriminatory power and as such have a p-value of ≤0.09 as determined by raw p-value. In other embodiments, identified food preparations will have high discriminatory power and as such have a p-value of ≤0.08 as determined by raw p-value. In other embodiments, identified food preparations will have high discriminatory power and as such have a p-value of ≤0.07 as determined by raw p-value. In other embodiments, identified food preparations will have high discriminatory power and as such have a p-value of ≤0.06 as determined by raw p-value. In yet other embodiments, identified food preparations will have high discriminatory power and as such have a p-value of ≤0.05 as determined by raw p-value. In certain embodiments, identified food preparations will have high discriminatory power and as such have a p-value of ≤0.10 as determined by False Discovery Rate (FDR) multiplicity adjusted p-value. In other embodiments, identified food preparations will have high discriminatory power and as such have a p-value of ≤0.09 as determined by False Discovery Rate (FDR) multiplicity adjusted p-value. In other embodiments, identified food preparations will have high discriminatory power and as such have a p-value of ≤0.08 as determined by False Discovery Rate (FDR) multiplicity adjusted p-value. In yet other embodiments, identified food preparations will have high discriminatory power and as such have a p-value of ≤0.07 as determined by False Discovery Rate (FDR) multiplicity adjusted p-value.

Therefore, where a test kit, detection device or apparatus, array, chip, or panel has multiple (i.e., a plurality of) food preparations, it is contemplated that in certain embodiments, the plurality of distinct food preparations has an average discriminatory p-value of ≤0.05 as determined by raw p-value or an average discriminatory p-value of ≤0.08 as determined by FDR multiplicity adjusted p-value. In other embodiments, the plurality of distinct food preparations has an average discriminatory p-value of ≤0.025 as determined by raw p-value or an average discriminatory p-value of ≤0.07 as determined by FDR multiplicity adjusted p-value. In other embodiments, it should be appreciated that in certain embodiments, the FDR multiplicity adjusted p-value may be adjusted for at least one of age and gender, while in yet other embodiments, the FDR multiplicity adjusted p-value may be adjusted for both age and gender. On the other hand, where a test kit, detection device or apparatus, array, chip or panel is stratified for use with a single gender, it is also contemplated that in a test kit, detection device or apparatus, array, chip or panel at least 50%, or at least 55%, or at least 60%, or at least 65%, or at least 70%, or at last 75%, or at least 80%, or at least 85%, or at least 90%, or at least 95%, or all of the plurality of distinct food preparations, when adjusted for a single gender, have an average discriminatory p-value of ≤0.07 as determined by raw p-value or an average discriminatory p-value of ≤0.10 as determined by FDR multiplicity adjusted p-value. Furthermore, it should be appreciated that other stratifications (e.g., dietary preference, ethnicity, place of residence, genetic predisposition or family history, etc.) are also contemplated, and the person of ordinary skill in the art (PHOSITA) will be readily appraised of the appropriate choice of stratification.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate certain embodiments of the disclosure and does not pose a limitation on the scope of the any embodiments of the disclosure otherwise claimed. No language in the specification should be construed as indicating any non-claimed element essential to the practice of the disclosure.

Of course, it should be noted that the particular format of the test kit, detection device or apparatus, array, chip, or panel may vary considerably and contemplated formats include micro well plates, dip sticks, membrane-bound arrays, etc. Consequently, the solid carrier to which the food preparations are coupled may include wells of a multiwell plate, a (e.g., color-coded or magnetic) bead, or an adsorptive film (e.g., nitrocellulose or micro/nanoporous polymeric film), or an electrical sensor, (e.g., a printed copper sensor or microchip).

Consequently, the inventors also contemplate a method of identifying food items that trigger and/or exacerbate ADD/ADHD symptomology in patients that are diagnosed with or suspected to have ADD/ADHD. Most typically, such methods will include a step of contacting a food preparation with a bodily fluid (e.g., including, but not limited to, whole blood, plasma, serum, saliva, urine, or a fecal suspension) of a patient that is diagnosed with or suspected to have ADD/ADHD, and wherein the bodily fluid is associated with a gender identification. As noted before, the step of contacting is performed, in certain embodiments, under conditions that allow an immunoglobulin, e.g., IgG (or IgE or IgA or IgM or IgD) from the bodily fluid to bind to at least one component of the food preparation, and the IgG bound to the component(s) of the food preparation are then quantified/measured to obtain a signal. In some embodiments, the signal is then compared against a gender-stratified reference value (e.g., at least a 90th percentile value) for the food preparation using the gender identification to obtain a result, which is then used to update or generate a report (e.g., written medical report; oral report of results from doctor to patient; written or oral directive from physician based on results).

In certain embodiments, such methods will not be limited to a single food preparation, but will employ multiple different food preparations (i.e., a plurality of distinct food preparations). As noted before, suitable food preparations can be identified using various methods as described herein. In certain embodiments, the food preparations include foods 1-37, of Table 2, and/or items of Table 1. As also noted herein, in some embodiments, at least some, or all of the different food preparations have an average discriminatory p-value of ≤0.07 (or ≤0.065, or ≤0.06, or ≤0.055, or ≤0.05, or ≤0.045, or ≤0.04, or ≤0.035, or ≤0.03, or ≤0.025) as determined by raw p-value, and/or or an average discriminatory p-value of ≤0.10 (or ≤0.095, or ≤0.09, or ≤0.085, or ≤0.08, or ≤0.075, or ≤0.07) as determined by FDR multiplicity adjusted p-value.

While in certain embodiments food preparations are prepared from single food items as crude extracts, or crude filtered extracts, it is contemplated that food preparations can be prepared from mixtures of a plurality of food items (e.g., a mixture of citrus comprising lemon, orange, and a grapefruit, a mixture of yeast comprising baker's yeast and brewer's yeast, a mixture of rice comprising a brown rice and white rice, a mixture of sugars comprising honey, malt, and cane sugar. In some embodiments, it is also contemplated that food preparations can be prepared from purified food antigens or recombinant food antigens.

As provided in certain embodiments, the food preparation is immobilized on a solid surface (typically in an addressable manner), accordingly, it is contemplated that the step of measuring the immunoglobulin (e.g., IgG or other type of antibody) bound to the component of the food preparation is performed via an ELISA test. Exemplary solid surfaces include, but are not limited to, wells in a multiwell plate, such that each food preparation may be isolated to a separate microwell. In certain embodiments, the food preparation will be coupled to, or immobilized on, the solid surface. In other embodiments, the food preparation(s) will be coupled to a molecular tag that allows for binding to human immunoglobulins (e.g., IgG) in solution. See, e.g., Example 3.

Viewed from a different perspective, the inventors also contemplate a method of generating a test for identifying food items that trigger and/or exacerbate ADD/ADHD in patients diagnosed with or suspected of having ADD/ADHD. Thus, in certain embodiments, the method is for identifying triggering food items (i.e., trigger foods) among already diagnosed or suspected ADD/ADHD patients. Such test will typically include a step of obtaining one or more test results (e.g., ELISA) for various distinct food preparations, wherein the test results are based on bodily fluids (e.g., including, but not limited to, whole blood, plasma, serum, saliva, urine, fecal suspension) of patients diagnosed with or suspected to have ADD/ADHD and bodily fluids of a control group not diagnosed with or not suspected to have ADD/ADHD. In other embodiments, the method is for identifying triggering food items (i.e., trigger foods) among patients only suspected of having ADD/ADHD. In certain embodiments, the test results are then stratified by gender for each of the distinct food preparations, a different cutoff value for male and female patients for each of the distinct food preparations (e.g., cutoff value for male and female patients has a difference of at least 10% (abs)) is assigned for a predetermined percentile rank (e.g., 90th or 95th percentile).

As noted herein, and while not limiting to the inventive subject matter, it is contemplated that in certain embodiments, the plurality of distinct food preparations include at least two (or six, or ten, or 15) food preparations prepared from food items selected from the group consisting of foods 1-37 of Table 2, and/or items of Table 1. In other embodiments, the plurality of distinct food preparations are selected from the group consisting of at least 1 food preparation from foods 1-37 of Table 2, at least 2 food preparations from foods 1-37 of Table 2, at least 3 food preparations from foods 1-37 of Table 2, at least 4 food preparations from foods 1-37 of Table 2, at least 5 food preparations from foods 1-37 of Table 2, at least 6 food preparations from foods 1-37 of Table 2, at least 7 food preparations from foods 1-37 of Table 2, at least 8 food preparations from foods 1-37 of Table 2, at least 9 food preparations from foods 1-37 of Table 2, at least 10 food preparations from foods 1-37 of Table 2, at least 11 food preparations from foods 1-37 of Table 2, at least 12 food preparations from foods 1-37 of Table 2, at least 13 food preparations from foods 1-37 of Table 2, at least 14 food preparations from foods 1-37 of Table 2, at least 15 food preparations from foods 1-37 of Table 2, at least 16 food preparations from foods 1-37 of Table 2, at least 17 food preparations from foods 1-37 of Table 2, at least 18 food preparations from foods 1-37 of Table 2, at least 19 food preparations from foods 1-37 of Table 2, at least 20 food preparations from foods 1-37 of Table 2, at least 21 food preparations from foods 1-37 of Table 2, at least 22 food preparations from foods 1-37 of Table 2, at least 23 food preparations from foods 1-37 of Table 2, at least 24 food preparations from foods 1-37 of Table 2, at least 25 food preparations from foods 1-37 of Table 2, at least 26 food preparations from foods 1-37 of Table 2, at least 27 food preparations from foods 1-37 of Table 2, at least 28 food preparations from foods 1-37 of Table 2, at least 29 food preparations from foods 1-37 of Table 2, at least 30 food preparations from foods 1-37 of Table 2, at least 31 food preparations from foods 1-37 of Table 2, at least 32 food preparations from foods 1-37 of Table 2, at least 33 food preparations from foods 1-37 of Table 2, at least 34 food preparations from foods 1-37 of Table 2, at least 35 food preparations from foods 1-37 of Table 2, at least 36 food preparations from foods 1-37 of Table 2, at least 37 food preparations from foods 1-37 of Table 2. On the other hand, where new food items are tested, it should be appreciated that the distinct food preparations include a food preparation prepared from a food items other than foods 1-37 of Table 2. Regardless of the particular choice of food items, in certain embodiments, the plurality of distinct food preparations have an average discriminatory p-value of ≤0.07 (or ≤0.05, or ≤0.025) as determined by raw p-value or an average discriminatory p-value of ≤0.10 (or ≤0.08, or ≤0.07) as determined by FDR multiplicity adjusted p-value. In other embodiments, the plurality of distinct food preparations have an average discriminatory p-value as determined by raw p-value selected from the group consisting of about ≤0.07, about ≤0.065, about ≤0.06, about ≤0.055, about ≤0.05, about ≤0.045, about ≤0.04, about ≤0.035, about ≤0.03, about ≤0.025, and about ≤0.02. In yet other embodiments, the plurality of distinct food preparations have an average discriminatory p-value as determined by FDR multiplicity adjusted p-value selected from the group consisting of about ≤0.10, about ≤0.095, about ≤0.09, about ≤0.085, about ≤0.08, about ≤0.075, about ≤0.07, about ≤0.065, about ≤0.06, about ≤0.055, and about ≤0.05. Exemplary aspects and protocols, and considerations are provided in the experimental description below.

Thus, it should be appreciated that by having a high-confidence test system as described herein, the rate of false-positive and false negatives can be significantly reduced, and especially where the test systems and methods are gender stratified or adjusted for gender differences as shown below. Such advantages have heretofore not been realized and it is expected that the systems and methods presented herein will substantially increase the predictive power of food sensitivity tests for patients diagnosed with or suspected of having ADD/ADHD.

### EXAMPLES

### Example 1: General Protocol for Food Preparation Generation

Commercially available food extracts (available from Biomerica Inc., 17571 Von Karman Ave, Irvine, Calif. 92614) prepared from the edible portion of the respective raw foods were used to prepare ELISA plates following the manufacturer's instructions.

For some food extracts, the inventors expect that food extracts prepared with certain specific procedures to generate the food extracts, provides more superior results in detecting elevated immunoglobulin (e.g., IgG) reactivity in ADD/ADHD patients compared to commercially available food extracts. For example, in certain embodiments related to grains and nuts, a three-step procedure of generating food extracts may be used. The first step is a defatting step. In this step, lipids from grains and nuts are extracted by contacting the flour of grains and nuts with a non-polar solvent and collecting residue. Then, the defatted grain or nut flour are extracted by contacting the flour with elevated pH to obtain a mixture and removing the solid from the mixture to obtain the liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For another example, in certain embodiments related to meats and fish, a two-step procedure of generating food extracts may be used. The first step is an extraction step. In this step, extracts from raw, uncooked meats or fish are generated by emulsifying the raw, uncooked meats or fish in an aqueous buffer formulation in a high impact pressure processor. Then, solid materials are removed to obtain liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For still another example, in certain embodiments related to fruits and vegetables, a two-step procedure of generating food extracts may be used. The first step is an extraction step. In this step, liquid extracts from fruits or vegetables are generated using an extractor (e.g., masticating juicer, etc.) to pulverize foods and extract juice. Then, solid materials are removed to obtain liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

### Example 2: Blocking of ELISA Plates

To optimize signal to noise, plates will be blocked with a blocking buffer. In one embodiment, the blocking buffer includes 20-50 mM of a phosphate buffer (pH 4-9), bovine serum albumin (BSA) and a polyvinyl alcohol (PVA).

### Example 3: ELISA Preparation and Sample Testing

Food antigen preparations (i.e., food preparations) were immobilized onto respective microtiter wells following the manufacturer's instructions. For the assays, the food antigens (i.e., food preparations) were allowed to react with antibodies present in the patients' serum (e.g., IgG), and excess serum proteins were removed by a wash step. For detection of IgG antibody binding, enzyme labeled anti-IgG antibody conjugate was allowed to react with antigen-antibody complex. A color was developed by the addition of a substrate that reacts with the coupled enzyme. The color intensity was measured and is directly proportional to the concentration of IgG antibody specific to a particular food antigen.

### Example 4: Methodology to Determine Ranked Food List in Order of Ability of ELISA Signals to Distinguish ADD/ADHD from Control Subjects

Out of an initial selection (e.g., 100 food items, or 150 food items, or even more), samples can be eliminated prior to analysis due to low consumption in an intended, or target population. In addition, specific food items can be used as being representative of the larger more generic food group, especially where prior testing has established a correlation among different species within a generic group (e.g. in both genders, but also suitable for correlation for a single gender). For example, in one embodiment, green pepper could be dropped in favor of chili pepper as representative of the “pepper” food group, or sweet potato could be dropped in favor of potato as representative of the “potato” food group. In other embodiments, the final list foods will be less than 50 food items. In another embodiment, the final list of foods will be equal to or less than of 40 food items. In other embodiments, the final list of foods is selected from the group consisting of less than 50 food items, less than 49 food items, less than 48 food items, less than 47 food items, less than 46 food items, less than 45 food items, less than 44 food items, less than 43 food items, less than 42 food items, less than 41 food items, less than 40 food items, less than 39 food items, less than 38 food items, less than 37 food items, less than 36 food items, less than 35 food items, less than 34 food items, less than 33 food items, less than 32 food items, less than 31 food items, less than 30 food items, less than 29 food items, less than 28 food items, less than 27 food items, less than 26 food items, less than 25 food items, less than 24 food items, less than 23 food items, less than 22 food items, less than 21 food items, less than 20 food items less than 19 food items, less than 18 food items, less than 17 food items, less than 16 food items, less than 15 food items, less than 14 food items, less than 13 food items, less than 12 food items, less than 11 food items, less than 10 food items, less than 9 food items, less than 8 food items, less than 7 food items, less than 6 food items, less than 5 food items, less than 4 food items, less than 3 food items, and less than 2 food items.

Since the foods ultimately selected for the food panel will not be specific for a particular gender, a gender-neutral food list is necessary. Since the observed sample will be at least initially imbalanced by gender (e.g., Controls: 38.6% female, ADD/ADHD: 67.1% female), differences in ELISA signal magnitude strictly due to gender will be removed by modeling signal scores against gender using a two-sample t-test and storing the residuals for further analysis. For each of the tested foods (i.e., food preparations), residual signal scores will be compared between ADD/ADHD and controls using a permutation test on a two-sample t-test with a relative high number of resamplings (e.g., in certain embodiments >1,000 resamplings; in other embodiments >10,000 resamplings; in yet other embodiments >50,000 resamplings). The Satterthwaite approximation can then be used for the denominator degrees of freedom to account for lack of homogeneity of variances, and the 2-tailed permuted p-value will represent the raw p-value for each food. False Discovery Rates (FDR) among the comparisons, will be adjusted by any acceptable statistical procedures (e.g., Benjamin-Hochberg, Family-wise Error Rate (FWER), Per Comparison Error Rate (PCER), etc.).

Foods (i.e., food preparations) were then ranked according to their 2-tailed FDR multiplicity-adjusted p-values. Foods with adjusted p-values equal to or lower than the desired FDR threshold are deemed to have significantly higher signal scores among ADD/ADHD than control subjects and therefore deemed candidates for inclusion into a food panel. A typical result that is representative of the outcome of the statistical procedure is provided in Table 2. Here the ranking of foods is according to 2-tailed permutation T-test p-values with FDR adjustment.

Based on earlier experiments, the inventors contemplate that even for the same food preparation tested, the ELISA score for at least several food items (i.e., food preparations) will vary dramatically, and exemplary raw data are provided in Table 3. As should be readily appreciated, data unstratified by gender will therefore lose significant explanatory power where the same cutoff value is applied to raw data for male and female data. To overcome such disadvantage, the inventors therefore contemplate stratification of the data by gender as described below.

### Example 5: Statistical Method for Cutpoint Selection for Each Food

The determination of what ELISA signal scores would constitute a “positive” response can be made by summarizing the distribution of signal scores among the Control subjects. For each food (i.e., food preparations), ADD/ADHD subjects who have observed scores greater than or equal to selected quantiles of the Control subject distribution will be deemed “positive”. To attenuate the influence of any one subject on cutpoint determination, each food-specific and gender-specific dataset will be bootstrap resampled 1000 times. Within each bootstrap replicate, the 90th and 95th percentiles of the Control signal scores will be determined. Each ADD/ADHD subject in the bootstrap sample will be compared to the 90th and 95% percentiles to determine whether he/she had a “positive” response. The final 90th and 95th percentile-based cutpoints for each food and gender will be computed as the average 90th and 95th percentiles across the 1000 samples. The number of foods for which each ADD/ADHD subject will be rated as “positive” was computed by pooling data across foods. Using such method, the inventors will be now able to identify cutoff values for a predetermined percentile rank that in most cases was substantially different as can be taken from Table 4.

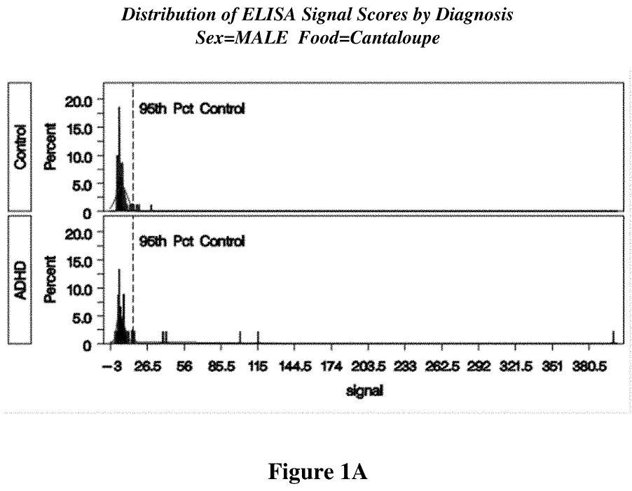

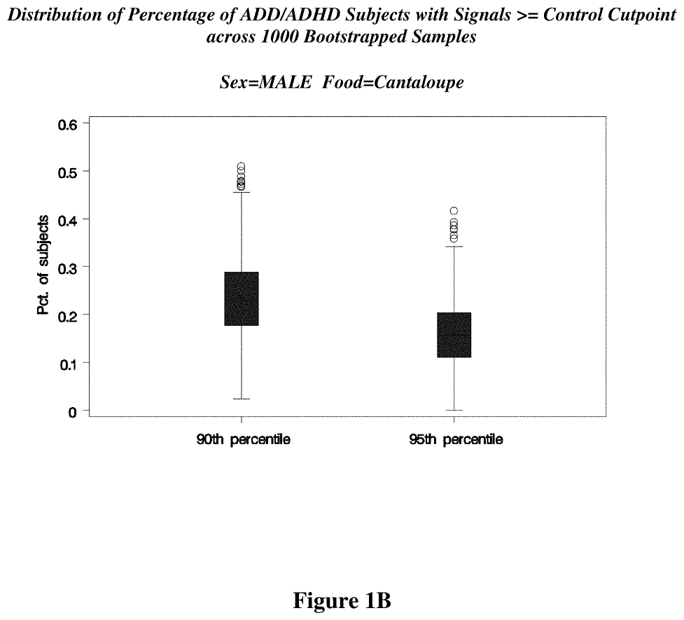

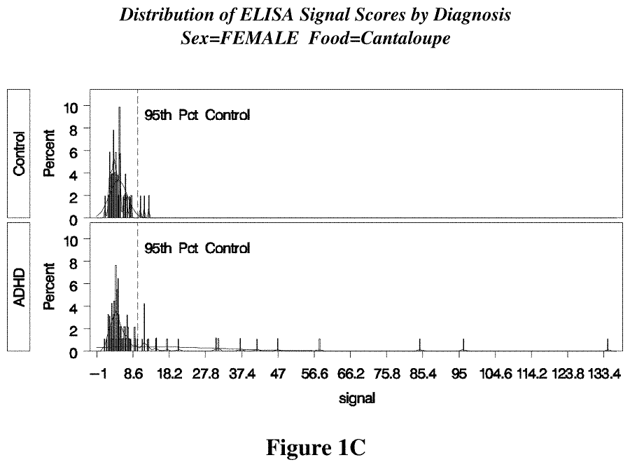

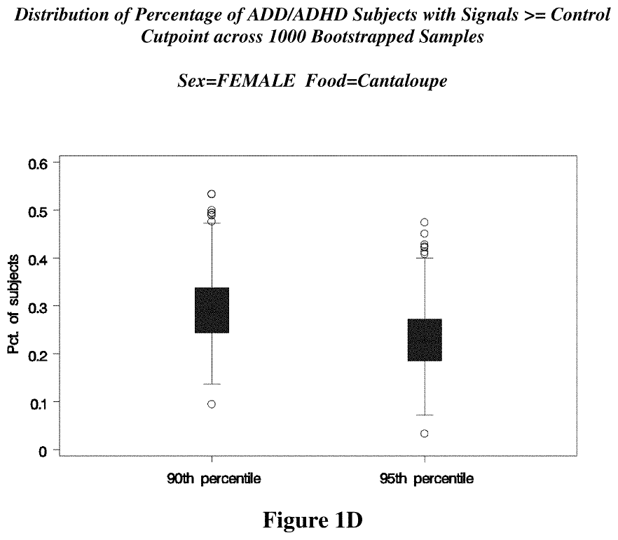

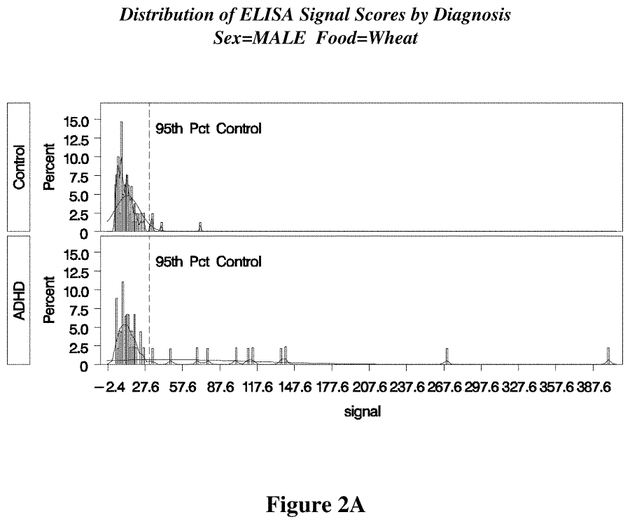

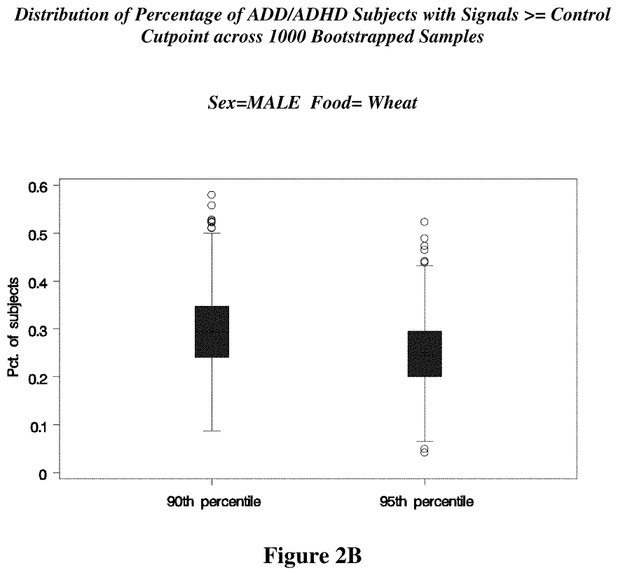

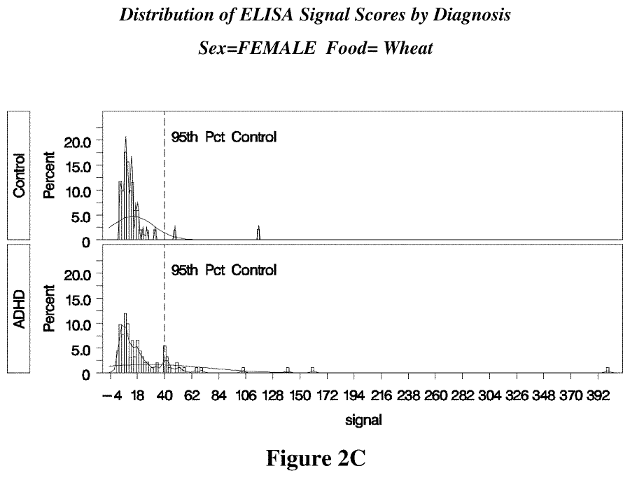

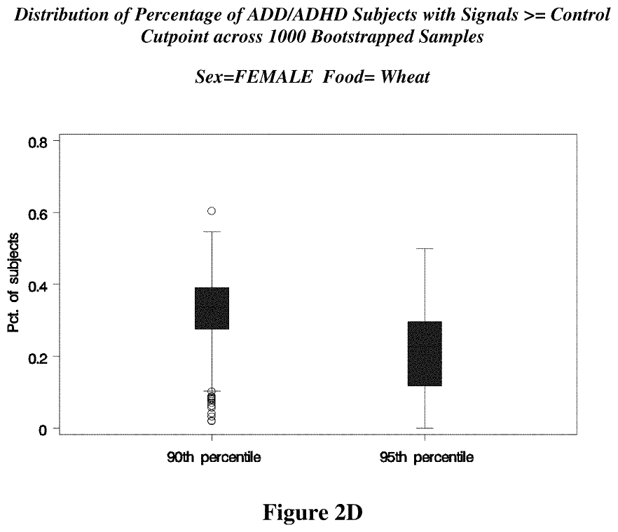

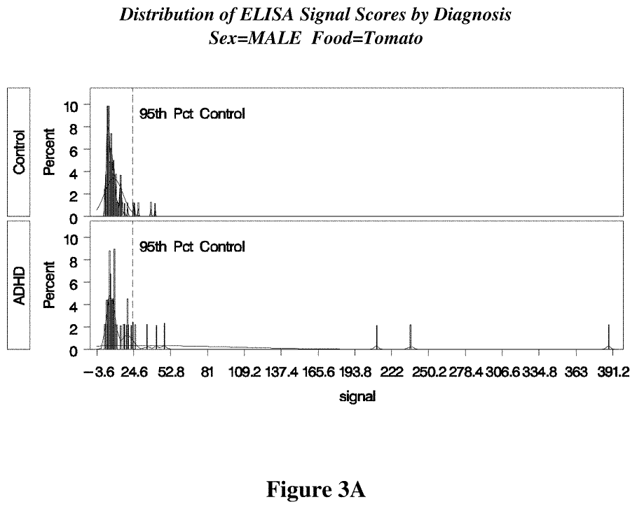

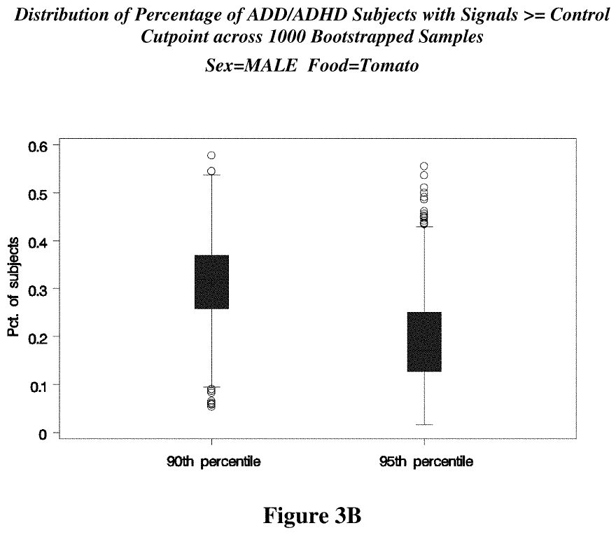

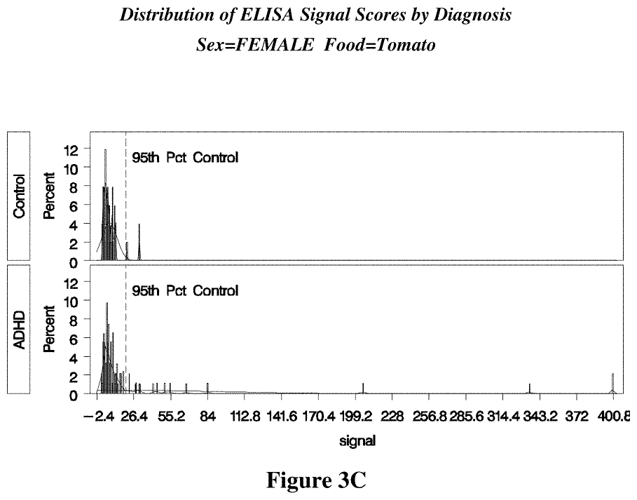

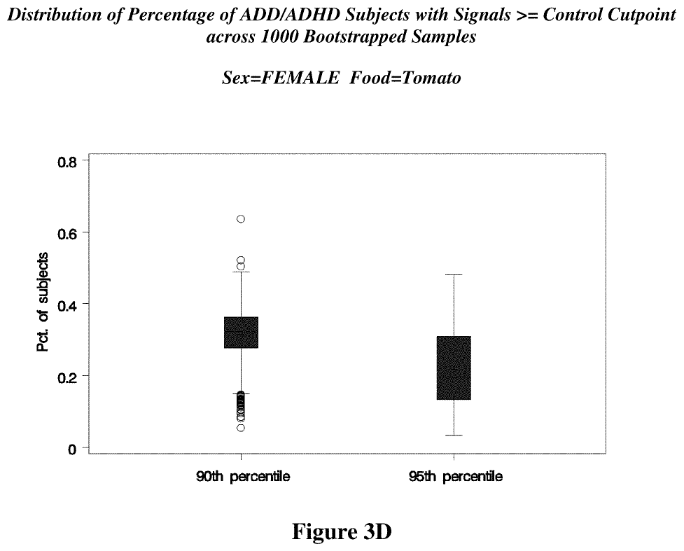

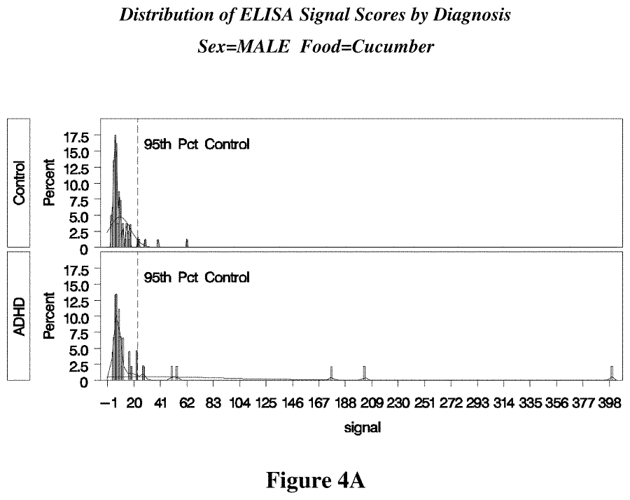

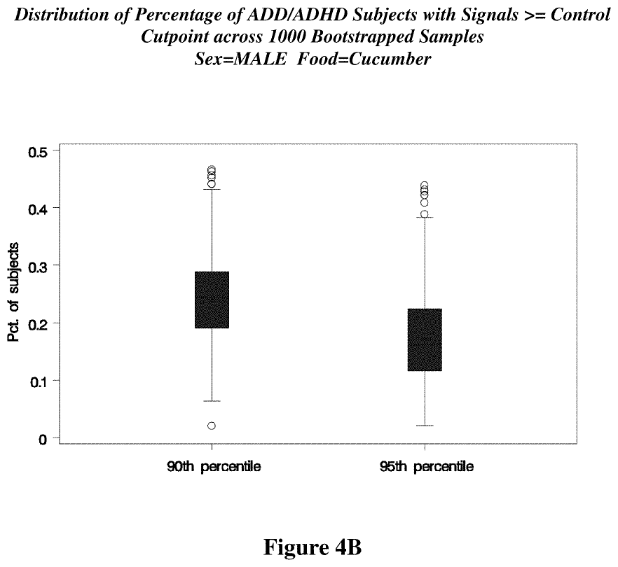

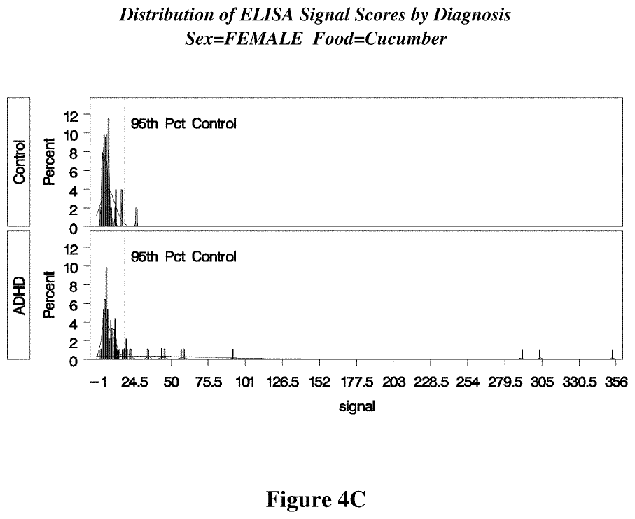

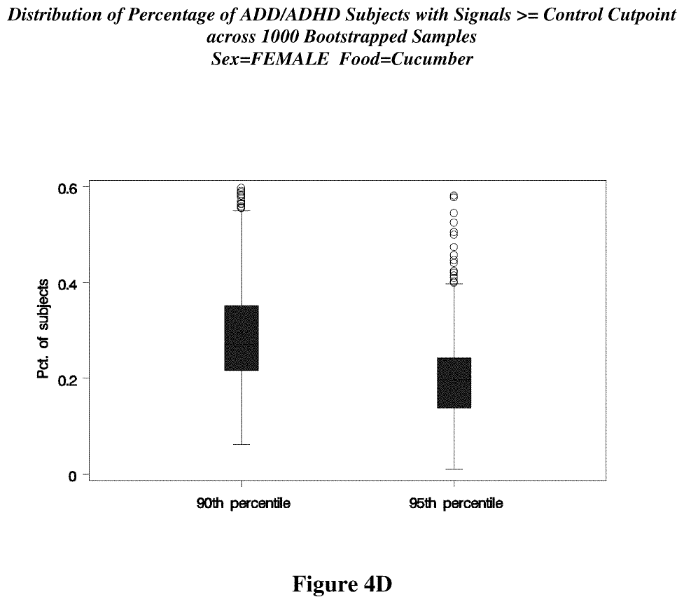

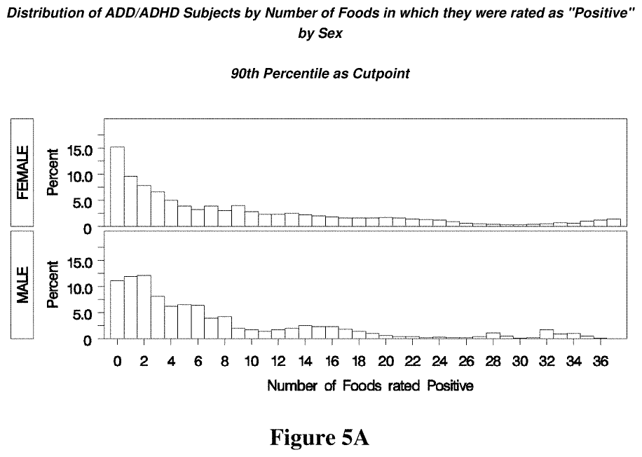

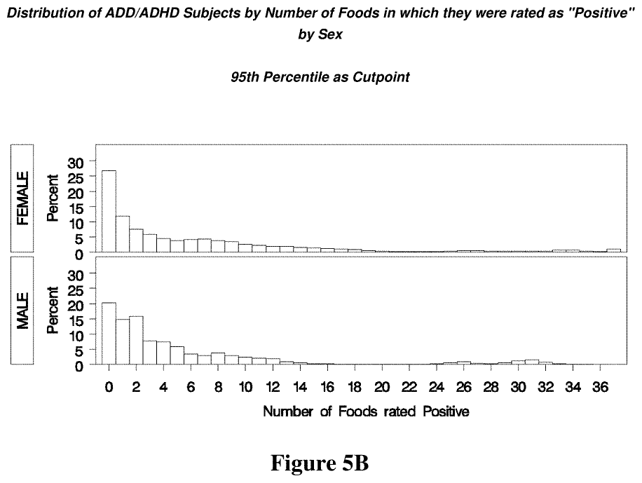

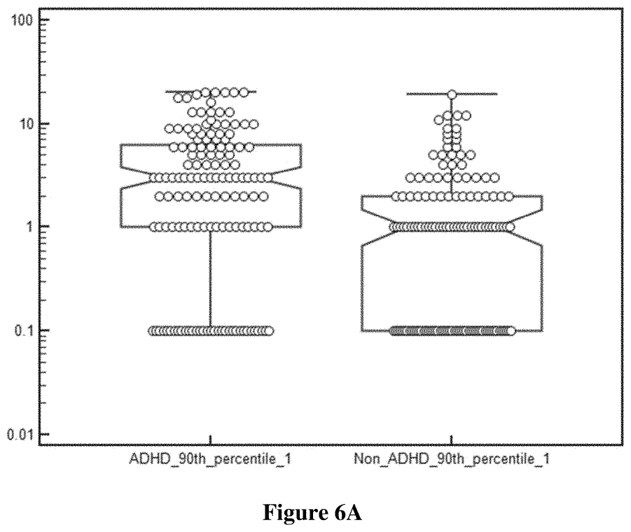

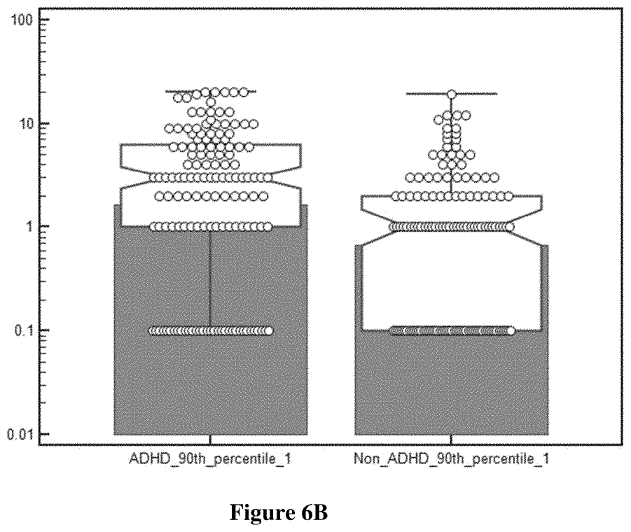

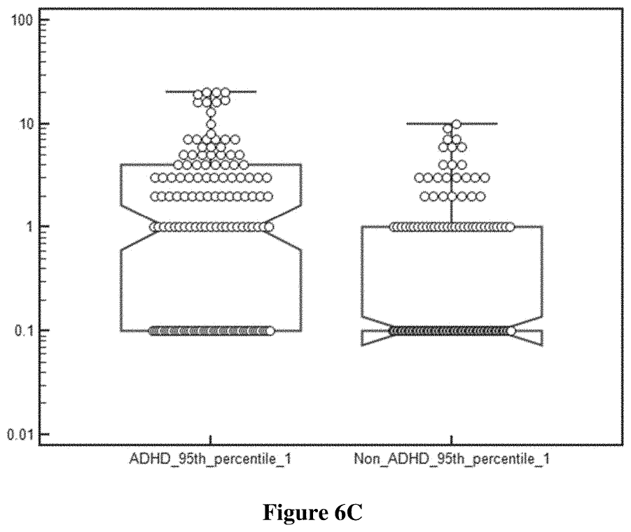

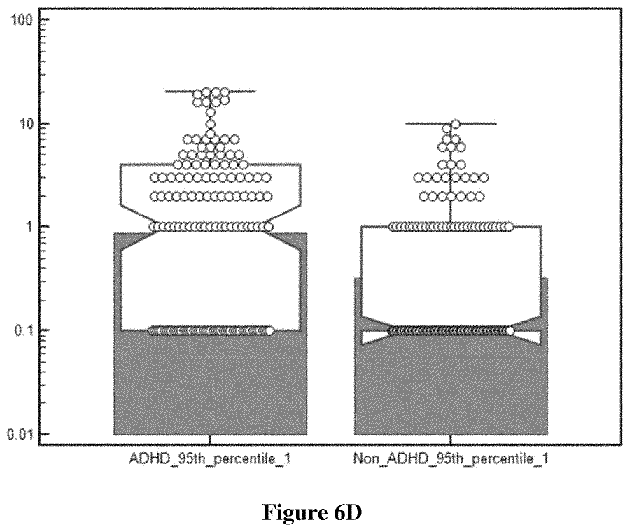

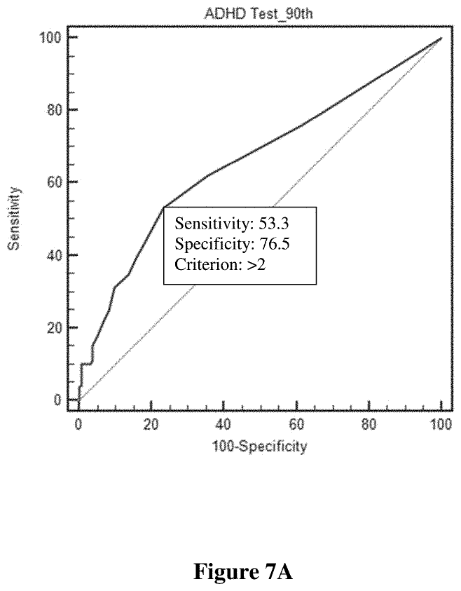

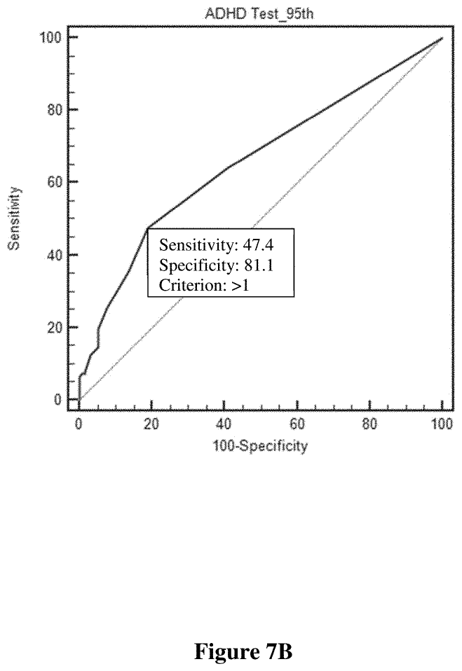

Typical examples for the gender difference in IgG response in blood with respect to cantaloupe is shown in FIGS. 1A-1D, where FIG. 1A shows the signal distribution in men along with the 95^{th} percentile cutoff as determined from the male control population. FIG. 1B shows the distribution of percentage of male ADD/ADHD subjects exceeding the 90^{th} and 95^{th} percentile, while FIG. 1C shows the signal distribution in women along with the 95^{th} percentile cutoff as determined from the female control population. FIG. 1D shows the distribution of percentage of female ADD/ADHD subjects exceeding the 90^{th} and 95^{th} percentile. In the same fashion, FIGS. 2A-2D exemplarily depict the differential response to wheat, FIGS. 3A-3D exemplarily depict the differential response to tomato, and FIGS. 4A-4D exemplarily depict the differential response to cucumber. FIGS. 5A-5B show the distribution of ADD/ADHD subjects by number of foods that were identified as trigger foods at the 90^{th} percentile (5A) and 95^{th} percentile (5B). Inventors contemplate that regardless of the particular food items (i.e., food preparations), male and female responses will be notably distinct.

It should be noted that nothing in the art have provided any predictable food groups related to ADD/ADHD that is gender-stratified. Thus, a discovery of food items (i.e., food preparations) that show distinct responses by gender is a surprising result, which could not be obviously expected in view of all previously available arts. In other words, selection of food items based on gender stratification provides an unexpected technical effect such that statistical significances for particular food items as triggering food among male or female ADD/ADHD patients have been significantly improved.

### Example 6: Normalization of IgG Response Data

While the raw data of the patient's IgG response results can be used to compare strength of response among given foods, it is also contemplated that the IgG response results of a patient are normalized and indexed to generate unit-less numbers for comparison of relative strength of response to a given food. For example, one or more of a patient's food specific IgG results (e.g., IgG specific to orange and IgG specific to malt) can be normalized to the patient's total IgG. The normalized value of the patient's IgG specific to orange can be 0.1 and the normalized value of the patient's IgG specific to malt can be 0.3. In this scenario, the relative strength of the patient's response to malt is three times higher compared to orange. Then, the patient's sensitivity to malt and orange can be indexed as such.

In other examples, one or more of a patient's food specific IgG results (e.g., IgG specific to shrimp and IgG specific to pork) can be normalized to the global mean of that patient's food specific IgG results. The global means of the patient's food specific IgG can be measured by total amount of the patient's food specific IgG. In this scenario, the patient's specific IgG to shrimp can be normalized to the mean of patient's total food specific IgG (e.g., mean of IgG levels to shrimp, pork, Dungeness crab, chicken, peas, etc.). However, it is also contemplated that the global means of the patient's food specific IgG can be measured by the patient's IgG levels to a specific type of food via multiple tests. If the patient has been tested for his sensitivity to shrimp five times and to pork seven times previously, the patient's new IgG values to shrimp or to pork are normalized to the mean of five-times test results to shrimp or the mean of seven-times test results to pork. The normalized value of the patient's IgG specific to shrimp can be 6.0 and the normalized value of the patient's IgG specific to pork can be 1.0. In this scenario, the patient has six times higher sensitivity to shrimp at this time compared to his average sensitivity to shrimp, but substantially similar sensitivity to pork. Then, the patient's sensitivity to shrimp and pork can be indexed based on such comparison.

### Example 7: Methodology to Determine the Subset of ADD/ADHD Patients with Food Sensitivities that Underlie ADD/ADHD

While it is suspected that food sensitivities plays a substantial role in signs and symptoms of ADD/ADHD, some ADD/ADHD patients may not have food sensitivities that underlie ADD/ADHD. Those patients would not be benefit from dietary intervention to treat signs and symptoms of ADD/ADHD. To determine the subset of such patients, body fluid samples of ADD/ADHD patients and non-ADD/ADHD patients can be tested with ELISA test using test devices with up to 37 food samples.

Table 5A and Table 5B provide exemplary raw data. As should be readily appreciated, the data indicate number of positive results out of 37 sample foods based on 90^{th} percentile value (Table 5A) or 95^{th} percentile value (Table 5B). The first column is ADD/ADHD (n=137); second column is non-ADD/ADHD (n=132) by ICD-10 code. Average and median number of positive foods was computed for ADD/ADHD and non-ADD/ADHD patients. From the raw data shown in Table 5A and Table 5B, average and standard deviation of the number of positive foods (i.e., trigger foods) was computed for ADD/ADHD and non-ADD/ADHD patients. Additionally, the number and percentage of patients with zero positive foods was calculated for both ADD/ADHD and non-ADD/ADHD. The number and percentage of patients with zero positive foods in the ADD/ADHD population is approximately 40% lower than the percentage of patients with zero positive foods in the non-ADD/ADHD population (24.1% vs. 38.6%, respectively) based on 90^{th} percentile value (Table 5A), and the percentage of patients in the ADD/ADHD population with zero positive foods is also significantly lower (i.e. approximately 40% lower) than that seen in the non-ADD/ADHD population (35.8% vs. 59.1%, respectively) based on 95^{th} percentile value (Table 5B). Thus, it can be easily appreciated that the ADD/ADHD patient having sensitivity to zero positive foods is unlikely to have food sensitivities underlying their signs and symptoms of ADD/ADHD.

Table 6A and Table 7A show exemplary statistical data summarizing the raw data of two patient populations shown in Table 5A. The statistical data includes normality, arithmetic mean, median, percentiles and 95% confidence interval (CI) for the mean and median representing number of positive foods (i.e., trigger foods) in the ADD/ADHD population and the non-ADD/ADHD population. Table 6B and Table 7B show exemplary statistical data summarizing the raw data of two patient populations shown in Table 5B. The statistical data includes normality, arithmetic mean, median, percentiles and 95% confidence interval (CI) for the mean and median representing number of positive foods in the ADD/ADHD population and the non-ADD/ADHD population.

Table 8A and Table 9A show exemplary statistical data summarizing the raw data of two patient populations shown in Table 5A. In Tables 8A and 9A, the raw data was transformed by logarithmic transformation to improve the data interpretation. Table 8B and Table 9B show another exemplary statistical data summarizing the raw data of two patient populations shown in Table 5B. In Tables 8B and 9B, the raw data was transformed by logarithmic transformation to improve the data interpretation.

Table 10A and Table 11A show exemplary statistical data of an independent T-test (Table 10A, logarithmically transformed data) and a Mann-Whitney test (Table 11A) to compare the geometric mean number of positive foods (i.e., trigger foods) between the ADD/ADHD and non-ADD/ADHD samples. The data shown in Table 10A and Table 11A indicate statistically significant differences in the geometric mean of positive number of foods between the ADD/ADHD population and the non-ADD/ADHD population. In both statistical tests, it is shown that the number of positive responses with 37 food samples is significantly higher in the ADD/ADHD population than in the non-ADD/ADHD population with an average discriminatory p-value of ≤0.0001. These statistical data is also illustrated as a box and whisker plot in FIG. 6A, and a notched box and whisker plot in FIG. 6B.

Table 10B and Table 11B show exemplary statistical data of an independent T-test (Table 10A, logarithmically transformed data) and a Mann-Whitney test (Table 11B) to compare the geometric mean number of positive foods (i.e., trigger foods) between the ADD/ADHD and non-ADD/ADHD samples. The data shown in Table 10B and Table 11B indicate statistically significant differences in the geometric mean of positive number of foods between the ADD/ADHD population and the non-ADD/ADHD population. In both statistical tests, it is shown that the number of positive responses with 37 food samples is significantly higher in the ADD/ADHD population than in the non-ADD/ADHD population with an average discriminatory p-value of ≤0.0001. These statistical data is also illustrated as a box and whisker plot in FIG. 6C, and a notched box and whisker plot in FIG. 6D.

Table 12A shows exemplary statistical data of a Receiver Operating Characteristic (ROC) curve analysis of data shown in Tables 5A-11A to determine the diagnostic power of the test used in Table 5 at discriminating ADD/ADHD from non-ADD/ADHD subjects. When a cutoff criterion of more than 2 positive foods (i.e., trigger foods) is used, the test yields a data with 53.3% sensitivity and 76.5% specificity, with an area under the curve (AUROC) of 0.660. The p-value for the ROC is significant at a p-value of <0.0001. FIG. 7A illustrates the ROC curve corresponding to the statistical data shown in Table 12A. Because the statistical difference between the ADD/ADHD population and the non-ADD/ADHD population is significant when the test results are cut off to a positive number of 2, the number of foods for which a patient tests positive could be used as a confirmation of the primary clinical diagnosis of ADD/ADHD, and whether it is likely that food sensitivities underlies on the patient's signs and symptoms of ADD/ADHD. Therefore, the above test can be used as another ‘rule in’ test to add to currently available clinical criteria for diagnosis for ADD/ADHD.

As shown in Tables 5A-12A, and FIG. 7A, based on 90^{th} percentile data, the number of positive foods (i.e., trigger foods) seen in ADD/ADHD vs. non-ADD/ADHD subjects is significantly different whether the geometric mean or median of the data is compared. The number of positive foods that a person has is indicative of the presence of ADD/ADHD in subjects. The test has discriminatory power to detect ADD/ADHD with 53.3% sensitivity and 76.5% specificity. Additionally, the absolute number and percentage of subjects with 0 positive foods is also very different in ADD/ADHD vs. non-ADD/ADHD subjects, with a far lower percentage of ADD/ADHD subjects (24.1%) having 0 positive foods than non-ADD/ADHD subjects (38.6%). The data suggests a subset of ADD/ADHD patients may have ADD/ADHD due to other factors than diet, and may not benefit from dietary restriction.

Table 12B shows exemplary statistical data of a Receiver Operating Characteristic (ROC) curve analysis of data shown in Tables 5B-11B to determine the diagnostic power of the test used in Table 5 at discriminating ADD/ADHD from non-ADD/ADHD subjects. When a cutoff criterion of more than 1 positive foods (i.e., trigger foods) is used, the test yields a data with 47.4% sensitivity and 81.1% specificity, with an area under the curve (AUROC) of 0.659. The p-value for the ROC is significant at a p-value of <0.0001. FIG. 7B illustrates the ROC curve corresponding to the statistical data shown in Table 12B. Because the statistical difference between the ADD/ADHD population and the non-ADD/ADHD population is significant when the test results are cut off to positive number of >1, the number of foods that a patient tests positive could be used as a confirmation of the primary clinical diagnosis of ADD/ADHD, and whether it is likely that food sensitivities underlies on the patient's signs and symptoms of ADD/ADHD. Therefore, the above test can be used as another ‘rule in’ test to add to currently available clinical criteria for diagnosis for ADD/ADHD.

As shown in Tables 5B-12B, and FIG. 7B, based on 95^{th} percentile data, the number of positive foods (i.e., trigger foods) seen in ADD/ADHD vs. non-ADD/ADHD subjects is significantly different whether the geometric mean or median of the data is compared. The number of positive foods that a person has is indicative of the presence of ADD/ADHD in subjects. The test has discriminatory power to detect ADD/ADHD with 47.4% sensitivity and 81.1% specificity. Additionally, the absolute number and percentage of subjects with 0 positive foods is also very different in ADD/ADHD vs. non-ADD/ADHD subjects, with a far lower percentage of ADD/ADHD subjects (35.8%) having 0 positive foods than non-ADD/ADHD subjects (59.1%). The data suggests a subset of ADD/ADHD patients may have ADD/ADHD due to other factors than diet, and may not benefit from dietary restriction.

### Example 8: Method for Determining Distribution of Per-Person Number of Foods Declared “Positive”

To determine the distribution of number of “positive” foods (i.e., trigger foods) per person and measure the diagnostic performance, the analysis will be performed with 37 food items from Table 2, which shows most positive responses to ADD/ADHD patients. To attenuate the influence of any one subject on this analysis, each food-specific and gender-specific dataset will be bootstrap resampled 1000 times. Then, for each food item in the bootstrap sample, sex-specific cutpoint will be determined using the 90th and 95th percentiles of the control population. Once the sex-specific cutpoints are determined, the sex-specific cutpoints will be compared with the observed ELISA signal scores for both control and ADD/ADHD subjects. In this comparison, if the observed signal is equal or more than the cutpoint value, then it will be determined “positive” food, and if the observed signal is less than the cutpoint value, then it will be determined “negative” food.

Once all food items (i.e., food preparations) were determined either positive or negative, the results of the 74 (37 foods×2 cutpoints) calls for each subject will be saved within each bootstrap replicate. Then, for each subject, 37 calls will be summed using 90^{th} percentile as cutpoint to get “Number of Positive Foods (90^{th}),” and the rest of 37 calls will be summed using 95^{th} percentile to get “Number of Positive Foods (95^{th}).”) Then, within each replicate, “Number of Positive Foods (90^{th})” and “Number of Positive Foods (95^{th})” will be summarized across subjects to get descriptive statistics for each replicate as follows: 1) overall means equals to the mean of means, 2) overall standard deviation equals to the mean of standard deviations, 3) overall medial equals to the mean of medians, 4) overall minimum equals to the minimum of minimums, and 5) overall maximum equals to maximum of maximum. In this analysis, to avoid non-integer “Number of Positive Foods” when computing frequency distribution and histogram, the authors will pretend that the 1000 repetitions of the same original dataset were actually 999 sets of new subjects of the same size added to the original sample. Once the summarization of data is done, frequency distributions and histograms will be generated for both “Number of Positive Foods (90^{th})” and “Number of Positive Foods (95^{th})” for both genders and for both ADD/ADHD subjects and control subjects using programs “a_pos_foods.sas, a_pos_foods_by_dx.sas”.

### Example 9: Method for Measuring Diagnostic Performance

To measure diagnostic performance for each food items (i.e., food preparations) for each subject, we will use data of “Number of Positive Foods (90^{th})” and “Number of Positive Foods (95^{th})” for each subject within each bootstrap replicate described above. In this analysis, the cutpoint was set to 1. Thus, if a subject has one or more “Number of Positive Foods (90^{th})”, then the subject will be called “Has ADD/ADHD.” If a subject has less than one “Number of Positive Foods (90^{th})”, then the subject will be called “Does Not Have ADD/ADHD.” When all calls were made, the calls were compared with actual diagnosis to determine whether a call was a True Positive (TP), True Negative (TN), False Positive (FP), or False Negative (FN). The comparisons will be summarized across subjects to get the performance metrics of sensitivity, specificity, positive predictive value, and negative predictive value for both “Number of Positive Foods (90^{th})” and “Number of Positive Foods (95^{th})” when the cutpoint is set to 1 for each method. Each (sensitivity, 1-specificity) pair becomes a point on the ROC curve for this replicate.

To increase the accuracy, the analysis above will be repeated by incrementing cutpoint from 2 up to 37, and repeated for each of the 1000 bootstrap replicates. Then the performance metrics across the 1000 bootstrap replicates will be summarized by calculating averages using a program “t_pos_foods_by_dx.sas”. The results of diagnostic performance for female and male are shown in Tables 13A and 13B (90th percentile) and Tables 14 A and 14B (95th percentile).

Of course, it should be appreciated that certain variations in the food preparations may be made without altering the inventive subject matter presented herein. For example, where the food item was yellow onion, that item should be understood to also include other onion varieties that were demonstrated to have equivalent activity in the tests. Indeed, the inventors have noted that for each tested food preparation, certain other related food preparations also tested in the same or equivalent manner (data not shown). Thus, it should be appreciated that each tested and claimed food preparation will have equivalent related preparations with demonstrated equal or equivalent reactions in the test.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

TABLE 1

Abalone
Adlay
Almond
American Cheese
Apple
Artichoke
Asparagus
Avocado
Baby Bok Choy
Bamboo shoots
Banana
Barley, whole grain
Beef
Beets
Beta-lactoglobulin
Blueberry
Broccoli
Buckwheat
Butter
Cabbage
Cane sugar
Cantaloupe
Caraway
Carrot
Casein
Cashew
Cauliflower
Celery
Chard
Cheddar Cheese
Chick Peas
Chicken
Chili pepper
Chocolate
Cinnamon
Clam
Cocoa Bean
Coconut
Codfish
Coffee
Cola nut
Corn
Cottage cheese
Cow's milk
Crab
Cucumber
Cured Cheese
Cuttlefish
Duck
Durian
Eel
Egg White (separate)
Egg Yolk (separate)
Egg, white/yolk (comb.)
Eggplant
Garlic
Ginger
Gluten - Gliadin
Goat's milk
Grape, white/concord
Grapefruit
Grass Carp
Green Onion
Green pea
Green pepper
Guava
Hair Tail
Hake
Halibut
Hazelnut
Honey
Kelp
Kidney bean
Kiwi Fruit
Lamb
Leek
Lemon
Lentils
Lettuce, Iceberg
Lima bean
Lobster
Longan
Mackerel
Malt
Mango
Marjoram
Millet
Mung bean
Mushroom
Mustard seed
Oat
Olive
Onion
Orange
Oyster
Papaya
Paprika
Parsley
Peach
Peanut
Pear
Pepper, Black
Pineapple
Pinto bean
Plum
Pork
Potato
Rabbit
Rice
Roquefort Cheese
Rye
Saccharine
Safflower seed
Salmon
Sardine
Scallop
Sesame
Shark fin
Sheep's milk
Shrimp
Sole
Soybean
Spinach
Squashes
Squid
Strawberry
String bean
Sunflower seed
Sweet potato
Swiss cheese
Taro
Tea, black
Tobacco
Tomato
Trout
Tuna
Turkey
Vanilla
Walnut, black
Watermelon
Welch Onion
Wheat
Wheat bran
Yeast (S. cerevisiae)
Yogurt
FOOD ADDITIVES
Arabic Gum
Carboxymethyl Cellulose
Carrageneenan
FD&C Blue #1
FD&C Red #3
FD&C Red #40
FD&C Yellow #5
FD&C Yellow #6
Gelatin
Guar Gum
Maltodextrin
Pectin
Whey
Xanthan Gum

TABLE 2
Ranking of Foods according to 2-tailed Permutation
T-test p-values with FDR adjustment

| | | | FDR |
|---|---|---|---|
| | | Raw | Multiplicity-adj |
| Rank | Food | p-value | p-value |
| 1 | Cantaloupe | 0.0000 | 0.0017 |
| 2 | Wheat | 0.0000 | 0.0017 |
| 3 | Tomato | 0.0001 | 0.0029 |
| 4 | Cucumber | 0.0001 | 0.0030 |
| 5 | Squashes | 0.0003 | 0.0046 |
| 6 | Almond | 0.0003 | 0.0046 |
| 7 | Egg | 0.0004 | 0.0048 |
| 8 | Cauliflower | 0.0005 | 0.0048 |
| 9 | Pinto_Bean | 0.0005 | 0.0048 |
| 10 | Broccoli | 0.0006 | 0.0048 |
| 11 | Orange | 0.0008 | 0.0063 |
| 12 | Butter | 0.0011 | 0.0077 |
| 13 | Corn | 0.0012 | 0.0077 |
| 14 | Lettuce | 0.0013 | 0.0081 |
| 15 | Rye | 0.0016 | 0.0092 |
| 16 | Peach | 0.0026 | 0.0139 |
| 17 | Green_Pea | 0.0031 | 0.0156 |
| 18 | Carrot | 0.0035 | 0.0168 |
| 19 | Tea | 0.0051 | 0.0233 |
| 20 | Mustard | 0.0058 | 0.0249 |
| 21 | Strawberry | 0.0062 | 0.0253 |
| 22 | Celery | 0.0093 | 0.0351 |
| 23 | Green_Pepper | 0.0094 | 0.0351 |
| 24 | Garlic | 0.0101 | 0.0358 |
| 25 | Oat | 0.0104 | 0.0358 |
| 26 | Onion | 0.0120 | 0.0396 |
| 27 | Banana | 0.0139 | 0.0443 |
| 28 | Eggplant | 0.0156 | 0.0479 |
| 29 | Cabbage | 0.0162 | 0.0480 |
| 30 | Safflower | 0.0168 | 0.0480 |
| 31 | Olive | 0.0175 | 0.0487 |
| 32 | Cottage_Ch_{—} | 0.0190 | 0.0512 |
| 33 | Grapefruit | 0.0208 | 0.0541 |
| 34 | Walnut_Blk | 0.0243 | 0.0615 |
| 35 | Cow_Milk | 0.0256 | 0.0629 |
| 36 | Soybean | 0.0300 | 0.0717 |
| 37 | Chili_Pepper | 0.0376 | 0.0874 |
| 38 | Tobacco | 0.0481 | 0.1088 |
| 39 | Malt | 0.0507 | 0.1118 |
| 40 | Cinnamon | 0.0545 | 0.1171 |
| 41 | Cane_Sugar | 0.0619 | 0.1299 |
| 42 | Potato | 0.0665 | 0.1362 |
| 43 | Millet | 0.0704 | 0.1407 |
| 44 | Sunflower_Sd | 0.0807 | 0.1577 |
| 45 | Crab | 0.0850 | 0.1625 |
| 46 | Lima_Bean | 0.1080 | 0.2020 |
| 47 | Chocolate | 0.1114 | 0.2038 |
| 48 | Spinach | 0.1205 | 0.2159 |
| 49 | Buck_Wheat | 0.1283 | 0.2241 |
| 50 | Honey | 0.1303 | 0.2241 |
| 51 | Pineapple | 0.1376 | 0.2274 |
| 52 | Yeast_Brewer | 0.1382 | 0.2274 |
| 53 | Rice | 0.1401 | 0.2274 |
| 54 | Apple | 0.1962 | 0.3125 |
| 55 | Mushroom | 0.2001 | 0.3128 |
| 56 | Yeast_Baker | 0.2551 | 0.3918 |
| 57 | Avocado | 0.2707 | 0.4075 |
| 58 | Cheddar_Ch_{—} | 0.2803 | 0.4075 |
| 59 | Sesame | 0.2821 | 0.4075 |
| 60 | Clam | 0.2843 | 0.4075 |
| 61 | Pork | 0.2982 | 0.4204 |
| 62 | Blueberry | 0.3534 | 0.4903 |
| 63 | Swiss_Ch_{—} | 0.3654 | 0.4987 |
| 64 | Barley | 0.3841 | 0.5162 |
| 65 | String_Bean | 0.4313 | 0.5707 |
| 66 | Peanut | 0.4626 | 0.6028 |
| 67 | Scallop | 0.4997 | 0.6414 |
| 68 | Lemon | 0.5191 | 0.6565 |
| 69 | Goat_Milk | 0.5378 | 0.6624 |
| 70 | Oyster | 0.5392 | 0.6624 |
| 71 | Lobster | 0.5621 | 0.6809 |
| 72 | Chicken | 0.6743 | 0.8055 |
| 73 | Amer_Cheese | 0.7096 | 0.8312 |
| 74 | Coffee | 0.7152 | 0.8312 |
| 75 | Beef | 0.7616 | 0.8733 |
| 76 | Codfish | 0.8330 | 0.9426 |
| 77 | Salmon | 0.8779 | 0.9646 |
| 78 | Cashew | 0.8950 | 0.9646 |
| 79 | Sardine | 0.8972 | 0.9646 |
| 80 | Parsley | 0.9073 | 0.9646 |
| 81 | Turkey | 0.9086 | 0.9646 |
| 82 | Grape | 0.9376 | 0.9780 |
| 83 | Yogurt | 0.9439 | 0.9780 |
| 84 | Cola_Nut | 0.9742 | 0.9931 |
| 85 | Sweet_Pot_{—} | 0.9840 | 0.9931 |
| 86 | Trout | 0.9931 | 0.9931 |

TABLE 3
Basic Descriptive Statistics of ELISA Score by
Food and Gender Comparing ADD/ADHD to Control

| | | | ELISA Score | | | | |
|---|---|---|---|---|---|---|---|
| Sex | Food | Diagnosis | N | Mean | SD | Min | Max |
| FEMALE | Almond | Control | 51 | 4.057 | 1.630 | 1.270 | 8.090 |
| | | ADHD | 92 | 6.654 | 7.307 | 0.700 | 49.320 |
| | | Diff (1-2) | — | −2.597 | 5.950 | — | — |
| | Amer_Cheese | Control | 51 | 32.961 | 66.654 | 0.100 | 400.00 |
| | | ADHD | 92 | 32.901 | 33.849 | 1.210 | 148.00 |
| | | Diff (1-2) | — | 0.060 | 48.113 | — | — |
| | Apple | Control | 51 | 4.792 | 6.600 | 0.100 | 44.160 |
| | | ADHD | 92 | 5.578 | 8.466 | 0.110 | 57.720 |
| | | Diff (1-2) | — | −0.785 | 7.855 | — | — |
| | Avocado | Control | 51 | 2.144 | 1.129 | 0.100 | 5.560 |
| | | ADHD | 92 | 3.177 | 4.055 | 0.100 | 33.660 |
| | | Diff (1-2) | — | −1.032 | 3.326 | — | — |
| | Banana | Control | 51 | 3.042 | 2.909 | 0.100 | 17.210 |
| | | ADHD | 92 | 6.176 | 11.816 | 0.100 | 105.04 |
| | | Diff (1-2) | — | −3.134 | 9.649 | — | — |
| | Barley | Control | 51 | 3.867 | 3.919 | 0.100 | 25.110 |
| | | ADHD | 92 | 9.954 | 19.858 | 0.100 | 133.34 |
| | | Diff (1-2) | — | −6.087 | 16.123 | — | — |
| | Beef | Control | 51 | 9.007 | 12.047 | 1.030 | 81.660 |
| | | ADHD | 92 | 12.865 | 40.258 | 1.270 | 380.80 |
| | | Diff (1-2) | — | −3.858 | 33.128 | — | — |
| | Blueberry | Control | 51 | 3.533 | 4.712 | 0.100 | 26.460 |
| | | ADHD | 92 | 4.293 | 5.077 | 0.860 | 34.650 |
| | | Diff (1-2) | — | −0.760 | 4.951 | — | — |
| | Broccoli | Control | 51 | 5.211 | 4.424 | 0.110 | 29.600 |
| | | ADHD | 92 | 9.398 | 10.582 | 2.570 | 91.250 |
| | | Diff (1-2) | — | −4.187 | 8.900 | — | — |
| | Buck_Wheat | Control | 51 | 5.151 | 4.281 | 0.100 | 26.450 |
| | | ADHD | 92 | 6.364 | 7.554 | 0.870 | 60.670 |
| | | Diff (1-2) | — | −1.213 | 6.583 | — | — |
| | Butter | Control | 51 | 17.809 | 24.982 | 0.100 | 150.93 |
| | | ADHD | 92 | 23.143 | 21.663 | 0.740 | 104.00 |
| | | Diff (1-2) | — | −5.334 | 22.895 | — | — |
| | Cabbage | Control | 51 | 5.038 | 6.005 | 0.350 | 37.840 |
| | | ADHD | 92 | 8.829 | 14.699 | 0.740 | 121.38 |
| | | Diff (1-2) | — | −3.791 | 12.338 | — | — |
| | Cane_Sugar | Control | 51 | 15.190 | 10.152 | 3.460 | 50.450 |
| | | ADHD | 92 | 25.370 | 25.407 | 4.050 | 116.64 |
| | | Diff (1-2) | — | −10.180 | 21.287 | — | — |
| | Cantaloupe | Control | 51 | 4.707 | 2.368 | 1.150 | 12.760 |
| | | ADHD | 92 | 11.342 | 20.203 | 1.070 | 134.48 |
| | | Diff (1-2) | — | −6.635 | 16.291 | — | — |
| | Carrot | Control | 51 | 2.702 | 1.549 | 0.100 | 6.940 |
| | | ADHD | 92 | 6.653 | 14.010 | 0.100 | 89.210 |
| | | Diff (1-2) | — | −3.952 | 11.293 | — | — |
| | Cashew | Control | 51 | 8.620 | 13.756 | 0.100 | 81.890 |
| | | ADHD | 92 | 9.483 | 19.123 | 0.610 | 111.06 |
| | | Diff (1-2) | — | −0.863 | 17.410 | — | — |
| | Cauliflower | Control | 51 | 4.203 | 2.424 | 0.430 | 11.770 |
| | | ADHD | 92 | 7.444 | 11.577 | 1.010 | 94.290 |
| | | Diff (1-2) | — | −3.241 | 9.412 | — | — |
| | Celery | Control | 51 | 7.815 | 5.560 | 2.060 | 32.830 |
| | | ADHD | 92 | 11.498 | 13.759 | 2.010 | 86.600 |
| | | Diff (1-2) | — | −3.683 | 11.539 | — | — |
| | Cheddar_Ch_{—} | Control | 51 | 25.261 | 59.384 | 1.530 | 400.00 |
| | | ADHD | 92 | 27.840 | 36.629 | 0.100 | 189.39 |
| | | Diff (1-2) | — | −2.579 | 46.005 | — | — |
| | Chicken | Control | 51 | 14.077 | 8.350 | 2.690 | 50.000 |
| | | ADHD | 92 | 16.458 | 13.609 | 2.270 | 79.130 |
| | | Diff (1-2) | — | −2.381 | 12.010 | — | — |
| | Chili_Pepper | Control | 51 | 7.281 | 6.348 | 0.570 | 32.360 |
| | | ADHD | 92 | 10.595 | 16.487 | 0.870 | 111.31 |
| | | Diff (1-2) | — | −3.314 | 13.774 | — | — |
| | Chocolate | Control | 51 | 13.516 | 6.136 | 3.410 | 30.540 |
| | | ADHD | 92 | 18.291 | 15.883 | 3.950 | 114.88 |
| | | Diff (1-2) | — | −4.775 | 13.272 | — | — |
| | Cinnamon | Control | 51 | 8.315 | 6.347 | 1.490 | 38.800 |
| | | ADHD | 92 | 12.609 | 15.262 | 0.490 | 111.50 |
| | | Diff (1-2) | — | −4.294 | 12.830 | — | — |
| | Clam | Control | 51 | 36.890 | 57.603 | 7.450 | 400.00 |
| | | ADHD | 92 | 31.984 | 32.189 | 3.640 | 213.69 |
| | | Diff (1-2) | — | 4.906 | 42.957 | — | — |
| | Codfish | Control | 51 | 27.484 | 34.270 | 6.170 | 203.91 |
| | | ADHD | 92 | 22.764 | 36.339 | 1.270 | 342.06 |
| | | Diff (1-2) | — | 4.720 | 35.619 | — | — |
| | Coffee | Control | 51 | 34.003 | 55.076 | 6.730 | 400.00 |
| | | ADHD | 92 | 33.999 | 41.052 | 3.850 | 219.51 |
| | | Diff (1-2) | — | 0.004 | 46.512 | — | — |
| | Cola_Nut | Control | 51 | 11.928 | 5.390 | 2.630 | 27.260 |
| | | ADHD | 92 | 13.835 | 7.148 | 1.830 | 33.990 |
| | | Diff (1-2) | — | −1.907 | 6.579 | — | — |
| | Corn | Control | 51 | 7.351 | 5.171 | 2.080 | 27.010 |
| | | ADHD | 92 | 19.225 | 32.677 | 0.530 | 188.39 |
| | | Diff (1-2) | — | −11.874 | 26.431 | — | — |
| | Cottage_Ch_{—} | Control | 51 | 83.139 | 107.442 | 2.120 | 400.00 |
| | | ADHD | 92 | 105.535 | 116.561 | 2.210 | 400.00 |
| | | Diff (1-2) | — | −22.396 | 113.411 | — | — |
| | Cow_Milk | Control | 51 | 65.188 | 90.937 | 1.800 | 400.00 |
| | | ADHD | 92 | 83.361 | 88.266 | 1.100 | 400.00 |
| | | Diff (1-2) | — | −18.173 | 89.222 | — | — |
| | Crab | Control | 51 | 30.367 | 21.673 | 3.770 | 114.37 |
| | | ADHD | 92 | 25.751 | 27.638 | 3.460 | 213.52 |
| | | Diff (1-2) | — | 4.616 | 25.682 | — | — |
| | Cucumber | Control | 51 | 6.560 | 5.111 | 1.270 | 26.500 |
| | | ADHD | 92 | 21.432 | 56.304 | 1.600 | 353.35 |
| | | Diff (1-2) | — | −14.873 | 45.334 | — | — |
| | Egg | Control | 51 | 70.569 | 97.119 | 5.110 | 400.00 |
| | | ADHD | 92 | 109.862 | 120.655 | 3.070 | 400.00 |
| | | Diff (1-2) | — | −39.293 | 112.872 | — | — |
| | Eggplant | Control | 51 | 3.980 | 4.120 | 0.100 | 26.500 |
| | | ADHD | 92 | 9.399 | 23.449 | 0.210 | 155.52 |
| | | Diff (1-2) | — | −5.419 | 18.997 | — | — |
| | Garlic | Control | 51 | 8.615 | 4.650 | 0.100 | 23.410 |
| | | ADHD | 92 | 13.875 | 12.722 | 2.830 | 65.170 |
| | | Diff (1-2) | — | −5.259 | 10.589 | — | — |
| | Goat_Milk | Control | 51 | 12.632 | 28.034 | 0.100 | 149.14 |
| | | ADHD | 92 | 12.332 | 15.717 | 0.100 | 88.430 |
| | | Diff (1-2) | — | 0.300 | 20.931 | — | — |
| | Grape | Control | 51 | 13.069 | 6.123 | 2.350 | 44.190 |
| | | ADHD | 92 | 14.913 | 9.885 | 5.840 | 63.770 |
| | | Diff (1-2) | — | −1.844 | 8.738 | — | — |
| | Grapefruit | Control | 51 | 3.128 | 1.808 | 0.100 | 8.040 |
| | | ADHD | 92 | 5.639 | 8.356 | 0.630 | 54.840 |
| | | Diff (1-2) | — | −2.511 | 6.799 | — | — |
| | Green_Pea | Control | 51 | 5.735 | 5.596 | 0.100 | 27.010 |
| | | ADHD | 92 | 9.805 | 13.113 | 0.100 | 65.560 |
| | | Diff (1-2) | — | −4.070 | 11.049 | — | — |
| | Green_Pepper | Control | 51 | 4.001 | 2.220 | 1.150 | 11.460 |
| | | ADHD | 92 | 6.259 | 8.363 | 0.320 | 54.580 |
| | | Diff (1-2) | — | −2.258 | 6.848 | — | — |
| | Halibut | Control | 51 | 11.307 | 6.425 | 0.850 | 35.370 |
| | | ADHD | 92 | 12.371 | 15.568 | 2.650 | 98.220 |
| | | Diff (1-2) | — | −1.064 | 13.079 | — | — |
| | Honey | Control | 51 | 8.233 | 3.766 | 0.530 | 22.800 |
| | | ADHD | 92 | 10.606 | 7.379 | 2.570 | 48.440 |
| | | Diff (1-2) | — | −2.373 | 6.338 | — | — |
| | Lemon | Control | 51 | 2.559 | 1.273 | 0.100 | 6.270 |
| | | ADHD | 92 | 3.103 | 2.731 | 0.110 | 17.390 |
| | | Diff (1-2) | — | −0.543 | 2.321 | — | — |
| | Lettuce | Control | 51 | 9.676 | 5.181 | 3.730 | 27.090 |
| | | ADHD | 92 | 17.674 | 23.129 | 2.720 | 121.69 |
| | | Diff (1-2) | — | −7.998 | 18.836 | — | — |
| | Lima_Bean | Control | 51 | 4.955 | 4.159 | 0.100 | 25.010 |
| | | ADHD | 92 | 6.487 | 5.863 | 0.880 | 32.820 |
| | | Diff (1-2) | — | −1.531 | 5.321 | — | — |
| | Lobster | Control | 51 | 7.009 | 4.347 | 1.180 | 23.980 |
| | | ADHD | 92 | 9.874 | 16.089 | 1.160 | 118.16 |
| | | Diff (1-2) | — | −2.865 | 13.182 | — | — |
| | Malt | Control | 51 | 13.807 | 7.087 | 1.920 | 31.200 |
| | | ADHD | 92 | 17.057 | 12.192 | 2.810 | 98.820 |
| | | Diff (1-2) | — | −3.250 | 10.665 | — | — |
| | Millet | Control | 51 | 3.882 | 6.158 | 0.100 | 45.890 |
| | | ADHD | 92 | 3.253 | 1.777 | 0.210 | 8.500 |
| | | Diff (1-2) | — | 0.629 | 3.935 | — | — |
| | Mushroom | Control | 51 | 22.699 | 22.937 | 1.490 | 106.22 |
| | | ADHD | 92 | 29.321 | 27.815 | 2.700 | 131.64 |
| | | Diff (1-2) | — | −6.622 | 26.189 | — | — |
| | Mustard | Control | 51 | 5.399 | 2.689 | 0.910 | 11.660 |
| | | ADHD | 92 | 7.816 | 6.990 | 0.120 | 46.630 |
| | | Diff (1-2) | — | −2.417 | 5.839 | — | — |
| | Oat | Control | 51 | 11.120 | 9.562 | 0.100 | 49.540 |
| | | ADHD | 92 | 24.648 | 35.440 | 1.210 | 228.72 |
| | | Diff (1-2) | — | −13.529 | 29.035 | — | — |
| | Olive | Control | 51 | 15.480 | 19.299 | 3.050 | 111.23 |
| | | ADHD | 92 | 30.122 | 61.991 | 3.280 | 400.00 |
| | | Diff (1-2) | — | −14.642 | 51.110 | — | — |
| | Onion | Control | 51 | 9.739 | 10.258 | 1.170 | 70.460 |
| | | ADHD | 92 | 27.033 | 65.323 | 1.800 | 400.00 |
| | | Diff (1-2) | — | −17.293 | 52.833 | — | — |
| | Orange | Control | 51 | 23.728 | 28.880 | 4.170 | 149.43 |
| | | ADHD | 92 | 47.326 | 75.864 | 3.210 | 400.00 |
| | | Diff (1-2) | — | −23.598 | 63.326 | — | — |
| | Oyster | Control | 51 | 44.126 | 34.721 | 9.620 | 168.93 |
| | | ADHD | 92 | 51.409 | 66.452 | 5.780 | 400.00 |
| | | Diff (1-2) | — | −7.283 | 57.249 | — | — |
| | Parsley | Control | 51 | 21.958 | 46.256 | 5.990 | 342.33 |
| | | ADHD | 92 | 19.005 | 15.536 | 5.350 | 76.780 |
| | | Diff (1-2) | — | 2.953 | 30.241 | — | — |
| | Peach | Control | 51 | 6.506 | 7.491 | 0.100 | 34.650 |
| | | ADHD | 92 | 23.107 | 66.936 | 0.210 | 400.00 |
| | | Diff (1-2) | — | −16.601 | 53.958 | — | — |
| | Peanut | Control | 51 | 5.445 | 4.274 | 0.100 | 24.230 |
| | | ADHD | 92 | 6.205 | 10.593 | 0.210 | 85.730 |
| | | Diff (1-2) | — | −0.759 | 8.882 | — | — |
| | Pineapple | Control | 51 | 8.460 | 18.977 | 0.100 | 122.86 |
| | | ADHD | 92 | 17.163 | 45.432 | 0.640 | 400.00 |
| | | Diff (1-2) | — | −8.703 | 38.208 | — | — |
| | Pinto_Bean | Control | 51 | 9.830 | 9.653 | 0.210 | 47.920 |
| | | ADHD | 92 | 17.675 | 26.357 | 1.480 | 139.23 |
| | | Diff (1-2) | — | −7.845 | 21.940 | — | — |
| | Pork | Control | 51 | 15.096 | 8.745 | 4.800 | 44.260 |
| | | ADHD | 92 | 14.611 | 15.147 | 2.000 | 111.43 |
| | | Diff (1-2) | — | 0.485 | 13.236 | — | — |
| | Potato | Control | 51 | 8.664 | 2.240 | 4.900 | 14.010 |
| | | ADHD | 92 | 13.359 | 17.362 | 3.120 | 118.58 |
| | | Diff (1-2) | — | −4.696 | 14.012 | — | — |
| | Rice | Control | 51 | 18.985 | 14.969 | 4.900 | 73.100 |
| | | ADHD | 92 | 26.191 | 27.478 | 3.730 | 189.04 |
| | | Diff (1-2) | — | −7.206 | 23.807 | — | — |
| | Rye | Control | 51 | 4.185 | 2.647 | 0.230 | 17.990 |
| | | ADHD | 92 | 6.593 | 7.838 | 0.100 | 54.320 |
| | | Diff (1-2) | — | −2.409 | 6.491 | — | — |
| | Safflower | Control | 51 | 6.557 | 5.364 | 1.620 | 36.650 |
| | | ADHD | 92 | 10.126 | 19.454 | 1.180 | 178.13 |
| | | Diff (1-2) | — | −3.569 | 15.951 | — | — |
| | Salmon | Control | 51 | 13.155 | 11.632 | 3.480 | 68.370 |
| | | ADHD | 92 | 15.231 | 41.326 | 2.200 | 400.00 |
| | | Diff (1-2) | — | −2.076 | 33.914 | — | — |
| | Sardine | Control | 51 | 29.733 | 14.098 | 12.950 | 76.730 |
| | | ADHD | 92 | 30.082 | 19.193 | 6.570 | 112.43 |
| | | Diff (1-2) | — | −0.349 | 17.556 | — | — |
| | Scallop | Control | 51 | 53.504 | 22.302 | 15.620 | 107.71 |
| | | ADHD | 92 | 54.016 | 42.424 | 10.920 | 283.14 |
| | | Diff (1-2) | — | −0.511 | 36.578 | — | — |
| | Sesame | Control | 51 | 91.740 | 91.167 | 6.640 | 400.00 |
| | | ADHD | 92 | 91.778 | 112.395 | 4.760 | 400.00 |
| | | Diff (1-2) | — | −0.039 | 105.358 | — | — |
| | Shrimp | Control | 51 | 31.906 | 31.340 | 5.360 | 151.14 |
| | | ADHD | 92 | 17.770 | 18.162 | 2.540 | 103.29 |
| | | Diff (1-2) | — | 14.136 | 23.689 | — | — |
| | Sole | Control | 51 | 5.010 | 3.858 | 0.230 | 29.090 |
| | | ADHD | 92 | 5.034 | 3.886 | 0.210 | 32.900 |
| | | Diff (1-2) | — | −0.024 | 3.876 | — | — |
| | Soybean | Control | 51 | 14.277 | 10.254 | 4.150 | 51.570 |
| | | ADHD | 92 | 21.073 | 24.100 | 2.750 | 115.37 |
| | | Diff (1-2) | — | −6.796 | 20.301 | — | — |
| | Spinach | Control | 51 | 20.913 | 15.581 | 3.290 | 66.870 |
| | | ADHD | 92 | 19.725 | 19.295 | 5.350 | 124.21 |
| | | Diff (1-2) | — | 1.188 | 18.065 | — | — |
| | Squashes | Control | 51 | 5.696 | 2.997 | 2.050 | 13.840 |
| | | ADHD | 92 | 13.463 | 27.966 | 0.620 | 210.70 |
| | | Diff (1-2) | — | −7.767 | 22.538 | — | — |
| | Strawberry | Control | 51 | 4.585 | 4.755 | 0.110 | 27.900 |
| | | ADHD | 92 | 15.284 | 50.795 | 0.100 | 331.49 |
| | | Diff (1-2) | — | −10.699 | 40.905 | — | — |
| | String_Bean | Control | 51 | 34.495 | 21.114 | 12.540 | 94.210 |
| | | ADHD | 92 | 36.872 | 27.247 | 7.860 | 184.36 |
| | | Diff (1-2) | — | −2.377 | 25.243 | — | — |
| | Sunflower_Sd | Control | 51 | 7.402 | 4.309 | 1.490 | 21.170 |
| | | ADHD | 92 | 8.995 | 6.411 | 1.790 | 33.590 |
| | | Diff (1-2) | — | −1.593 | 5.754 | — | — |
| | Sweet_Pot_{—} | Control | 51 | 13.319 | 8.693 | 4.460 | 53.650 |
| | | ADHD | 92 | 15.735 | 14.552 | 2.520 | 94.140 |
| | | Diff (1-2) | — | −2.416 | 12.785 | — | — |
| | Swiss_Ch_{—} | Control | 51 | 37.893 | 78.801 | 1.490 | 400.00 |
| | | ADHD | 92 | 38.949 | 59.178 | 1.470 | 386.11 |
| | | Diff (1-2) | — | −1.056 | 66.799 | — | — |
| | Tea | Control | 51 | 19.459 | 7.609 | 8.930 | 38.010 |
| | | ADHD | 92 | 26.526 | 16.518 | 9.070 | 118.42 |
| | | Diff (1-2) | — | −7.066 | 14.022 | — | — |
| | Tobacco | Control | 51 | 28.550 | 13.486 | 7.880 | 65.660 |
| | | ADHD | 92 | 38.636 | 32.339 | 9.270 | 196.29 |
| | | Diff (1-2) | — | −10.085 | 27.193 | — | — |
| | Tomato | Control | 51 | 7.412 | 5.926 | 1.920 | 30.760 |
| | | ADHD | 92 | 26.083 | 69.738 | 0.990 | 400.00 |
| | | Diff (1-2) | — | −18.670 | 56.136 | — | — |
| | Trout | Control | 51 | 15.255 | 16.016 | 3.000 | 93.130 |
| | | ADHD | 92 | 15.496 | 41.328 | 2.570 | 400.00 |
| | | Diff (1-2) | — | −0.241 | 34.544 | — | — |
| | Tuna | Control | 51 | 8.130 | 6.362 | 3.050 | 33.880 |
| | | ADHD | 92 | 5.577 | 5.696 | 0.850 | 46.670 |
| | | Diff (1-2) | — | 2.553 | 5.941 | — | — |
| | Turkey | Control | 51 | 11.859 | 5.301 | 4.490 | 28.920 |
| | | ADHD | 92 | 13.482 | 12.293 | 2.670 | 87.480 |
| | | Diff (1-2) | — | −1.623 | 10.368 | — | — |
| | Walnut_Blk | Control | 51 | 19.796 | 13.830 | 5.670 | 79.530 |
| | | ADHD | 92 | 24.730 | 20.952 | 4.530 | 99.510 |
| | | Diff (1-2) | — | −4.934 | 18.739 | — | — |
| | Wheat | Control | 51 | 14.031 | 16.565 | 3.200 | 116.33 |
| | | ADHD | 92 | 26.488 | 47.247 | 2.110 | 400.00 |
| | | Diff (1-2) | — | −12.457 | 39.217 | — | — |
| | Yeast_Baker | Control | 51 | 6.905 | 4.322 | 2.230 | 24.960 |
| | | ADHD | 92 | 14.363 | 32.026 | 1.360 | 230.36 |
| | | Diff (1-2) | — | −7.458 | 25.857 | — | — |
| | Yeast_Brewer | Control | 51 | 9.946 | 8.060 | 1.490 | 37.540 |
| | | ADHD | 92 | 17.777 | 29.355 | 1.790 | 177.52 |
| | | Diff (1-2) | — | −7.830 | 24.066 | — | — |
| | Yogurt | Control | 51 | 19.256 | 34.792 | 0.100 | 223.20 |
| | | ADHD | 92 | 16.536 | 16.856 | 1.380 | 101.94 |
| | | Diff (1-2) | — | 2.720 | 24.751 | — | — |
| MALE | Almond | Control | 81 | 4.955 | 2.457 | 1.600 | 14.840 |
| | | ADHD | 45 | 7.873 | 9.885 | 0.750 | 50.700 |
| | | Diff (1-2) | — | −2.917 | 6.210 | — | — |
| | Amer_Cheese | Control | 81 | 33.623 | 47.729 | 1.710 | 234.20 |
| | | ADHD | 45 | 41.001 | 45.930 | 1.440 | 238.18 |
| | | Diff (1-2) | — | −7.378 | 47.099 | — | — |
| | Apple | Control | 81 | 4.768 | 4.226 | 0.990 | 30.110 |
| | | ADHD | 45 | 14.584 | 59.891 | 0.100 | 400.00 |
| | | Diff (1-2) | — | −9.816 | 35.837 | — | — |
| | Avocado | Control | 81 | 2.950 | 2.086 | 0.200 | 15.510 |
| | | ADHD | 45 | 2.762 | 2.450 | 0.360 | 14.930 |
| | | Diff (1-2) | — | 0.188 | 2.222 | — | — |
| | Banana | Control | 81 | 4.016 | 5.531 | 0.800 | 48.430 |
| | | ADHD | 45 | 5.730 | 10.042 | 0.620 | 57.670 |
| | | Diff (1-2) | — | −1.714 | 7.451 | — | — |
| | Barley | Control | 81 | 9.009 | 35.683 | 1.080 | 324.19 |
| | | ADHD | 45 | 9.982 | 21.334 | 0.240 | 142.39 |
| | | Diff (1-2) | — | −0.974 | 31.352 | — | — |
| | Beef | Control | 81 | 10.820 | 19.739 | 2.370 | 162.33 |
| | | ADHD | 45 | 8.968 | 10.461 | 1.180 | 64.970 |
| | | Diff (1-2) | — | 1.853 | 17.035 | — | — |
| | Blueberry | Control | 81 | 3.790 | 2.258 | 0.880 | 12.560 |
| | | ADHD | 45 | 3.964 | 4.805 | 0.100 | 33.490 |
| | | Diff (1-2) | — | −0.174 | 3.388 | — | — |
| | Broccoli | Control | 81 | 7.175 | 5.133 | 2.100 | 30.730 |
| | | ADHD | 45 | 10.317 | 13.071 | 2.140 | 84.710 |
| | | Diff (1-2) | — | −3.142 | 8.810 | — | — |
| | Buck_Wheat | Control | 81 | 5.548 | 3.014 | 1.670 | 23.700 |
| | | ADHD | 45 | 6.486 | 4.736 | 0.610 | 25.060 |
| | | Diff (1-2) | — | −0.938 | 3.718 | — | — |
| | Butter | Control | 81 | 16.652 | 19.178 | 1.550 | 93.140 |
| | | ADHD | 45 | 33.273 | 31.469 | 0.120 | 117.94 |
| | | Diff (1-2) | — | −16.621 | 24.263 | — | — |
| | Cabbage | Control | 81 | 5.952 | 10.811 | 0.980 | 94.740 |
| | | ADHD | 45 | 13.046 | 34.923 | 1.780 | 230.48 |
| | | Diff (1-2) | — | −7.094 | 22.543 | — | — |
| | Cane_Sugar | Control | 81 | 23.047 | 28.025 | 3.900 | 170.78 |
| | | ADHD | 45 | 30.327 | 54.839 | 3.250 | 360.99 |
| | | Diff (1-2) | — | −7.280 | 39.672 | — | — |
| | Cantaloupe | Control | 81 | 5.879 | 4.368 | 1.960 | 29.570 |
| | | ADHD | 45 | 20.911 | 61.974 | 0.360 | 400.00 |
| | | Diff (1-2) | — | −15.032 | 37.083 | — | — |
| | Carrot | Control | 81 | 4.016 | 3.787 | 1.180 | 27.680 |
| | | ADHD | 45 | 14.156 | 41.503 | 0.480 | 224.76 |
| | | Diff (1-2) | — | −10.140 | 24.909 | — | — |
| | Cashew | Control | 81 | 9.724 | 11.603 | 1.020 | 59.200 |
| | | ADHD | 45 | 9.675 | 13.714 | 0.100 | 78.260 |
| | | Diff (1-2) | — | 0.050 | 12.393 | — | — |
| | Cauliflower | Control | 81 | 4.865 | 3.698 | 1.510 | 24.160 |
| | | ADHD | 45 | 11.647 | 30.698 | 1.800 | 205.18 |
| | | Diff (1-2) | — | −6.781 | 18.526 | — | — |
| | Celery | Control | 81 | 8.967 | 5.476 | 2.950 | 34.790 |
| | | ADHD | 45 | 15.251 | 26.609 | 2.990 | 155.83 |
| | | Diff (1-2) | — | −6.283 | 16.450 | — | — |
| | Cheddar_Ch_{—} | Control | 81 | 26.696 | 45.931 | 1.690 | 283.73 |
| | | ADHD | 45 | 43.425 | 70.376 | 0.600 | 355.84 |
| | | Diff (1-2) | — | −16.729 | 55.843 | — | — |
| | Chicken | Control | 81 | 16.053 | 12.550 | 2.940 | 76.880 |
| | | ADHD | 45 | 14.770 | 10.368 | 4.290 | 45.240 |
| | | Diff (1-2) | — | 1.283 | 11.822 | — | — |
| | Chili_Pepper | Control | 81 | 7.835 | 5.613 | 1.570 | 38.040 |
| | | ADHD | 45 | 10.254 | 11.413 | 1.800 | 54.050 |
| | | Diff (1-2) | — | −2.419 | 8.157 | — | — |
| | Chocolate | Control | 81 | 16.623 | 11.019 | 3.010 | 59.470 |
| | | ADHD | 45 | 16.970 | 12.146 | 4.580 | 49.160 |
| | | Diff (1-2) | — | −0.347 | 11.432 | — | — |
| | Cinnamon | Control | 81 | 9.850 | 7.037 | 1.640 | 40.480 |
| | | ADHD | 45 | 9.821 | 6.585 | 1.810 | 32.670 |
| | | Diff (1-2) | — | 0.029 | 6.880 | — | — |
| | Clam | Control | 81 | 33.566 | 20.277 | 3.190 | 98.480 |
| | | ADHD | 45 | 26.397 | 25.701 | 2.780 | 132.61 |
| | | Diff (1-2) | — | 7.169 | 22.353 | — | — |
| | Codfish | Control | 81 | 25.075 | 33.649 | 6.490 | 277.17 |
| | | ADHD | 45 | 29.288 | 58.990 | 3.900 | 400.00 |
| | | Diff (1-2) | — | −4.212 | 44.331 | — | — |
| | Coffee | Control | 81 | 30.318 | 43.408 | 4.320 | 356.95 |
| | | ADHD | 45 | 33.387 | 40.314 | 3.970 | 237.41 |
| | | Diff (1-2) | — | −3.069 | 42.336 | — | — |
| | Cola_Nut | Control | 81 | 15.243 | 8.049 | 4.080 | 38.820 |
| | | ADHD | 45 | 14.139 | 8.193 | 4.790 | 36.670 |
| | | Diff (1-2) | — | 1.105 | 8.101 | — | — |
| | Corn | Control | 81 | 9.923 | 12.544 | 2.360 | 95.510 |
| | | ADHD | 45 | 11.735 | 10.231 | 2.160 | 53.180 |
| | | Diff (1-2) | — | −1.812 | 11.775 | — | — |
| | Cottage_Ch_{—} | Control | 81 | 76.631 | 102.972 | 1.210 | 400.00 |
| | | ADHD | 45 | 123.448 | 125.633 | 2.290 | 400.00 |
| | | Diff (1-2) | — | −46.817 | 111.542 | — | — |
| | Cow_Milk | Control | 81 | 60.822 | 83.166 | 1.770 | 400.00 |
| | | ADHD | 45 | 93.019 | 93.827 | 2.040 | 400.00 |
| | | Diff (1-2) | — | −32.197 | 87.098 | — | — |
| | Crab | Control | 81 | 32.448 | 37.288 | 4.770 | 299.11 |
| | | ADHD | 45 | 25.297 | 20.928 | 5.170 | 126.85 |
| | | Diff (1-2) | — | 7.151 | 32.442 | — | — |
| | Cucumber | Control | 81 | 8.752 | 8.584 | 1.880 | 61.860 |
| | | ADHD | 45 | 27.909 | 68.764 | 2.530 | 400.00 |
| | | Diff (1-2) | — | −19.157 | 41.538 | — | — |
| | Egg | Control | 81 | 62.505 | 92.408 | 3.780 | 400.00 |
| | | ADHD | 45 | 120.210 | 128.233 | 2.650 | 400.00 |
| | | Diff (1-2) | — | −57.705 | 106.508 | — | — |
| | Eggplant | Control | 81 | 5.045 | 5.910 | 1.370 | 48.790 |
| | | ADHD | 45 | 8.238 | 16.933 | 1.180 | 90.580 |
| | | Diff (1-2) | — | −3.193 | 11.148 | — | — |
| | Garlic | Control | 81 | 11.918 | 9.606 | 3.040 | 52.160 |
| | | ADHD | 45 | 15.901 | 20.797 | 0.230 | 116.36 |
| | | Diff (1-2) | — | −3.983 | 14.595 | — | — |
| | Goat_Milk | Control | 81 | 11.177 | 16.325 | 0.500 | 96.690 |
| | | ADHD | 45 | 14.943 | 17.272 | 0.600 | 90.320 |
| | | Diff (1-2) | — | −3.767 | 16.667 | — | — |
| | Grape | Control | 81 | 15.645 | 5.750 | 8.060 | 47.250 |
| | | ADHD | 45 | 14.363 | 11.715 | 5.650 | 65.050 |
| | | Diff (1-2) | — | 1.282 | 8.368 | — | — |
| | Grapefruit | Control | 81 | 4.255 | 3.961 | 0.810 | 32.910 |
| | | ADHD | 45 | 11.103 | 44.847 | 0.120 | 304.26 |
| | | Diff (1-2) | — | −6.848 | 26.903 | — | — |
| | Green_Pea | Control | 81 | 7.020 | 6.334 | 1.020 | 35.200 |
| | | ADHD | 45 | 12.287 | 18.687 | 1.320 | 87.570 |
| | | Diff (1-2) | — | −5.267 | 12.239 | — | — |
| | Green_Pepper | Control | 81 | 4.715 | 3.714 | 1.660 | 32.330 |
| | | ADHD | 45 | 7.840 | 13.389 | 1.390 | 76.860 |
| | | Diff (1-2) | — | −3.124 | 8.515 | — | — |
| | Halibut | Control | 81 | 14.289 | 15.877 | 4.410 | 135.74 |
| | | ADHD | 45 | 9.302 | 6.073 | 1.440 | 30.940 |
| | | Diff (1-2) | — | 4.987 | 13.256 | — | — |
| | Honey | Control | 81 | 10.351 | 5.111 | 2.730 | 29.820 |
| | | ADHD | 45 | 11.212 | 10.312 | 4.380 | 64.910 |
| | | Diff (1-2) | — | −0.861 | 7.388 | — | — |
| | Lemon | Control | 81 | 3.051 | 2.461 | 0.200 | 20.660 |
| | | ADHD | 45 | 3.049 | 4.172 | 0.100 | 28.260 |
| | | Diff (1-2) | — | 0.002 | 3.175 | — | — |
| | Lettuce | Control | 81 | 12.815 | 7.663 | 3.730 | 39.970 |
| | | ADHD | 45 | 22.311 | 39.582 | 4.060 | 245.35 |
| | | Diff (1-2) | — | −9.497 | 24.369 | — | — |
| | Lima_Bean | Control | 81 | 6.294 | 5.249 | 1.550 | 35.110 |
| | | ADHD | 45 | 8.261 | 10.797 | 1.440 | 64.200 |
| | | Diff (1-2) | — | −1.967 | 7.690 | — | — |
| | Lobster | Control | 81 | 9.454 | 6.640 | 1.310 | 41.980 |
| | | ADHD | 45 | 8.113 | 5.548 | 1.920 | 27.520 |
| | | Diff (1-2) | — | 1.341 | 6.274 | — | — |
| | Malt | Control | 81 | 15.173 | 8.267 | 2.550 | 51.290 |
| | | ADHD | 45 | 16.614 | 8.000 | 4.680 | 42.770 |
| | | Diff (1-2) | — | −1.441 | 8.173 | — | — |
| | Millet | Control | 81 | 4.065 | 4.304 | 1.440 | 40.360 |
| | | ADHD | 45 | 3.069 | 1.661 | 1.090 | 10.770 |
| | | Diff (1-2) | — | 0.996 | 3.596 | — | — |
| | Mushroom | Control | 81 | 27.234 | 27.375 | 2.820 | 118.76 |
| | | ADHD | 45 | 31.311 | 37.013 | 2.170 | 165.04 |
| | | Diff (1-2) | — | −4.076 | 31.139 | — | — |
| | Mustard | Control | 81 | 6.992 | 4.300 | 1.950 | 30.770 |
| | | ADHD | 45 | 9.070 | 6.716 | 2.350 | 27.670 |
| | | Diff (1-2) | — | −2.078 | 5.285 | — | — |
| | Oat | Control | 81 | 18.201 | 20.144 | 1.180 | 88.430 |
| | | ADHD | 45 | 24.258 | 44.906 | 0.240 | 289.44 |
| | | Diff (1-2) | — | −6.057 | 31.263 | — | — |
| | Olive | Control | 81 | 17.589 | 31.696 | 3.550 | 281.30 |
| | | ADHD | 45 | 32.810 | 71.753 | 3.760 | 400.00 |
| | | Diff (1-2) | — | −15.221 | 49.750 | — | — |
| | Onion | Control | 81 | 13.450 | 23.822 | 2.270 | 210.93 |
| | | ADHD | 45 | 21.552 | 43.799 | 2.260 | 288.97 |
| | | Diff (1-2) | — | −8.101 | 32.355 | — | — |
| | Orange | Control | 81 | 26.423 | 37.325 | 2.820 | 314.77 |
| | | ADHD | 45 | 52.303 | 86.202 | 6.980 | 400.00 |
| | | Diff (1-2) | — | −25.880 | 59.460 | — | — |
| | Oyster | Control | 81 | 49.593 | 42.026 | 7.660 | 250.39 |
| | | ADHD | 45 | 51.189 | 40.645 | 2.990 | 145.00 |
| | | Diff (1-2) | — | −1.596 | 41.542 | — | — |
| | Parsley | Control | 81 | 17.745 | 7.651 | 5.300 | 59.620 |
| | | ADHD | 45 | 18.454 | 19.955 | 7.580 | 120.17 |
| | | Diff (1-2) | — | −0.710 | 13.381 | — | — |
| | Peach | Control | 81 | 10.413 | 10.155 | 1.910 | 53.130 |
| | | ADHD | 45 | 29.666 | 69.796 | 1.710 | 390.11 |
| | | Diff (1-2) | — | −19.253 | 42.369 | — | — |
| | Peanut | Control | 81 | 5.730 | 9.912 | 1.220 | 89.270 |
| | | ADHD | 45 | 7.088 | 9.960 | 0.210 | 61.860 |
| | | Diff (1-2) | — | −1.358 | 9.929 | — | — |
| | Pineapple | Control | 81 | 12.433 | 44.326 | 1.660 | 400.00 |
| | | ADHD | 45 | 24.370 | 71.590 | 1.680 | 400.00 |
| | | Diff (1-2) | — | −11.937 | 55.554 | — | — |
| | Pinto_Bean | Control | 81 | 9.371 | 6.088 | 2.000 | 33.950 |
| | | ADHD | 45 | 18.432 | 47.574 | 0.840 | 326.50 |
| | | Diff (1-2) | — | −9.061 | 28.758 | — | — |
| | Pork | Control | 81 | 16.675 | 14.641 | 4.200 | 89.420 |
| | | ADHD | 45 | 13.623 | 14.044 | 3.990 | 95.400 |
| | | Diff (1-2) | — | 3.052 | 14.432 | — | — |
| | Potato | Control | 81 | 12.243 | 8.339 | 4.920 | 75.770 |
| | | ADHD | 45 | 19.614 | 42.471 | 3.490 | 238.41 |
| | | Diff (1-2) | — | −7.372 | 26.171 | — | — |
| | Rice | Control | 81 | 24.230 | 16.518 | 4.810 | 79.620 |
| | | ADHD | 45 | 26.083 | 23.639 | 4.400 | 104.65 |
| | | Diff (1-2) | — | −1.852 | 19.347 | — | — |
| | Rye | Control | 81 | 5.122 | 3.376 | 1.570 | 23.490 |
| | | ADHD | 45 | 8.482 | 9.991 | 0.100 | 42.730 |
| | | Diff (1-2) | — | −3.360 | 6.540 | — | — |
| | Safflower | Control | 81 | 7.553 | 4.019 | 2.450 | 27.490 |
| | | ADHD | 45 | 11.774 | 16.638 | 2.070 | 99.350 |
| | | Diff (1-2) | — | −4.221 | 10.423 | — | — |
| | Salmon | Control | 81 | 16.308 | 15.973 | 0.100 | 136.52 |
| | | ADHD | 45 | 11.480 | 12.387 | 0.370 | 66.780 |
| | | Diff (1-2) | — | 4.827 | 14.800 | — | — |
| | Sardine | Control | 81 | 33.100 | 14.613 | 7.840 | 87.490 |
| | | ADHD | 45 | 34.280 | 34.250 | 3.530 | 223.07 |
| | | Diff (1-2) | — | −1.181 | 23.537 | — | — |
| | Scallop | Control | 81 | 50.308 | 23.098 | 11.060 | 116.33 |
| | | ADHD | 45 | 55.161 | 42.723 | 6.980 | 162.20 |
| | | Diff (1-2) | — | −4.854 | 31.494 | — | — |
| | Sesame | Control | 81 | 73.448 | 87.621 | 3.430 | 400.00 |
| | | ADHD | 45 | 98.254 | 111.756 | 2.880 | 400.00 |
| | | Diff (1-2) | — | −24.806 | 96.876 | — | — |
| | Shrimp | Control | 81 | 34.185 | 40.052 | 2.930 | 272.28 |
| | | ADHD | 45 | 19.372 | 14.056 | 4.160 | 57.840 |
| | | Diff (1-2) | — | 14.812 | 33.242 | — | — |
| | Sole | Control | 81 | 5.290 | 2.521 | 2.240 | 20.370 |
| | | ADHD | 45 | 4.138 | 1.985 | 0.370 | 10.540 |
| | | Diff (1-2) | — | 1.152 | 2.344 | — | — |
| | Soybean | Control | 81 | 16.814 | 12.312 | 3.480 | 81.380 |
| | | ADHD | 45 | 19.752 | 19.790 | 4.900 | 110.99 |
| | | Diff (1-2) | — | −2.938 | 15.387 | — | — |
| | Spinach | Control | 81 | 14.620 | 6.503 | 5.380 | 40.130 |
| | | ADHD | 45 | 22.821 | 33.693 | 4.590 | 232.80 |
| | | Diff (1-2) | — | −8.201 | 20.739 | — | — |
| | Squashes | Control | 81 | 7.200 | 4.790 | 2.260 | 24.680 |
| | | ADHD | 45 | 19.699 | 52.168 | 2.460 | 332.85 |
| | | Diff (1-2) | — | −12.499 | 31.313 | — | — |
| | Strawberry | Control | 81 | 5.072 | 4.417 | 1.000 | 29.160 |
| | | ADHD | 45 | 15.664 | 59.370 | 0.730 | 400.00 |
| | | Diff (1-2) | — | −10.591 | 35.543 | — | — |
| | String_Bean | Control | 81 | 37.257 | 22.322 | 7.890 | 146.17 |
| | | ADHD | 45 | 45.077 | 59.336 | 7.050 | 400.00 |
| | | Diff (1-2) | — | −7.820 | 39.633 | — | — |
| | Sunflower_Sd | Control | 81 | 8.566 | 5.303 | 2.450 | 31.260 |
| | | ADHD | 45 | 11.785 | 17.504 | 3.250 | 119.64 |
| | | Diff (1-2) | — | −3.220 | 11.264 | — | — |
| | Sweet_Pot_{—} | Control | 81 | 17.535 | 13.698 | 4.100 | 74.660 |
| | | ADHD | 45 | 16.360 | 17.323 | 3.100 | 91.230 |
| | | Diff (1-2) | — | 1.175 | 15.084 | — | — |
| | Swiss_Ch_{—} | Control | 81 | 35.608 | 58.963 | 2.010 | 299.50 |
| | | ADHD | 45 | 54.448 | 81.068 | 2.030 | 357.47 |
| | | Diff (1-2) | — | −18.840 | 67.638 | — | — |
| | Tea | Control | 81 | 23.966 | 9.868 | 7.620 | 46.400 |
| | | ADHD | 45 | 28.329 | 23.408 | 8.760 | 159.87 |
| | | Diff (1-2) | — | −4.363 | 16.039 | — | — |
| | Tobacco | Control | 81 | 36.231 | 21.642 | 8.830 | 125.93 |
| | | ADHD | 45 | 42.184 | 34.190 | 8.580 | 211.54 |
| | | Diff (1-2) | — | −5.953 | 26.776 | — | — |
| | Tomato | Control | 81 | 9.199 | 6.995 | 2.320 | 40.930 |
| | | ADHD | 45 | 30.259 | 70.799 | 2.530 | 387.96 |
| | | Diff (1-2) | — | −21.059 | 42.546 | — | — |
| | Trout | Control | 81 | 14.686 | 9.991 | 3.220 | 83.960 |
| | | ADHD | 45 | 13.454 | 14.761 | 3.330 | 85.230 |
| | | Diff (1-2) | — | 1.233 | 11.904 | — | — |
| | Tuna | Control | 81 | 8.305 | 6.512 | 2.110 | 39.020 |
| | | ADHD | 45 | 12.132 | 25.126 | 2.120 | 122.92 |
| | | Diff (1-2) | — | −3.827 | 15.855 | — | — |
| | Turkey | Control | 81 | 14.012 | 11.117 | 4.080 | 65.180 |
| | | ADHD | 45 | 13.009 | 8.626 | 4.040 | 36.080 |
| | | Diff (1-2) | — | 1.003 | 10.302 | — | — |
| | Walnut_Blk | Control | 81 | 20.821 | 10.403 | 5.680 | 58.470 |
| | | ADHD | 45 | 28.055 | 32.043 | 7.160 | 149.93 |
| | | Diff (1-2) | — | −7.234 | 20.836 | — | — |
| | Wheat | Control | 81 | 13.359 | 10.034 | 3.240 | 71.930 |
| | | ADHD | 45 | 43.230 | 74.407 | 4.300 | 400.00 |
| | | Diff (1-2) | — | −29.871 | 45.050 | — | — |
| | Yeast_Baker | Control | 81 | 12.471 | 20.370 | 2.070 | 123.35 |
| | | ADHD | 45 | 12.101 | 16.201 | 1.390 | 77.180 |
| | | Diff (1-2) | — | 0.371 | 18.995 | — | — |
| | Yeast_Brewer | Control | 81 | 15.903 | 21.144 | 2.640 | 130.89 |
| | | ADHD | 45 | 18.229 | 26.775 | 2.030 | 121.54 |
| | | Diff (1-2) | — | −2.326 | 23.298 | — | — |
| | Yogurt | Control | 81 | 15.650 | 16.294 | 3.000 | 73.200 |
| | | ADHD | 45 | 18.662 | 17.105 | 1.280 | 76.030 |
| | | Diff (1-2) | — | −3.012 | 16.587 | — | — |

TABLE 4
Upper Quantiles of ELISA Signal Scores among Control Subjects
as Candidates for Test Cutpoints in Determining “Positive”
or “Negative” Top 26 Foods Ranked by Descending
order of Discriminatory Ability using Permutation Test
ADD/ADHD Subjects vs. Controls

| | | | Cutpoint | |
|---|---|---|---|---|
| | | | 90th | 95th |
| Food Ranking | Food | Sex | percentile | percentile |
| 1 | Cantaloupe | FEMALE | 7.774 | 9.885 |
| | | MALE | 10.247 | 14.980 |
| 2 | Wheat | FEMALE | 22.676 | 40.002 |
| | | MALE | 23.891 | 30.922 |
| 3 | Tomato | FEMALE | 12.757 | 20.137 |
| | | MALE | 16.321 | 23.825 |
| 4 | Cucumber | FEMALE | 12.269 | 17.960 |
| | | MALE | 16.150 | 22.827 |
| 5 | Squashes | FEMALE | 10.267 | 11.963 |
| | | MALE | 13.574 | 18.485 |
| 6 | Almond | FEMALE | 6.392 | 7.269 |
| | | MALE | 8.212 | 10.337 |
| 7 | Egg | FEMALE | 188.62 | 321.65 |
| | | MALE | 145.49 | 298.13 |
| 8 | Cauliflower | FEMALE | 7.594 | 8.954 |
| | | MALE | 7.799 | 12.288 |
| 9 | Pinto_Bean | FEMALE | 20.059 | 32.885 |
| | | MALE | 17.813 | 21.226 |
| 10 | Broccoli | FEMALE | 9.069 | 12.037 |
| | | MALE | 13.200 | 18.126 |
| 11 | Orange | FEMALE | 59.347 | 90.581 |
| | | MALE | 47.525 | 68.415 |
| 12 | Butter | FEMALE | 40.002 | 67.588 |
| | | MALE | 43.433 | 60.984 |
| 13 | Corn | FEMALE | 13.161 | 19.357 |
| | | MALE | 17.173 | 28.120 |
| 14 | Lettuce | FEMALE | 16.940 | 21.280 |
| | | MALE | 23.239 | 30.306 |
| 15 | Rye | FEMALE | 6.198 | 8.944 |
| | | MALE | 8.399 | 11.978 |
| 16 | Peach | FEMALE | 16.434 | 25.262 |
| | | MALE | 22.310 | 32.987 |
| 17 | Green_Pea | FEMALE | 13.221 | 18.595 |
| | | MALE | 14.660 | 21.095 |
| 18 | Carrot | FEMALE | 5.055 | 6.010 |
| | | MALE | 6.600 | 9.684 |
| 19 | Tea | FEMALE | 29.837 | 33.245 |
| | | MALE | 37.540 | 42.112 |
| 20 | Mustard | FEMALE | 9.607 | 10.799 |
| | | MALE | 11.762 | 14.581 |
| 21 | Strawberry | FEMALE | 7.900 | 14.127 |
| | | MALE | 9.448 | 14.125 |
| 22 | Celery | FEMALE | 14.210 | 19.974 |
| | | MALE | 15.168 | 19.728 |
| 23 | Green_Pepper | FEMALE | 6.912 | 8.275 |
| | | MALE | 7.092 | 9.348 |
| 24 | Garlic | FEMALE | 15.016 | 17.352 |
| | | MALE | 24.205 | 34.240 |
| 25 | Oat | FEMALE | 22.743 | 27.596 |
| | | MALE | 51.597 | 63.749 |
| 26 | Onion | FEMALE | 18.635 | 26.190 |
| | | MALE | 26.971 | 32.220 |
| 27 | Banana | FEMALE | 5.276 | 8.990 |
| | | MALE | 6.632 | 10.083 |
| 28 | Eggplant | FEMALE | 7.193 | 11.328 |
| | | MALE | 7.631 | 12.926 |
| 29 | Cabbage | FEMALE | 8.484 | 15.011 |
| | | MALE | 9.596 | 16.710 |
| 30 | Safflower | FEMALE | 10.328 | 16.067 |
| | | MALE | 11.820 | 14.861 |
| 31 | Olive | FEMALE | 27.849 | 55.153 |
| | | MALE | 28.220 | 44.995 |
| 32 | Cottage_Ch_{—} | FEMALE | 244.09 | 354.86 |
| | | MALE | 225.78 | 341.05 |
| 33 | Grapefruit | FEMALE | 5.731 | 6.772 |
| | | MALE | 7.127 | 10.165 |
| 34 | Walnut_Blk | FEMALE | 38.596 | 49.729 |
| | | MALE | 35.760 | 41.973 |
| 35 | Cow_Milk | FEMALE | 155.08 | 281.94 |
| | | MALE | 149.48 | 244.73 |
| 36 | Soybean | FEMALE | 27.522 | 38.061 |
| | | MALE | 31.249 | 41.905 |
| 37 | Chili_Pepper | FEMALE | 15.566 | 21.521 |
| | | MALE | 14.808 | 18.731 |

TABLE 5A

| ADHD POPULATION | | NON-ADHD POPULATION | |
|---|---|---|---|
| | # of Positive | | # of Positive |
| | Results Based | | Results Based |
| | on 90th | | on 90th |
| Sample ID | Percentile | Sample ID | Percentile |
| BRH1227838 | 10 | BRH1244994 | 0 |
| BRH1227840 | 2 | BRH1244995 | 0 |
| BRH1227841 | 13 | BRH1244996 | 1 |
| BRH1227842 | 2 | BRH1244997 | 1 |
| BRH1227843 | 6 | BRH1244998 | 2 |
| BRH1227844 | 13 | BRH1244999 | 0 |
| BRH1227846 | 13 | BRH1245000 | 2 |
| BRH1243697 | 0 | BRH1245001 | 0 |
| BRH1243698 | 8 | BRH1245002 | 1 |
| BRH1243699 | 2 | BRH1245004 | 0 |
| KH16-13285 | 0 | BRH1245007 | 0 |
| KH16-14189 | 1 | BRH1245008 | 1 |
| KH16-14190 | 1 | BRH1245009 | 1 |
| KH16-14586 | 0 | BRH1245010 | 5 |
| KH16-14587 | 0 | BRH1245011 | 2 |
| KH16-16347 | 10 | BRH1245014 | 0 |
| KH16-21226 | 3 | BRH1245015 | 1 |
| KH17-4120 | 3 | BRH1245018 | 1 |
| KH17-4757 | 1 | BRH1245019 | 1 |
| KH17-5055 | 0 | BRH1245022 | 11 |
| KH17-5056 | 0 | BRH1245023 | 1 |
| DLS17-012889 | 3 | BRH1245024 | 2 |
| KH17-00912 | 9 | BRH1245026 | 2 |
| KH17-1238 | 20 | BRH1245029 | 1 |
| KH17-2452 | 10 | BRH1245030 | 0 |
| KH17-2453 | 20 | BRH1245031 | 2 |
| KH17-2454 | 0 | BRH1245032 | 1 |
| KH17-2676 | 4 | BRH1245033 | 2 |
| KH17-4038 | 1 | BRH1245035 | 0 |
| KH17-4039 | 10 | BRH1245037 | 0 |
| DLS16-69541 | 0 | BRH1245038 | 0 |
| 160904AAC0045 | 3 | BRH1245039 | 9 |
| 160904AAC0046 | 20 | BRH1245040 | 5 |
| 160904AAC0047 | 5 | BRH1245041 | 3 |
| 160904AAC0048 | 2 | BRH1267328 | 12 |
| 160904AAC0049 | 10 | BRH1267329 | 2 |
| 160904AAC0050 | 0 | BRH1267330 | 0 |
| 160904AAC0051 | 5 | BRH1267332 | 0 |
| 160904AAC0021 | 9 | BRH1267333 | 0 |
| 160904AAC0023 | 1 | BRH1267334 | 9 |
| 160904AAC0025 | 10 | BRH1267335 | 0 |
| 160904AAC0026 | 6 | BRH1267337 | 2 |
| 160904AAC0028 | 3 | BRH1267338 | 0 |
| 160904AAC0037 | 4 | BRH1267339 | 2 |
| 160904AAC0038 | 0 | BRH1267340 | 6 |
| 160904AAC0039 | 8 | BRH1267341 | 0 |
| 160904AAC0040 | 9 | BRH1267343 | 5 |
| 160904AAC0042 | 3 | BRH1267345 | 0 |
| 160904AAC0012 | 1 | BRH1267346 | 0 |
| 160904AAC0013 | 1 | BRH1267347 | 0 |
| 160904AAC0014 | 2 | BRH1267349 | 1 |
| 160904AAC0015 | 7 | BRH1244900 | 1 |
| 160904AAC0016 | 20 | BRH1244901 | 4 |
| 160904AAC0017 | 1 | BRH1244902 | 0 |
| 160904AAC0018 | 0 | BRH1244903 | 0 |
| 160904AAC0019 | 4 | BRH1244904 | 1 |
| 160904AAC0020 | 5 | BRH1244905 | 0 |
| 160880AAB0022 | 2 | BRH1244906 | 5 |
| 160880AAB0024 | 6 | BRH1244907 | 0 |
| 160880AAB0025 | 0 | BRH1244908 | 2 |
| 160880AAB0026 | 18 | BRH1244909 | 1 |
| 160880AAB0028 | 11 | BRH1244910 | 2 |
| 160880AAB0029 | 0 | BRH1244911 | 0 |
| 160904AAC0009 | 0 | BRH1244912 | 2 |
| 160904AAC0010 | 6 | BRH1244913 | 0 |
| 160904AAC0001 | 0 | BRH1244914 | 3 |
| 160904AAC0003 | 0 | BRH1244915 | 0 |
| 160904AAC0004 | 7 | BRH1244916 | 12 |
| 160904AAC0005 | 2 | BRH1244917 | 7 |
| 160880AAB0031 | 3 | BRH1244918 | 0 |
| 160880AAB0032 | 2 | BRH1244920 | 3 |
| 160880AAB0033 | 3 | BRH1244921 | 1 |
| 160880AAB0034 | 20 | BRH1244922 | 12 |
| 160880AAB0002 | 13 | BRH1244923 | 1 |
| 160880AAB0003 | 9 | BRH1244924 | 1 |
| 160880AAB0004 | 0 | BRH1244925 | 1 |
| 160880AAB0006 | 0 | BRH1244926 | 8 |
| 160880AAB0007 | 3 | BRH1244928 | 1 |
| 160880AAB0009 | 3 | BRH1244929 | 3 |
| 160880AAB0010 | 8 | BRH1244931 | 1 |
| 160880AAB0011 | 6 | BRH1244932 | 2 |
| 160880AAB0013 | 1 | BRH1244933 | 3 |
| 160880AAB0014 | 0 | BRH1244934 | 3 |
| 160880AAB0015 | 0 | BRH1244938 | 3 |
| 160880AAB0016 | 6 | BRH1244939 | 0 |
| 160880AAB0017 | 0 | BRH1244940 | 0 |
| 160880AAB0018 | 0 | BRH1244941 | 1 |
| 11782002 | 2 | BRH1244942 | 4 |
| 11827629 | 1 | BRH1244943 | 2 |
| 11828836 | 0 | BRH1244944 | 8 |
| 12018682 | 0 | BRH1244945 | 1 |
| 12035395 | 0 | BRH1244946 | 6 |
| BRH1227836 | 4 | BRH1244947 | 0 |
| BRH1227837 | 1 | BRH1244948 | 0 |
| BRH1227839 | 0 | BRH1244949 | 2 |
| BRH1227845 | 13 | BRH1244950 | 0 |
| KH16-13576 | 3 | BRH1244951 | 0 |
| KH16-16348 | 1 | BRH1244952 | 0 |
| KH16-18260 | 0 | BRH1244953 | 1 |
| KH16-18261 | 6 | BRH1244954 | 0 |
| KH16-18262 | 0 | BRH1244956 | 19 |
| KH16-19420 | 3 | BRH1244959 | 1 |
| KH17-4040 | 0 | BRH1244960 | 0 |
| KH17-4613 | 5 | BRH1244961 | 0 |
| KH17-4614 | 5 | BRH1244962 | 1 |
| DLS17-012888 | 0 | BRH1244963 | 0 |
| KH16-19421 | 3 | BRH1244964 | 4 |
| KH17-00072 | 0 | BRH1244965 | 1 |
| KH17-00073 | 8 | BRH1244967 | 0 |
| KH17-1237 | 1 | BRH1244969 | 1 |
| 160904AAC0043 | 7 | BRH1244970 | 3 |
| 160904AAC0044 | 0 | BRH1244971 | 0 |
| 160904AAC0052 | 3 | BRH1244972 | 1 |
| 160904AAC0022 | 8 | BRH1244973 | 3 |
| 160904AAC0024 | 6 | BRH1244974 | 0 |
| 160904AAC0027 | 18 | BRH1244975 | 0 |
| 160904AAC0029 | 1 | BRH1244976 | 0 |
| 160904AAC0030 | 7 | BRH1244977 | 0 |
| 160904AAC0031 | 1 | BRH1244979 | 0 |
| 160904AAC0032 | 1 | BRH1244980 | 1 |
| 160904AAC0033 | 6 | BRH1244981 | 1 |
| 160904AAC0034 | 3 | BRH1244982 | 1 |
| 160904AAC0035 | 16 | BRH1244983 | 0 |
| 160904AAC0036 | 3 | BRH1244985 | 0 |
| 160904AAC0041 | 4 | BRH1244987 | 0 |
| 160904AAC0011 | 1 | BRH1244988 | 5 |
| 160880AAB0021 | 1 | BRH1244991 | 0 |
| 160880AAB0023 | 2 | BRH1244992 | 1 |
| 160880AAB0027 | 4 | BRH1267320 | 1 |
| 160904AAC0008 | 0 | BRH1267322 | 7 |
| 160904AAC0002 | 3 | BRH1267323 | 0 |
| 160880AAB0030 | 19 | BRH1267325 | 3 |
| 160880AAB0008 | 3 | No of | 132 |
| | | Observations | |
| 160880AAB0012 | 1 | Average Number | 2.0 |
| 11827735 | 2 | Median Number | 1 |
| 11940652 | 2 | # of Patients | 51 |
| | | w/0 Pos Results | |
| 12080010 | 3 | % Subjects w/0 | 38.6 |
| | | pos results | |
| No of | 137 | | |
| Observations | | | |
| Average Number | 4.5 | | |
| Median Number | 3 | | |
| # of Patients | 33 | | |
| w/0 Pos Results | | | |
| % Subjects w/0 | 24.1 | | |
| pos results | | | |

TABLE 5B

| ADHD POPULATION | | NON-ADHD POPULATION | |
|---|---|---|---|
| | # of Positive | | # of Positive |
| | Results Based | | Results Based |
| | on 95th | | on 95th |
| Sample ID | Percentile | Sample ID | Percentile |
| BRH1227838 | 5 | BRH1244994 | 0 |
| BRH1227840 | 1 | BRH1244995 | 0 |
| BRH1227841 | 7 | BRH1244996 | 0 |
| BRH1227842 | 2 | BRH1244997 | 0 |
| BRH1227843 | 2 | BRH1244998 | 1 |
| BRH1227844 | 5 | BRH1244999 | 0 |
| BRH1227846 | 7 | BRH1245000 | 0 |
| BRH1243697 | 0 | BRH1245001 | 0 |
| BRH1243698 | 3 | BRH1245002 | 1 |
| BRH1243699 | 0 | BRH1245004 | 0 |
| KH16-13285 | 0 | BRH1245007 | 0 |
| KH16-14189 | 0 | BRH1245008 | 0 |
| KH16-14190 | 1 | BRH1245009 | 0 |
| KH16-14586 | 0 | BRH1245010 | 3 |
| KH16-14587 | 0 | BRH1245011 | 2 |
| KH16-16347 | 6 | BRH1245014 | 0 |
| KH16-21226 | 1 | BRH1245015 | 0 |
| KH17-4120 | 2 | BRH1245018 | 1 |
| KH17-4757 | 1 | BRH1245019 | 1 |
| KH17-5055 | 0 | BRH1245022 | 6 |
| KH17-5056 | 0 | BRH1245023 | 1 |
| DLS17-012889 | 1 | BRH1245024 | 1 |
| KH17-00912 | 7 | BRH1245026 | 2 |
| KH17-1238 | 20 | BRH1245029 | 1 |
| KH17-2452 | 7 | BRH1245030 | 0 |
| KH17-2453 | 20 | BRH1245031 | 1 |
| KH17-2454 | 0 | BRH1245032 | 0 |
| KH17-2676 | 3 | BRH1245033 | 0 |
| KH17-4038 | 0 | BRH1245035 | 0 |
| KH17-4039 | 4 | BRH1245037 | 0 |
| DLS16-69541 | 0 | BRH1245038 | 0 |
| 160904AAC0045 | 2 | BRH1245039 | 7 |
| 160904AAC0046 | 16 | BRH1245040 | 3 |
| 160904AAC0047 | 4 | BRH1245041 | 1 |
| 160904AAC0048 | 0 | BRH1267328 | 4 |
| 160904AAC0049 | 7 | BRH1267329 | 0 |
| 160904AAC0050 | 0 | BRH1267330 | 0 |
| 160904AAC0051 | 2 | BRH1267332 | 0 |
| 160904AAC0021 | 6 | BRH1267333 | 0 |
| 160904AAC0023 | 0 | BRH1267334 | 4 |
| 160904AAC0025 | 8 | BRH1267335 | 0 |
| 160904AAC0026 | 5 | BRH1267337 | 1 |
| 160904AAC0028 | 3 | BRH1267338 | 0 |
| 160904AAC0037 | 0 | BRH1267339 | 0 |
| 160904AAC0038 | 0 | BRH1267340 | 6 |
| 160904AAC0039 | 3 | BRH1267341 | 0 |
| 160904AAC0040 | 5 | BRH1267343 | 2 |
| 160904AAC0042 | 1 | BRH1267345 | 0 |
| 160904AAC0012 | 0 | BRH1267346 | 0 |
| 160904AAC0013 | 1 | BRH1267347 | 0 |
| 160904AAC0014 | 2 | BRH1267349 | 1 |
| 160904AAC0015 | 5 | BRH1244900 | 1 |
| 160904AAC0016 | 20 | BRH1244901 | 1 |
| 160904AAC0017 | 0 | BRH1244902 | 0 |
| 160904AAC0018 | 0 | BRH1244903 | 0 |
| 160904AAC0019 | 2 | BRH1244904 | 1 |
| 160904AAC0020 | 2 | BRH1244905 | 0 |
| 160880AAB0022 | 0 | BRH1244906 | 3 |
| 160880AAB0024 | 4 | BRH1244907 | 0 |
| 160880AAB0025 | 0 | BRH1244908 | 2 |
| 160880AAB0026 | 13 | BRH1244909 | 1 |
| 160880AAB0028 | 10 | BRH1244910 | 0 |
| 160880AAB0029 | 0 | BRH1244911 | 0 |
| 160904AAC0009 | 0 | BRH1244912 | 0 |
| 160904AAC0010 | 5 | BRH1244913 | 0 |
| 160904AAC0001 | 0 | BRH1244914 | 1 |
| 160904AAC0003 | 0 | BRH1244915 | 0 |
| 160904AAC0004 | 3 | BRH1244916 | 6 |
| 160904AAC0005 | 1 | BRH1244917 | 3 |
| 160880AAB0031 | 1 | BRH1244918 | 0 |
| 160880AAB0032 | 0 | BRH1244920 | 3 |
| 160880AAB0033 | 0 | BRH1244921 | 0 |
| 160880AAB0034 | 17 | BRH1244922 | 9 |
| 160880AAB0002 | 6 | BRH1244923 | 0 |
| 160880AAB0003 | 4 | BRH1244924 | 1 |
| 160880AAB0004 | 0 | BRH1244925 | 1 |
| 160880AAB0006 | 0 | BRH1244926 | 7 |
| 160880AAB0007 | 3 | BRH1244928 | 1 |
| 160880AAB0009 | 1 | BRH1244929 | 2 |
| 160880AAB0010 | 4 | BRH1244931 | 1 |
| 160880AAB0011 | 1 | BRH1244932 | 1 |
| 160880AAB0013 | 1 | BRH1244933 | 2 |
| 160880AAB0014 | 0 | BRH1244934 | 2 |
| 160880AAB0015 | 0 | BRH1244938 | 1 |
| 160880AAB0016 | 3 | BRH1244939 | 0 |
| 160880AAB0017 | 0 | BRH1244940 | 0 |
| 160880AAB0018 | 0 | BRH1244941 | 0 |
| 11782002 | 1 | BRH1244942 | 3 |
| 11827629 | 1 | BRH1244943 | 0 |
| 11828836 | 0 | BRH1244944 | 1 |
| 12018682 | 0 | BRH1244945 | 1 |
| 12035395 | 0 | BRH1244946 | 4 |
| BRH1227836 | 2 | BRH1244947 | 0 |
| BRH1227837 | 0 | BRH1244948 | 0 |
| BRH1227839 | 0 | BRH1244949 | 0 |
| BRH1227845 | 4 | BRH1244950 | 0 |
| KH16-13576 | 2 | BRH1244951 | 0 |
| KH16-16348 | 0 | BRH1244952 | 0 |
| KH16-18260 | 0 | BRH1244953 | 1 |
| KH16-18261 | 3 | BRH1244954 | 0 |
| KH16-18262 | 0 | BRH1244956 | 10 |
| KH16-19420 | 2 | BRH1244959 | 0 |
| KH17-4040 | 0 | BRH1244960 | 0 |
| KH17-4613 | 5 | BRH1244961 | 0 |
| KH17-4614 | 2 | BRH1244962 | 0 |
| DLS17-012888 | 0 | BRH1244963 | 0 |
| KH16-19421 | 3 | BRH1244964 | 3 |
| KH17-00072 | 0 | BRH1244965 | 0 |
| KH17-00073 | 3 | BRH1244967 | 0 |
| KH17-1237 | 1 | BRH1244969 | 1 |
| 160904AAC0043 | 4 | BRH1244970 | 1 |
| 160904AAC0044 | 0 | BRH1244971 | 0 |
| 160904AAC0052 | 2 | BRH1244972 | 1 |
| 160904AAC0022 | 7 | BRH1244973 | 0 |
| 160904AAC0024 | 0 | BRH1244974 | 0 |
| 160904AAC0027 | 16 | BRH1244975 | 0 |
| 160904AAC0029 | 1 | BRH1244976 | 0 |
| 160904AAC0030 | 3 | BRH1244977 | 0 |
| 160904AAC0031 | 1 | BRH1244979 | 0 |
| 160904AAC0032 | 1 | BRH1244980 | 0 |
| 160904AAC0033 | 3 | BRH1244981 | 0 |
| 160904AAC0034 | 2 | BRH1244982 | 0 |
| 160904AAC0035 | 16 | BRH1244983 | 0 |
| 160904AAC0036 | 1 | BRH1244985 | 0 |
| 160904AAC0041 | 1 | BRH1244987 | 0 |
| 160904AAC0011 | 1 | BRH1244988 | 3 |
| 160880AAB0021 | 0 | BRH1244991 | 0 |
| 160880AAB0023 | 0 | BRH1244992 | 0 |
| 160880AAB0027 | 4 | BRH1267320 | 0 |
| 160904AAC0008 | 0 | BRH1267322 | 0 |
| 160904AAC0002 | 3 | BRH1267323 | 0 |
| 160880AAB0030 | 19 | BRH1267325 | 1 |
| 160880AAB0008 | 2 | No of | 132 |
| | | Observations | |
| 160880AAB0012 | 1 | Average Number | 1.0 |
| 11827735 | 2 | Median Number | 0 |
| 11940652 | 1 | # of Patients | 78 |
| | | w/0 Pos Results | |
| 12080010 | 3 | % Subjects w/0 | 59.1 |
| | | pos results | |
| No of | 137 | | |
| Observations | | | |
| Average Number | 2.9 | | |
| Median Number | 1 | | |
| # of Patients | 49 | | |
| w/0 Pos Results | | | |
| % Subjects w/0 | 35.8 | | |
| pos results | | | |

TABLE 6A
Summary statistics

| | |
|---|---|
| Variable | ADHD_90th_percentile |
| | ADHD 90th percentile |

| Sample size | 137 | |
|---|---|---|
| Lowest value | 0.0000 | |
| Highest value | 20.0000 | |
| Arithmetic mean | 4.4818 | |
| 95% CI for the mean | 3.6002 to 5.3633 | |
| Median | 3.0000 | |
| 95% CI for the median | 2.0000 to 3.0000 | |
| Variance | 27.2221 | |
| Standard deviation | 5.2175 | |
| Relative standard deviation | 1.1642 | (116.42%) |
| Standard error of the mean | 0.4458 | |
| Coefficient of Skewness | 1.5369 | (P < 0.0001) |
| Coefficient of Kurtosis | 1.8249 | (P = 0.0040) |

| | |
|---|---|
| D'Agostino-Pearson test | reject Normality (P < 0.0001) |
| for Normal distribution | |

| Percentiles | | 95% Confidence interval |
|---|---|---|
| 2.5 | 0.0000 | |
| 5 | 0.0000 | 0.0000 to 0.0000 |
| 10 | 0.0000 | 0.0000 to 0.0000 |
| 25 | 1.0000 | 0.0000 to 1.0000 |
| 75 | 6.2500 | 5.0000 to 8.9203 |
| 90 | 12.6000 | 9.5756 to 18.0000 |
| 95 | 18.0000 | 13.0000 to 20.0000 |
| 97.5 | 20.0000 | |

TABLE 6B
Summary statistics

| | |
|---|---|
| Variable | ADHD_95th_percentile |

| Sample size | 137 | |
|---|---|---|
| Lowest value | 0.0000 | |
| Highest value | 20.0000 | |
| Arithmetic mean | 2.9124 | |
| 95% CI for the mean | 2.1586 to 3.6662 | |
| Median | 1.0000 | |
| 95% CI for the median | 1.0000 to 2.0000 | |
| Variance | 19.9040 | |
| Standard deviation | 4.4614 | |
| Relative standard deviation | 1.5319 | (153.19%) |
| Standard error of the mean | 0.3812 | |
| Coefficient of Skewness | 2.4667 | (P < 0.0001) |
| Coefficient of Kurtosis | 6.0423 | (P < 0.0001) |

| | |
|---|---|
| D'Agostino-Pearson test | reject Normality (P < 0.0001) |
| for Normal distribution | |

| Percentiles | | 95% Confidence interval |
|---|---|---|
| 2.5 | 0.0000 | |
| 5 | 0.0000 | 0.0000 to 0.0000 |
| 10 | 0.0000 | 0.0000 to 0.0000 |
| 25 | 0.0000 | 0.0000 to 0.0000 |
| 75 | 4.0000 | 3.0000 to 5.0000 |
| 90 | 7.0000 | 5.0000 to 16.0000 |
| 95 | 16.0000 | 7.0000 to 20.0000 |
| 97.5 | 19.0750 | |

TABLE 7A
Summary statistics

| | |
|---|---|
| Variable | Non_ADHD_90th_percentile |
| | Non ADHD 90th percentile |

| Sample size | 132 | |
|---|---|---|
| Lowest value | 0.0000 | |
| Highest value | 19.0000 | |
| Arithmetic mean | 1.9621 | |
| 95% CI for the mean | 1.4352 to 2.4890 | |
| Median | 1.0000 | |
| 95% CI for the median | 1.0000 to 1.0000 | |
| Variance | 9.3650 | |
| Standard deviation | 3.0602 | |
| Relative standard deviation | 1.5597 | (155.97%) |
| Standard error of the mean | 0.2664 | |
| Coefficient of Skewness | 2.7058 | (P < 0.0001) |
| Coefficient of Kurtosis | 8.9629 | (P < 0.0001) |

| | |
|---|---|
| D'Agostino-Pearson test | reject Normality (P < 0.0001) |
| for Normal distribution | |

| Percentiles | | 95% Confidence interval |
|---|---|---|
| 2.5 | 0.0000 | |
| 5 | 0.0000 | 0.0000 to 0.0000 |
| 10 | 0.0000 | 0.0000 to 0.0000 |
| 25 | 0.0000 | 0.0000 to 0.0000 |
| 75 | 2.0000 | 2.0000 to 3.0000 |
| 90 | 5.3000 | 4.0000 to 8.6297 |
| 95 | 8.9000 | 6.0000 to 12.0000 |
| 97.5 | 12.0000 | |

TABLE 7B
Summary statistics

| | |
|---|---|
| Variable | Non_ADHD_95th_percentile |

| Sample size | 132 | |
|---|---|---|
| Lowest value | 0.0000 | |
| Highest value | 10.0000 | |
| Arithmetic mean | 0.9848 | |
| 95% CI for the mean | 0.6707 to 1.2990 | |
| Median | 0.0000 | |
| 95% CI for the median | 0.0000 to 0.0000 | |
| Variance | 3.3280 | |
| Standard deviation | 1.8243 | |
| Relative standard deviation | 1.8524 | (185.24%) |
| Standard error of the mean | 0.1588 | |
| Coefficient of Skewness | 2.7344 | (P < 0.0001) |
| Coefficient of Kurtosis | 8.2646 | (P < 0.0001) |

| | |
|---|---|
| D'Agostino-Pearson test | reject Normality (P < 0.0001) |
| for Normal distribution | |

| Percentiles | | 95% Confidence interval |
|---|---|---|
| 2.5 | 0.0000 | |
| 5 | 0.0000 | 0.0000 to 0.0000 |
| 10 | 0.0000 | 0.0000 to 0.0000 |
| 25 | 0.0000 | 0.0000 to 0.0000 |
| 75 | 1.0000 | 1.0000 to 2.0000 |
| 90 | 3.0000 | 2.0000 to 5.2594 |
| 95 | 5.8000 | 3.0000 to 7.6200 |
| 97.5 | 7.0000 | |

TABLE 8A
Summary statistics

| | |
|---|---|
| Variable | ADHD_90th_percentile_1 |
| | ADHD 90th percentile_1 |

Back-transformed after logarithmic transformation.

| Sample size | 132 |
|---|---|
| Lowest value | 0.1000 |
| Highest value | 19.0000 |
| Geometric mean | 0.6626 |
| 95% CI for the mean | 0.4997 to 0.8786 |
| Median | 1.0000 |
| 95% CI for the median | 1.0000 to 1.0000 |
| Coefficient of Skewness | 0.03362 (P = 0.8699) |
| Coefficient of Kurtosis | −1.3824 (P < 0.0001) |
| D'Agostino-Pearson test | reject Normality (P < 0.0001) |
| for Normal distribution | |

| Percentiles | | 95% Confidence interval |
|---|---|---|
| 2.5 | 0.10000 | |
| 5 | 0.10000 | 0.10000 to 0.10000 |
| 10 | 0.10000 | 0.10000 to 0.10000 |
| 25 | 0.10000 | 0.10000 to 0.10000 |
| 75 | 2.0000 | 2.0000 to 3.0000 |
| 90 | 5.2811 | 4.0000 to 8.6159 |
| 95 | 8.8946 | 6.0000 to 12.0000 |
| 97.5 | 12.0000 | |

TABLE 8B
Summary statistics

| | |
|---|---|
| Variable | ADHD_95th_percentile_1 |

Back-transformed after logarithmic transformation.

| Sample size | 137 |
|---|---|
| Lowest value | 0.1000 |
| Highest value | 20.0000 |
| Geometric mean | 0.8838 |
| 95% CI for the mean | 0.6542 to 1.1940 |
| Median | 1.0000 |
| 95% CI for the median | 1.0000 to 2.0000 |
| Coefficient of Skewness | −0.09449 (P = 0.6402) |
| Coefficient of Kurtosis | −1.4081 (P < 0.0001) |
| D'Agostino-Pearson test | reject Normality (P < 0.0001) |
| for Normal distribution | |

| Percentiles | | 95% Confidence interval |
|---|---|---|
| 2.5 | 0.10000 | |
| 5 | 0.10000 | 0.10000 to 0.10000 |
| 10 | 0.10000 | 0.10000 to 0.10000 |
| 25 | 0.10000 | 0.10000 to 0.10000 |
| 75 | 4.0000 | 3.0000 to 5.0000 |
| 90 | 7.0000 | 5.0000 to 16.0000 |
| 95 | 16.0000 | 7.0000 to 20.0000 |
| 97.5 | 19.0732 | |

TABLE 9A
Summary statistics

| | |
|---|---|
| Variable | Non_ADHD_90th_percentile_1 |

Back-transformed after logarithmic transformation.

| Sample size | 132 |
|---|---|
| Lowest value | 0.1000 |
| Highest value | 19.0000 |
| Geometric mean | 0.6626 |
| 95% CI for the mean | 0.4997 to 0.8786 |
| Median | 1.0000 |
| 95% CI for the median | 1.0000 to 1.0000 |
| Coefficient of Skewness | 0.03362 (P = 0.8699) |
| Coefficient of Kurtosis | −1.3824 (P < 0.0001) |
| D'Agostino-Pearson test | reject Normality (P < 0.0001) |
| for Normal distribution | |

| Percentiles | | 95% Confidence interval |
|---|---|---|
| 2.5 | 0.10000 | |
| 5 | 0.10000 | 0.10000 to 0.10000 |
| 10 | 0.10000 | 0.10000 to 0.10000 |
| 25 | 0.10000 | 0.10000 to 0.10000 |
| 75 | 2.0000 | 2.0000 to 3.0000 |
| 90 | 5.2811 | 4.0000 to 8.6159 |
| 95 | 8.8946 | 6.0000 to 12.0000 |
| 97.5 | 12.0000 | |

TABLE 9B
Summary statistics

| | |
|---|---|
| Variable | Non_ADHD_95th_percentile_1 |

Back-transformed after logarithmic transformation.

| Sample size | 132 |
|---|---|
| Lowest value | 0.1000 |
| Highest value | 10.0000 |
| Geometric mean | 0.3258 |
| 95% CI for the mean | 0.2518 to 0.4216 |
| Median | 0.10000 |
| 95% CI for the median | 0.10000 to 0.10000 |
| Coefficient of Skewness | 0.6874 (P = 0.0020) |
| Coefficient of Kurtosis | −1.1003 (P < 0.0001) |
| D'Agostino-Pearson test | reject Normality (P < 0.0001) |
| for Normal distribution | |

| Percentiles | | 95% Confidence interval |
|---|---|---|
| 2.5 | 0.10000 | |
| 5 | 0.10000 | 0.10000 to 0.10000 |
| 10 | 0.10000 | 0.10000 to 0.10000 |
| 25 | 0.10000 | 0.10000 to 0.10000 |
| 75 | 1.0000 | 1.0000 to 2.0000 |
| 90 | 3.0000 | 2.0000 to 5.1635 |
| 95 | 5.7616 | 3.0000 to 7.5672 |
| 97.5 | 7.0000 | |

TABLE 10A
Independent samples t-test
Sample 1

| | |
|---|---|
| Variable | ADHD_90th_percentile_1 |

Sample 2

| | |
|---|---|
| Variable | Non_ADHD_90th_percentile_1 |

Back-transformed after logarithmic transformation.

| | Sample 1 | Sample 2 |
|---|---|---|
| Sample size | 137 | 132 |
| Geometric mean | 1.6449 | 0.6626 |
| 95% CI for the mean | 1.2191 to 2.2194 | 0.4997 to 0.8786 |
| Variance of Logs | 0.5930 | 0.5065 |

| | |
|---|---|
| F-test for equal variances | P = 0.364 |

T-test (assuming equal variances)
Difference on Log-transformed scale

| | |
|---|---|
| Difference | −0.3949 |
| Standard Error | 0.09049 |
| 95% CI of difference | −0.5730 to −0.2167 |
| Test statistic t | −4.363 |
| Degrees of Freedom (DF) | 267 |
| Two-tailed probability | P < 0.0001 |

Back-transformed results

| | |
|---|---|
| Ratio of geometric means | 0.4028 |
| 95% CI of ratio | 0.2673 to 0.6072 |

TABLE 10B
Independent samples t-test
Sample 1

| | |
|---|---|
| Variable | ADHD_95th_percentile_1 |

Sample 2

| | |
|---|---|
| Variable | Non_ADHD_95th_percentile_1 |

Back-transformed after logarithmic transformation.

| | Sample 1 | Sample 2 |
|---|---|---|
| Sample size | 137 | 132 |
| Geometric mean | 0.8838 | 0.3258 |
| 95% CI for the mean | 0.6542 to 1.1940 | 0.2518 to 0.4216 |
| Variance of Logs | 0.5978 | 0.4228 |

| | |
|---|---|
| F-test for equal variances | P = 0.047 |

T-test (assuming equal variances)
Difference on Log-transformed scale

| | |
|---|---|
| Difference | −0.4334 |
| Standard Error | 0.08726 |
| 95% CI of difference | −0.6052 to −0.2616 |
| Test statistic t | −4.967 |
| Degrees of Freedom (DF) | 267 |
| Two-tailed probability | P < 0.0001 |

Back-transformed results

| | |
|---|---|
| Ratio of geometric means | 0.3686 |
| 95% CI of ratio | 0.2482 to 0.5475 |

TABLE 11A
Mann-Whitney test (independent samples)
Sample 1

| | |
|---|---|
| Variable | ADHD_90th_percentile |
| | ADHD 90th percentile |

Sample 2

| | |
|---|---|
| Variable | Non_ADHD_90th_percentile |
| | Non_ADHD 90th percentile |

| | Sample 1 | Sample 2 |
|---|---|---|
| Sample size | 137 | 132 |
| Lowest value | 0.0000 | 0.0000 |
| Highest value | 20.0000 | 19.0000 |
| Median | 3.0000 | 1.0000 |
| 95% CI for the median | 2.0000 to 3.0000 | 1.0000 to 1.0000 |
| Interquartile range | 1.0000 to 6.2500 | 0.0000 to 2.0000 |

Mann-Whitney test (independent samples)

| | |
|---|---|
| Average rank of first group | 156.1679 |
| Average rank of second group | 113.0303 |
| Mann-Whitney U | 6142.00 |
| Test statistic Z (corrected for ties) | 4.637 |
| Two-tailed probability | P < 0.0001 |

TABLE 11B
Mann-Whitney test (independent samples)
Sample 1

| | |
|---|---|
| Variable | ADHD_95th_percentile |

Sample 2

| | |
|---|---|
| Variable | Non_ADHD_95th_percentile |

| | Sample 1 | Sample 2 |
|---|---|---|
| Sample size | 137 | 132 |
| Lowest value | 0.0000 | 0.0000 |
| Highest value | 20.0000 | 10.0000 |
| Median | 1.0000 | 0.0000 |
| 95% CI for the median | 1.0000 to 2.0000 | 0.0000 to 0.0000 |
| Interquartile range | 0.0000 to 4.0000 | 0.0000 to 1.0000 |

Mann-Whitney test (independent samples)

| | |
|---|---|
| Average rank of first group | 156.0401 |
| Average rank of second group | 113.1629 |
| Mann-Whitney U | 6159.50 |
| Test statistic Z (corrected for ties) | 4.792 |
| Two-tailed probability | P < 0.0001 |

TABLE 12A
ROC curve

| | |
|---|---|
| Variable | ADHD_Test_90th |
| | ADHD Test 90th |
| Classification variable | Diagnosis_1_ADHD_0_Non_ADHD_ |
| | Diagnosis(1_ADHD 0_Non_ADHD) |

| Sample size | 269 |
|---|---|
| Positive group ^{a} | 137 (50.93%) |
| Negative group ^{b} | 132 (49.07%) |

^{a} Diagnosis_1_ADHD_0_Non_ADHD_ = 1
^{b} Diagnosis_1_ADHD_0_Non_ADHD_ = 0

| | |
|---|---|
| Disease prevalence (%) | unknown |

Area under the ROC curve (AUC)

| | |
|---|---|
| Area under the ROC curve (AUC) | 0.660 |
| Standard Error ^{a} | 0.0326 |
| 95% Confidence interval ^{b} | 0.600 to 0.717 |
| z statistic | 4.925 |
| Significance level P (Area = 0.5) | <0.0001 |

^{a} DeLong et al., 1988
^{b} Binomial exact
Youden index

| | |
|---|---|
| Youden index J | 0.2980 |
| 95% Confidence interval ^{a} | 0.1810 to 0.4018 |
| Associated criterion | >2 |
| 95% Confidence interval ^{a} | >1 to >5 |
| Sensitivity | 53.28 |
| Specificity | 76.52 |

^{a} BCₐ bootstrap confidence interval (1000 iterations; random number seed: 978).

TABLE 12B
ROC curve

| | |
|---|---|
| Variable | ADHD_Test_95th |
| | ADHD Test 95th |
| Classification variable | Diagnosis_1_ADHD_0_Non_ADHD_ |
| | Diagnosis(1_ADHD 0_Non_ADHD) |

| Sample size | 269 |
|---|---|
| Positive group ^{a} | 137 (50.93%) |
| Negative group ^{b} | 132 (49.07%) |

| ^{a} Diagnosis_1_ADHD_0_Non_ADHD_ = 1 | |
|---|---|
| ^{b} Diagnosis_1_ADHD_0_Non_ADHD_ = 0 | |
| Disease prevalence (%) | unknown |

Area under the ROC curve (AUC)

| | |
|---|---|
| Area under the ROC curve (AUC) | 0.659 |
| Standard Error ^{a} | 0.0312 |
| 95% Confidence interval ^{b} | 0.599 to 0.716 |
| z statistic | 5.117 |
| Significance level P (Area = 0.5) | <0.0001 |

^{a} DeLong et al., 1988
^{b} Binomial exact

Youden index

| | |
|---|---|
| Youden index J | 0.2851 |
| 95% Confidence interval ^{a} | 0.1724 to 0.3749 |
| Associated criterion | >1 |
| 95% Confidence interval ^{a} | >0 to >2 |
| Sensitivity | 47.45 |
| Specificity | 81.06 |

^{a} BCₐ bootstrap confidence interval (1000 iterations; random number seed: 978).

TABLE 13A
Performance Metrics in Predicting ADD/ADHD Status from Number of Positive
Foods Using 90th Percentile of ELISA Signal to determine Positive

| | No. of | | | Positive | Negative | Overall |
|---|---|---|---|---|---|---|
| | Positive Foods | | | Predictive | Predictive | Percent |
| Sex | as Cutoff | Sensitivity | Specificity | Value | Value | Agreement |
| FEMALE | 1 | 0.85 | 0.26 | 0.67 | 0.48 | 0.64 |
| | 2 | 0.75 | 0.40 | 0.70 | 0.48 | 0.63 |
| | 3 | 0.67 | 0.55 | 0.73 | 0.48 | 0.63 |
| | 4 | 0.60 | 0.66 | 0.76 | 0.48 | 0.63 |
| | 5 | 0.56 | 0.72 | 0.78 | 0.47 | 0.62 |
| | 6 | 0.52 | 0.76 | 0.79 | 0.46 | 0.60 |
| | 7 | 0.48 | 0.77 | 0.79 | 0.45 | 0.59 |
| | 8 | 0.45 | 0.80 | 0.80 | 0.44 | 0.57 |
| | 9 | 0.42 | 0.82 | 0.81 | 0.44 | 0.56 |
| | 10 | 0.38 | 0.85 | 0.82 | 0.43 | 0.55 |
| | 11 | 0.35 | 0.88 | 0.83 | 0.43 | 0.53 |
| | 12 | 0.33 | 0.89 | 0.83 | 0.42 | 0.53 |
| | 13 | 0.30 | 0.90 | 0.84 | 0.42 | 0.52 |
| | 14 | 0.28 | 0.91 | 0.84 | 0.41 | 0.50 |
| | 15 | 0.25 | 0.91 | 0.83 | 0.40 | 0.49 |
| | 16 | 0.23 | 0.92 | 0.83 | 0.40 | 0.48 |
| | 17 | 0.21 | 0.93 | 0.83 | 0.39 | 0.47 |
| | 18 | 0.20 | 0.94 | 0.85 | 0.39 | 0.46 |
| | 19 | 0.18 | 0.94 | 0.86 | 0.39 | 0.45 |
| | 20 | 0.16 | 0.96 | 0.88 | 0.39 | 0.45 |
| | 21 | 0.15 | 0.97 | 0.89 | 0.39 | 0.44 |
| | 22 | 0.13 | 0.97 | 0.90 | 0.38 | 0.43 |
| | 23 | 0.12 | 0.98 | 0.93 | 0.38 | 0.43 |
| | 24 | 0.10 | 1.00 | 1.00 | 0.38 | 0.42 |
| | 25 | 0.09 | 1.00 | 1.00 | 0.38 | 0.41 |
| | 26 | 0.08 | 1.00 | 1.00 | 0.38 | 0.41 |
| | 27 | 0.07 | 1.00 | 1.00 | 0.38 | 0.40 |
| | 28 | 0.07 | 1.00 | 1.00 | 0.37 | 0.40 |
| | 29 | 0.07 | 1.00 | 1.00 | 0.37 | 0.40 |
| | 30 | 0.07 | 1.00 | 1.00 | 0.37 | 0.40 |
| | 31 | 0.06 | 1.00 | 1.00 | 0.37 | 0.40 |
| | 32 | 0.05 | 1.00 | 1.00 | 0.37 | 0.39 |
| | 33 | 0.05 | 1.00 | 1.00 | 0.37 | 0.39 |
| | 34 | 0.04 | 1.00 | 1.00 | 0.37 | 0.38 |
| | 35 | 0.04 | 1.00 | 1.00 | 0.36 | 0.38 |
| | 36 | 0.02 | 1.00 | 1.00 | 0.36 | 0.37 |
| | 37 | 0.02 | 1.00 | 1.00 | 0.36 | 0.37 |

TABLE 13B
Performance Metrics in Predicting ADD/ADHD Status from Number of Positive
Foods Using 90th Percentile of ELISA Signal to determine Positive

| | No. of | | | Positive | Negative | Overall |
|---|---|---|---|---|---|---|
| | Positive Foods | | | Predictive | Predictive | Percent |
| Sex | as Cutoff | Sensitivity | Specificity | Value | Value | Agreement |
| MALE | 1 | 0.89 | 0.25 | 0.40 | 0.80 | 0.48 |
| | 2 | 0.77 | 0.44 | 0.43 | 0.78 | 0.56 |
| | 3 | 0.65 | 0.57 | 0.45 | 0.74 | 0.60 |
| | 4 | 0.57 | 0.66 | 0.48 | 0.73 | 0.63 |
| | 5 | 0.50 | 0.73 | 0.52 | 0.73 | 0.65 |
| | 6 | 0.44 | 0.79 | 0.54 | 0.72 | 0.67 |
| | 7 | 0.37 | 0.83 | 0.55 | 0.70 | 0.67 |
| | 8 | 0.33 | 0.85 | 0.56 | 0.70 | 0.66 |
| | 9 | 0.29 | 0.87 | 0.55 | 0.69 | 0.66 |
| | 10 | 0.27 | 0.88 | 0.56 | 0.69 | 0.66 |
| | 11 | 0.26 | 0.90 | 0.58 | 0.68 | 0.67 |
| | 12 | 0.24 | 0.91 | 0.60 | 0.68 | 0.67 |
| | 13 | 0.23 | 0.92 | 0.60 | 0.68 | 0.67 |
| | 14 | 0.21 | 0.92 | 0.58 | 0.68 | 0.67 |
| | 15 | 0.18 | 0.92 | 0.57 | 0.67 | 0.66 |
| | 16 | 0.15 | 0.93 | 0.56 | 0.67 | 0.65 |
| | 17 | 0.12 | 0.94 | 0.50 | 0.66 | 0.65 |
| | 18 | 0.11 | 0.94 | 0.50 | 0.66 | 0.64 |
| | 19 | 0.10 | 0.94 | 0.50 | 0.65 | 0.64 |
| | 20 | 0.09 | 0.95 | 0.50 | 0.65 | 0.64 |
| | 21 | 0.08 | 0.96 | 0.50 | 0.65 | 0.64 |
| | 22 | 0.08 | 0.96 | 0.50 | 0.65 | 0.64 |
| | 23 | 0.07 | 0.96 | 0.50 | 0.65 | 0.65 |
| | 24 | 0.07 | 0.97 | 0.60 | 0.65 | 0.65 |
| | 25 | 0.07 | 0.98 | 0.67 | 0.65 | 0.65 |
| | 26 | 0.07 | 0.98 | 0.67 | 0.65 | 0.65 |
| | 27 | 0.07 | 0.98 | 0.67 | 0.65 | 0.65 |
| | 28 | 0.07 | 0.98 | 0.67 | 0.65 | 0.65 |
| | 29 | 0.04 | 0.98 | 0.67 | 0.65 | 0.65 |
| | 30 | 0.04 | 0.98 | 0.67 | 0.65 | 0.65 |
| | 31 | 0.04 | 0.98 | 0.67 | 0.65 | 0.65 |
| | 32 | 0.04 | 1.00 | 0.67 | 0.65 | 0.65 |
| | 33 | 0.03 | 1.00 | 1.00 | 0.65 | 0.65 |
| | 34 | 0.00 | 1.00 | 1.00 | 0.65 | 0.65 |
| | 35 | 0.00 | 1.00 | 1.00 | 0.64 | 0.64 |
| | 36 | 0.00 | 1.00 | 0.00 | 0.64 | 0.64 |
| | 37 | 0.00 | 1.00 | 0.00 | 0.64 | 0.64 |

TABLE 14A
Performance Metrics in Predicting ADD/ADHD Status from Number of Positive
Foods Using 95th Percentile of ELISA Signal to determine Positive

| | No. of | | | Positive | Negative | Overall |
|---|---|---|---|---|---|---|
| | Positive Foods | | | Predictive | Predictive | Percent |
| Sex | as Cutoff | Sensitivity | Specificity | Value | Value | Agreement |
| FEMALE | 1 | 0.73 | 0.41 | 0.69 | 0.46 | 0.62 |
| | 2 | 0.61 | 0.58 | 0.72 | 0.45 | 0.60 |
| | 3 | 0.53 | 0.72 | 0.77 | 0.46 | 0.60 |
| | 4 | 0.47 | 0.79 | 0.81 | 0.46 | 0.59 |
| | 5 | 0.43 | 0.82 | 0.81 | 0.45 | 0.57 |
| | 6 | 0.39 | 0.84 | 0.81 | 0.43 | 0.55 |
| | 7 | 0.35 | 0.85 | 0.81 | 0.42 | 0.53 |
| | 8 | 0.31 | 0.88 | 0.82 | 0.41 | 0.51 |
| | 9 | 0.27 | 0.91 | 0.84 | 0.41 | 0.49 |
| | 10 | 0.23 | 0.93 | 0.85 | 0.40 | 0.48 |
| | 11 | 0.20 | 0.94 | 0.86 | 0.39 | 0.47 |
| | 12 | 0.18 | 0.94 | 0.86 | 0.39 | 0.45 |
| | 13 | 0.16 | 0.97 | 0.88 | 0.39 | 0.44 |
| | 14 | 0.14 | 0.97 | 0.89 | 0.38 | 0.44 |
| | 15 | 0.12 | 0.97 | 0.91 | 0.38 | 0.43 |
| | 16 | 0.11 | 1.00 | 1.00 | 0.38 | 0.42 |
| | 17 | 0.10 | 1.00 | 1.00 | 0.38 | 0.42 |
| | 18 | 0.09 | 1.00 | 1.00 | 0.38 | 0.41 |
| | 19 | 0.08 | 1.00 | 1.00 | 0.38 | 0.41 |
| | 20 | 0.07 | 1.00 | 1.00 | 0.37 | 0.40 |
| | 21 | 0.07 | 1.00 | 1.00 | 0.37 | 0.40 |
| | 22 | 0.07 | 1.00 | 1.00 | 0.37 | 0.40 |
| | 23 | 0.07 | 1.00 | 1.00 | 0.37 | 0.40 |
| | 24 | 0.07 | 1.00 | 1.00 | 0.37 | 0.40 |
| | 25 | 0.06 | 1.00 | 1.00 | 0.37 | 0.40 |
| | 26 | 0.06 | 1.00 | 1.00 | 0.37 | 0.39 |
| | 27 | 0.05 | 1.00 | 1.00 | 0.37 | 0.39 |
| | 28 | 0.05 | 1.00 | 1.00 | 0.37 | 0.39 |
| | 29 | 0.05 | 1.00 | 1.00 | 0.37 | 0.38 |
| | 30 | 0.04 | 1.00 | 1.00 | 0.37 | 0.38 |
| | 31 | 0.04 | 1.00 | 1.00 | 0.36 | 0.38 |
| | 32 | 0.03 | 1.00 | 1.00 | 0.36 | 0.38 |
| | 33 | 0.03 | 1.00 | 1.00 | 0.36 | 0.38 |
| | 34 | 0.02 | 1.00 | 1.00 | 0.36 | 0.37 |
| | 35 | 0.02 | 1.00 | 1.00 | 0.36 | 0.37 |
| | 36 | 0.02 | 1.00 | 1.00 | 0.36 | 0.36 |
| | 37 | 0.02 | 1.00 | 1.00 | 0.36 | 0.36 |

TABLE 14B
Performance Metrics in Predicting ADD/ADHD Status from Number of Positive
Foods Using 95th Percentile of ELISA Signal to determine Positive

| | No. of | | | Positive | Negative | Overall |
|---|---|---|---|---|---|---|
| | Positive Foods | | | Predictive | Predictive | Percent |
| Sex | as Cutoff | Sensitivity | Specificity | Value | Value | Agreement |
| MALE | 1 | 0.80 | 0.43 | 0.44 | 0.80 | 0.57 |
| | 2 | 0.65 | 0.63 | 0.49 | 0.76 | 0.64 |
| | 3 | 0.50 | 0.73 | 0.50 | 0.72 | 0.65 |
| | 4 | 0.41 | 0.82 | 0.56 | 0.71 | 0.67 |
| | 5 | 0.33 | 0.87 | 0.59 | 0.70 | 0.68 |
| | 6 | 0.28 | 0.90 | 0.60 | 0.69 | 0.68 |
| | 7 | 0.24 | 0.91 | 0.60 | 0.69 | 0.67 |
| | 8 | 0.21 | 0.92 | 0.62 | 0.68 | 0.67 |
| | 9 | 0.18 | 0.94 | 0.60 | 0.67 | 0.67 |
| | 10 | 0.15 | 0.94 | 0.57 | 0.67 | 0.66 |
| | 11 | 0.12 | 0.94 | 0.57 | 0.66 | 0.65 |
| | 12 | 0.10 | 0.95 | 0.50 | 0.66 | 0.65 |
| | 13 | 0.08 | 0.96 | 0.50 | 0.65 | 0.64 |
| | 14 | 0.07 | 0.96 | 0.50 | 0.65 | 0.64 |
| | 15 | 0.07 | 0.96 | 0.50 | 0.65 | 0.65 |
| | 16 | 0.07 | 0.97 | 0.50 | 0.65 | 0.65 |
| | 17 | 0.07 | 0.98 | 0.60 | 0.65 | 0.65 |
| | 18 | 0.07 | 0.98 | 0.67 | 0.65 | 0.65 |
| | 19 | 0.07 | 0.98 | 0.67 | 0.65 | 0.65 |
| | 20 | 0.07 | 0.98 | 0.67 | 0.65 | 0.65 |
| | 21 | 0.07 | 0.98 | 0.67 | 0.65 | 0.66 |
| | 22 | 0.07 | 0.98 | 0.75 | 0.65 | 0.66 |
| | 23 | 0.07 | 1.00 | 1.00 | 0.65 | 0.66 |
| | 24 | 0.07 | 1.00 | 1.00 | 0.66 | 0.66 |
| | 25 | 0.07 | 1.00 | 1.00 | 0.66 | 0.66 |
| | 26 | 0.06 | 1.00 | 1.00 | 0.65 | 0.66 |
| | 27 | 0.04 | 1.00 | 1.00 | 0.65 | 0.66 |
| | 28 | 0.04 | 1.00 | 1.00 | 0.65 | 0.66 |
| | 29 | 0.04 | 1.00 | 1.00 | 0.65 | 0.66 |
| | 30 | 0.04 | 1.00 | 1.00 | 0.65 | 0.66 |
| | 31 | 0.03 | 1.00 | 1.00 | 0.65 | 0.65 |
| | 32 | 0.00 | 1.00 | 1.00 | 0.65 | 0.65 |
| | 33 | 0.00 | 1.00 | 1.00 | 0.64 | 0.64 |
| | 34 | 0.00 | 1.00 | 1.00 | 0.64 | 0.64 |
| | 35 | 0.00 | 1.00 | 1.00 | 0.64 | 0.64 |
| | 36 | 0.00 | 1.00 | . | 0.64 | 0.64 |
| | 37 | 0.00 | 1.00 | . | 0.64 | 0.64 |

1. An Attention Deficit Disorder/Attention Deficit Hyperactivity Disorder (ADD/ADHD) test panel consisting essentially of:
a plurality of distinct ADD/ADHD trigger food preparations immobilized to an individually addressable solid carrier;
wherein the plurality of distinct ADD/ADHD trigger food preparations each have an ADD/ADHD raw p-value of ≤0.07 or an ADD/ADHD false discovery rate (FDR) multiplicity adjusted p-value of ≤0.10.

2.-4. (canceled)

5. The test panel of claim 1, wherein the plurality of distinct ADD/ADHD trigger food preparations includes at least eight food preparations.

6. The test panel of claim 1, wherein the plurality of distinct ADD/ADHD trigger food preparations includes at least 12 food preparations.

7. (canceled)

8. The test panel of claim 1 wherein the plurality of distinct ADD/ADHD trigger food preparations each have an ADD/ADHD raw p-value of ≤0.05 or an ADD/ADHD false discovery rate (FDR) multiplicity adjusted p-value of ≤0.08.

9. The test panel of claim 1 wherein the plurality of distinct ADD/ADHD trigger food preparations each have an ADD/ADHD raw p-value of ≤0.025 or an ADD/ADHD false discovery rate (FDR) multiplicity adjusted p-value of ≤0.07.

10. The test panel of claim 1 wherein the ADD/ADHD FDR multiplicity adjusted p-value is adjusted for at least one of age or gender.

11.-14. (canceled)

15. The test panel of claim 1 wherein the plurality of distinct ADD/ADHD trigger food preparations is a crude filtered aqueous extract or a processed aqueous extract.

16. (canceled)

17. The test panel of claim 1 wherein the solid carrier is a well of a multiwell plate, a bead, an electrical, a chemical sensor, a microchip or an adsorptive film.

18. A method of testing food sensitivity comprising:
contacting a test panel consisting essentially of a plurality of distinct Attention Deficit Disorder/Attention Deficit Hyperactivity Disorder (ADD/ADHD) trigger food preparations with a bodily fluid of a patient that is diagnosed with or suspected of having ADD/ADHD,
wherein the step of contacting is performed under conditions that allow at least a portion of an immunoglobulin from the bodily fluid to bind to at least one component of the plurality of distinct ADD/ADHD trigger food preparations;
measuring the immunoglobulin bound to the at least one component of the plurality of distinct ADD/ADHD trigger food preparations to obtain a signal; and
updating or generating a report using the signal.

19.-22. (canceled)

23. The method of claim 18 wherein the plurality of distinct ADD/ADHD trigger food preparations each have an ADD/ADHD raw p-value of ≤0.07 or an ADD/ADHD false discovery rate (FDR) multiplicity adjusted p-value of ≤0.10.

24. The method of claim 18 wherein the plurality of distinct ADD/ADHD trigger food preparations each have an ADD/ADHD raw p-value of ≤0.05 or an ADD/ADHD false discovery rate (FDR) multiplicity adjusted p-value of ≤0.08.

25.-29. (canceled)

30. The method of claim 18, further comprising comparing the signal to a gender-stratified reference value for the food preparation using gender identification to obtain a result, wherein the gender-stratified reference value for the food preparation is at least a 90^{th} percentile value.

31. A method of generating a test for food sensitivity in a patient diagnosed with or suspected of having Attention Deficit Disorder/Attention Deficit Hyperactivity Disorder (ADD/ADHD), comprising:
obtaining test results for a plurality of distinct food preparations, wherein the test results are based on bodily fluids of patients diagnosed with or suspected of having ADD/ADHD and bodily fluids of a control group not diagnosed with or not suspected of having ADD/ADHD;
stratifying the test results by gender for each of the distinct food preparations;
assigning for a predetermined percentile rank a different cutoff value for male and female patients for each of the distinct food preparations;
selecting a plurality of distinct ADD/ADHD trigger food preparations that each have an ADD/ADHD raw p-value of ≤0.07 or an ADD/ADHD FDR multiplicity adjusted p-value of ≤0.10; and
generating a test consisting essentially of the selected distinct ADD/ADHD trigger food preparations.

32. (canceled)

33. The method of claim 31 wherein the plurality of distinct food preparations includes at least eight distinct ADD/ADHD trigger food preparations.

34. The method of claim 31 wherein the plurality of distinct food preparations includes at least ten distinct ADD/ADHD trigger food preparations.

35.-38. (canceled)

39. The method of claim 31 wherein the predetermined percentile rank is an at least 90^{th} percentile rank.

40.-70. (canceled)

71. A detection apparatus comprising:
a plurality of distinct ADD/ADHD trigger food preparations immobilized to an individually addressable solid carrier;
wherein the plurality of distinct ADD/ADHD trigger food preparations having an average ADD/ADHD raw p-value of ≤0.07 or an average ADD/ADHD false discovery rate (FDR) multiplicity adjusted p-value of ≤0.10.

72. (canceled)

73. The detection apparatus of claim 71, wherein the plurality of food preparations includes at least eight distinct ADD/ADHD trigger food preparations.

74.-126. (canceled)

127. The test panel of claim 1, wherein the plurality of distinct ADD/ADHD trigger food preparations includes at least two food preparations selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

128.-131. (canceled)

132. The detection apparatus of claim 71, wherein the plurality of food preparations includes at least two food preparations selected from the group consisting of cantaloupe, wheat, tomato, cucumber, squashes, almond, egg, cauliflower, pinto bean, broccoli, orange, butter, corn, lettuce, rye, peach, green pea, carrot, tea, mustard, strawberry, celery, green pepper, garlic, oat, onion, banana, eggplant, cabbage, safflower, olive, cottage cheese, grapefruit, walnut blk, cow milk, soybean, and chili pepper.

133. (canceled)
